SEQUENCE LISTING (240 sequences)

SEQ ID NO: 1 - DNA, 1571 bp, Lactobacillus plantarum
attaatttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctttct

SEQ ID NO: 2 - DNA, 46 bp, Lactobacillus plantarum
aactctggtattgattggtgcttgcatcatgatttacatttgagtg

SEQ ID NO: 3 - DNA, 71 bp, Lactobacillus plantarum
aacaacttggaccgcatggtccgagcttgaaagatggcttcggctatcacttttggatggtcccgcggcgt

SEQ ID NO: 4 - DNA, 63 bp, Lactobacillus plantarum
ggtttcggctcgtaaaactctgttgttaaagaagaacatatctgagagtaactgttcaggtat

SEQ ID NO: 5 - DNA, 85 bp, Lactobacillus plantarum
ttttttaagtctgatgtgaaagccttcggctcaaccgaagaagtgcatcggaaactgggaaacttgagtgcagaagaggacagtg

SEQ ID NO: 6 - DNA, 43 bp, Lactobacillus plantarum
aatgctaagtgttggagggtttccgcccttcagtgctgcagct

SEQ ID NO: 7 - DNA, 51 bp, Lactobacillus plantarum
catactatgcaaatctaagagattagacgttcccttcggggacatggatac

SEQ ID NO: 8 - DNA, 59 bp, Lactobacillus plantarum
ttattatcagttgccagcattaagttgggcactctggtgagactgccggtgacaaaccg

SEQ ID NO: 9 - DNA, 57 bp, Lactobacillus plantarum
gatggtacaacgagttgcgaactcgcgagagtaagctaatctcttaaagccattctc

SEQ ID NO: 10 - DNA, 46 bp, Lactobacillus plantarum
caccatgagagtttgtaacacccaaagtcggtggggtaacctttta

SEQ ID NO: 11 - DNA, 70 bp, Lactobacillus plantarum
attaatttgagagtttgatcctggctcaggacgaacgctggcggcgtgcctaatacatgcaagtcgaacg

SEQ ID NO: 12 - DNA, 81 bp, Lactobacillus plantarum
agtggcgaactggtgagtaacacgtgggaaacctgcccagaagcgggggataacacctggaaacagatgctaataccgcat

SEQ ID NO: 13 - DNA, 174 bp, Lactobacillus plantarum
attagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaag

SEQ ID NO: 14 - DNA, 109 bp, Lactobacillus plantarum
tgacggtatttaaccagaaagccacggctaactacgtgccagcagccgcggtaatacgtaggtggcaagcgttgtccggatttattgggcgtaaagcgagcgcaggcgg

SEQ ID NO: 15 - DNA, 148 bp, Lactobacillus plantarum
gaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatg

SEQ ID NO: 16 - DNA, 131 bp, Lactobacillus plantarum
aacgcattaagcattccgcctggggagtacggccgcaaggctgaaactcaaaggaattgacgggggcccgcacaagcggtggagcatgtggtttaattcgaagctacgcgaagaaccttaccaggtcttga

SEQ ID NO: 17 - DNA, 69 bp, Lactobacillus plantarum
aggtggtgcatggttgtcgtcagctcgtgtcgtgagatgttgggttaagtcccgcaacgagcgcaaccc

SEQ ID NO: 18 - DNA, 67 bp, Lactobacillus plantarum
gaggaaggtggggatgacgtcaaatcatcatgccccttatgacctgggctacacacgtgctacaatg

SEQ ID NO: 19 - DNA, 110 bp, Lactobacillus plantarum
agttcggattgtaggctgcaactcgcctacatgaagtcggaatcgctagtaatcgcggatcagcatgccgcggtgaatacgttcccgggccttgtacacaccgcccgtca

SEQ ID NO: 20 - DNA, 91 bp, Lactobacillus plantarum
ggaaccagccgcctaaggtgggacagatgattagggtgaagtcgtaacaaggtagccgtaggagaacctgcggctggatcacctcctttct

SEQ ID NO: 21 - DNA, 1566 bp, Lactobacillus rhamnosus
tatgagagtttgatcctggctcaggatgaacgctggcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctt

SEQ ID NO: 22 - DNA, 47 bp, Lactobacillus rhamnosus
agttctgattattgaaaggtgcttgcatcttgatttaattttgaacg

SEQ ID NO: 23 - DNA, 71 bp, Lactobacillus rhamnosus
aaatccaagaaccgcatggttcttggctgaaagatggcgtaagctatcgcttttggatggacccgcggcgt

SEQ ID NO: 24 - DNA, 63 bp, Lactobacillus rhamnosus
gctttcgggtcgtaaaactctgttgttggagaagaatggtcggcagagtaactgttgtcggcg

SEQ ID NO: 25 - DNA, 85 bp, Lactobacillus rhamnosus
ttttttaagtctgatgtgaaagccctcggcttaaccgaggaagtgcatcggaaactgggaaacttgagtgcagaagaggacagtg

SEQ ID NO: 26 - DNA, 43 bp, Lactobacillus rhamnosus
aatgctaggtgttggagggtttccgcccttcagtgccgcagct

SEQ ID NO: 27 - DNA, 51 bp, Lactobacillus rhamnosus
catcttttgatcacctgagagatcaggtttccccttcgggggcaaaatgac

SEQ ID NO: 28 - DNA, 59 bp, Lactobacillus rhamnosus
ttatgactagttgccagcatttagttgggcactctagtaagactgccggtgacaaaccg

SEQ ID NO: 29 - DNA, 57 bp, Lactobacillus rhamnosus
gatggtacaacgagttgcgagaccgcgaggtcaagctaatctcttaaagccattctc

SEQ ID NO: 30 - DNA, 48 bp, Lactobacillus rhamnosus
caccatgagagtttgtaacacccgaagccggtggcgtaacccttttag

SEQ ID NO: 31 - DNA, 65 bp, Lactobacillus rhamnosus
tatgagagtttgatcctggctcaggatgaacgctggcggcgtgcctaatacatgcaagtcgaacg

SEQ ID NO: 32 - DNA, 81 bp, Lactobacillus rhamnosus
agtggcggacgggtgagtaacacgtgggtaacctgcccttaagtgggggataacatttggaaacagatgctaataccgcat

SEQ ID NO: 33 - DNA, 174 bp, Lactobacillus rhamnosus
attagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaag

SEQ ID NO: 34 - DNA, 109 bp, Lactobacillus rhamnosus
tgacggtatccaaccagaaagccacggctaactacgtgccagcagccgcggtaatacgtaggtggcaagcgttatccggatttattgggcgtaaagcgagcgcaggcgg

SEQ ID NO: 35 - DNA, 148 bp, Lactobacillus rhamnosus
gaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatg

SEQ ID NO: 36 - DNA, 131 bp, Lactobacillus rhamnosus
aacgcattaagcattccgcctggggagtacgaccgcaaggttgaaactcaaaggaattgacgggggcccgcacaagcggtggagcatgtggtttaattcgaagcaacgcgaagaaccttaccaggtcttga

SEQ ID NO: 37 - DNA, 69 bp, Lactobacillus rhamnosus; identical to SEQ ID NO: 17

SEQ ID NO: 38 - DNA, 67 bp, Lactobacillus rhamnosus; identical to SEQ ID NO: 18

SEQ ID NO: 39 - DNA, 110 bp, Lactobacillus rhamnosus
agttcggactgtaggctgcaactcgcctacacgaagtcggaatcgctagtaatcgcggatcagcacgccgcggtgaatacgttcccgggccttgtacacaccgcccgtca

SEQ ID NO: 40 - DNA, 88 bp, Lactobacillus rhamnosus
ggagcgagccgtctaaggtgggacaaatgattagggtgaagtcgtaacaaggtagccgtaggagaacctgcggctggatcacctcctt

SEQ ID NO: 41 - DNA, 1559 bp, Lactobacillus acidophilus
cgagagtttgatcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggcagggcagatgactggggtgaagtcgtaacaaggtagccgtaggagaacctgcggctggatcacctccttt

SEQ ID NO: 42 - DNA, 44 bp, Lactobacillus acidophilus
agctgaaccaacagattcacttcggtgatgacgttgggaacgcg

SEQ ID NO: 43 - DNA, 71 bp, Lactobacillus acidophilus
aagaaagcagatcgcatgatcagcttataaaaggcggcgtaagctgtcgctatgggatggccccgcggtgc

SEQ ID NO: 44 - DNA, 63 bp, Lactobacillus acidophilus
gttttcggatcgtaaagctctgttgttggtgaagaaggatagaggtagtaactggcctttatt

SEQ ID NO: 45 - DNA, 85 bp, Lactobacillus acidophilus
aagaataagtctgatgtgaaagccctcggcttaaccgaggaactgcatcggaaactgtttttcttgagtgcagaagaggagagtg

SEQ ID NO: 46 - DNA, 43 bp, Lactobacillus acidophilus
agtgctaagtgttgggaggtttccgcctctcagtgctgcagct

SEQ ID NO: 47 - DNA, 51 bp, Lactobacillus acidophilus
catctagtgcaatccgtagagatacggagttcccttcggggacactaagac

SEQ ID NO: 48 - DNA, 59 bp, Lactobacillus acidophilus
ttgtcattagttgccagcattaagttgggcactctaatgagactgccggtgacaaaccg

SEQ ID NO: 49 - DNA, 57 bp, Lactobacillus acidophilus
gacagtacaacgaggagcaagcctgcgaaggcaagcgaatctcttaaagctgttctc

SEQ ID NO: 50 - DNA, 45 bp, Lactobacillus acidophilus
caccatgggagtctgcaatgcccaaagccggtggcctaaccttcg

SEQ ID NO: 51 - DNA, 63 bp, Lactobacillus acidophilus
cgagagtttgatcctggctcaggacgaacgctggcggcgtgcctaatacatgcaagtcgagcg

SEQ ID NO: 52 - DNA, 81 bp, Lactobacillus acidophilus
agcggcggatgggtgagtaacacgtggggaacctgccccatagtctgggataccacttggaaacaggtgctaataccggat

SEQ ID NO: 53 - DNA, 174 bp, Lactobacillus acidophilus
attagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaag

SEQ ID NO: 54 - DNA, 109 bp, Lactobacillus acidophilus
tgacggtaatcaaccagaaagtcacggctaactacgtgccagcagccgcggtaatacgtaggtggcaagcgttgtccggatttattgggcgtaaagcgagcgcaggcgg

SEQ ID NO: 55 - DNA, 148 bp, Lactobacillus acidophilus
gaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatg

SEQ ID NO: 56 - DNA, 131 bp, Lactobacillus acidophilus
aacgcattaagcactccgcctggggagtacgaccgcaaggttgaaactcaaaggaattgacgggggcccgcacaagcggtggagcatgtggtttaattcgaagcaacgcgaagaaccttaccaggtcttga

SEQ ID NO: 57 - DNA, 69 bp, Lactobacillus acidophilus
aggtggtgcatggctgtcgtcagctcgtgtcgtgagatgttgggttaagtcccgcaacgagcgcaaccc

SEQ ID NO: 58 - DNA, 67 bp, Lactobacillus acidophilus
gaggaaggtggggatgacgtcaagtcatcatgccccttatgacctgggctacacacgtgctacaatg

SEQ ID NO: 59 - DNA, 110 bp, Lactobacillus acidophilus
agttcggactgcagtctgcaactcgactgcacgaagctggaatcgctagtaatcgcggatcagcacgccgcggtgaatacgttcccgggccttgtacacaccgcccgtca

SEQ ID NO: 60 - DNA, 89 bp, Lactobacillus acidophilus
ggaaggagccgtctaaggcagggcagatgactggggtgaagtcgtaacaaggtagccgtaggagaacctgcggctggatcacctccttt

SEQ ID NO: 61 - DNA, 1566 bp, Lactobacillus paracasei
tatgagagtttgatcctggctcaggatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctt

SEQ ID NO: 62 - DNA, 47 bp, Lactobacillus paracasei
agttctcgttgatgatcggtgcttgcaccgagattcaacatggaacg

SEQ ID NO: 63 - DNA, 71 bp, Lactobacillus paracasei
agatccaagaaccgcatggttcttggctgaaagatggcgtaagctatcgcttttggatggacccgcggcgt

SEQ ID NO: 64 - DNA, 63 bp, Lactobacillus paracasei; identical to SEQ ID NO: 24

SEQ ID NO: 65 - DNA, 85 bp, Lactobacillus paracasei
ttttttaagtctgatgtgaaagccctcggcttaaccgaggaagcgcatcggaaactgggaaacttgagtgcagaagaggacagtg

SEQ ID NO: 66 - DNA, 43 bp, Lactobacillus paracasei; identical to SEQ ID NO: 26

SEQ ID NO: 67 - DNA, 51 bp, Lactobacillus paracasei; identical to SEQ ID NO: 27

SEQ ID NO: 68 - DNA, 59 bp, Lactobacillus paracasei; identical to SEQ ID NO: 28

SEQ ID NO: 69 - DNA, 57 bp, Lactobacillus paracasei; identical to SEQ ID NO: 29

SEQ ID NO: 70 - DNA, 48 bp, Lactobacillus paracasei; identical to SEQ ID NO: 30

SEQ ID NO: 71 - DNA, 65 bp, Lactobacillus paracasei; identical to SEQ ID NO: 31

SEQ ID NO: 72 - DNA, 81 bp, Lactobacillus paracasei; identical to SEQ ID NO: 32

SEQ ID NO: 73 - DNA, 174 bp, Lactobacillus paracasei
attagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaag

SEQ ID NO: 74 - DNA, 109 bp, Lactobacillus paracasei; identical to SEQ ID NO: 34

SEQ ID NO: 75 - DNA, 148 bp, Lactobacillus paracasei; identical to SEQ ID NO: 35

SEQ ID NO: 76 - DNA, 131 bp, Lactobacillus paracasei; identical to SEQ ID NO: 36

SEQ ID NO: 77 - DNA, 69 bp, Lactobacillus paracasei; identical to SEQ ID NO: 17

SEQ ID NO: 78 - DNA, 67 bp, Lactobacillus paracasei; identical to SEQ ID NO: 18

SEQ ID NO: 79 - DNA, 110 bp, Lactobacillus paracasei; identical to SEQ ID NO: 39

SEQ ID NO: 80 - DNA, 88 bp, Lactobacillus paracasei; identical to SEQ ID NO: 40

SEQ ID NO: 81 - DNA, 1523 bp, Bifidobacterium longum
gtggagggttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 82 - DNA, 29 bp, Bifidobacterium longum
ggatccatcaggctttgcttggtggtgag

SEQ ID NO: 83 - DNA, 62 bp, Bifidobacterium longum
gctccagttgatcgcatggtcttctgggaaagctttcgcggtatgggatggggtcgcgtcct

SEQ ID NO: 84 - DNA, 43 bp, Bifidobacterium longum
gccttcgggttgtaaacctcttttatcggggagcaagcgagag

SEQ ID NO: 85 - DNA, 84 bp, Bifidobacterium longum
tcgtcgcgtccggtgtgaaagtccatcgcttaacggtggatccgcgccgggtacgggcgggcttgagtgcggtaggggagactg

SEQ ID NO: 86 - DNA, 44 bp, Bifidobacterium longum
gatgctggatgtggggcccgttccacgggttccgtgtcggagct

SEQ ID NO: 87 - DNA, 50 bp, Bifidobacterium longum
catgttcccgacggtcgtagagatacggcttcccttcggggcgggttcac

SEQ ID NO: 88 - DNA, 61 bp, Bifidobacterium longum
tcgccccgtgttgccagcggattatgccgggaactcacgggggaccgccggggttaactcg

SEQ ID NO: 89 - DNA, 56 bp, Bifidobacterium longum
gccggtacaacgggatgcgacgcggcgacgcggagcggatccctgaaaaccggtct

SEQ ID NO: 90 - DNA, 48 bp, Bifidobacterium longum
agtcatgaaagtgggcagcacccgaagccggtggcctaaccccttgtg

SEQ ID NO: 91 - DNA, 65 bp, Bifidobacterium longum
gtggagggttcgattctggctcaggatgaacgctggcggcgtgcttaacacatgcaagtcgaacg

SEQ ID NO: 92 - DNA, 81 bp, Bifidobacterium longum
agtggcgaacgggtgagtaatgcgtgaccgacctgccccatacaccggaatagctcctggaaacgggtggtaatgccggat

SEQ ID NO: 93 - DNA, 174 bp, Bifidobacterium longum
atcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggag

SEQ ID NO: 94 - DNA, 110 bp, Bifidobacterium longum
tgagtttacccgttgaataagcaccggctaactacgtgccagcagccgcggtaatacgtagggtgcaagcgttatccggaattattgggcgtaaagggctcgtaggcggt

SEQ ID NO: 95 - DNA, 148 bp, Bifidobacterium longum
gaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtg

SEQ ID NO: 96 - DNA, 131 bp, Bifidobacterium longum
aacgcgttaagcatcccgcctggggagtacggccgcaaggctaaaactcaaagaaattgacgggggcccgcacaagcggcggagcatgcggattaattcgatgcaacgcgaagaaccttacctgggcttga

SEQ ID NO: 97 - DNA, 69 bp, Bifidobacterium longum
aggtggtgcatggtcgtcgtcagctcgtgtcgtgagatgttgggttaagtcccgcaacgagcgcaaccc

SEQ ID NO: 98 - DNA, 67 bp, Bifidobacterium longum
gaggaaggtggggatgacgtcagatcatcatgccccttacgtccagggcttcacgcatgctacaatg

SEQ ID NO: 99 - DNA, 112 bp, Bifidobacterium longum
cagttcggatcgcagtctgcaactcgactgcgtgaaggcggagtcgctagtaatcgcgaatcagcaacgtcgcggtgaatgcgttcccgggccttgtacacaccgcccgtca

SEQ ID NO: 100 - DNA, 89 bp, Bifidobacterium longum
ggatggagccgtctaaggtgaggctcgtgattgggactaagtcgtaacaaggtagccgtaccggaaggtgcggctggatcacctccttt

SEQ ID NO: 101 - DNA, 1535 bp, Bifidobacterium animalis lactis
gtggagggttcgattctggctcaggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaaggtgagactcgtgattgggactaagtcgtaacaaggtagccgtaccggaaggtgcggctggatcacctccttt

SEQ ID NO: 102 - DNA, 30 bp, Bifidobacterium animalis lactis
ggatccctggcagcttgctgtcggggtgag

SEQ ID NO: 103 - DNA, 63 bp, Bifidobacterium animalis lactis
gctccgctccatcgcatggtggggtgggaaatgcttttgcggcatgggatggggtcgcgtcct

SEQ ID NO: 104 - DNA, 62 bp, Bifidobacterium animalis lactis
gccttcgggttgtaaaccgcttttgttcaagggcaaggcacggtttcggccgtgttgagtgg

SEQ ID NO: 105 - DNA, 85 bp, Bifidobacterium animalis lactis
ttcgtcgcgtccggtgtgaaagtccatcgcctaacggtggatctgcgccgggtacgggcgggctggagtgcggtaggggagactg

SEQ ID NO: 106 - DNA, 44 bp, Bifidobacterium animalis lactis
gatgctggatgtggggccctttccacgggtcccgtgtcggagcc

SEQ ID NO: 107 - DNA, 50 bp, Bifidobacterium animalis lactis
catgtgccggatcgccgtggagacacggtttcccttcggggccggttcac

SEQ ID NO: 108 - DNA, 61 bp, Bifidobacterium animalis lactis
tcgccgcatgttgccagcgggtgatgccgggaactcatgtgggaccgccggggtcaactcg

SEQ ID NO: 109 - DNA, 57 bp, Bifidobacterium animalis lactis
gccggtacaacgcggtgcgacacggtgacgtggggcggatcgctgaaaaccggtctc

SEQ ID NO: 110 - DNA, 46 bp, Bifidobacterium animalis lactis
agtcatgaaagtgggtagcacccgaagccggtggcccgacccttgt

SEQ ID NO: 111 - DNA, 65 bp, Bifidobacterium animalis lactis; identical to SEQ ID NO: 91

SEQ ID NO: 112 - DNA, 81 bp, Bifidobacterium animalis lactis
agtggcgaacgggtgagtaatgcgtgaccaacctgccctgtgcaccggaatagctcctggaaacgggtggtaataccggat

SEQ ID NO: 113 - DNA, 174 bp, Bifidobacterium animalis lactis
atcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggag

SEQ ID NO: 114 - DNA, 102 bp, Bifidobacterium animalis lactis
attgttcgaataagcaccggctaactacgtgccagcagccgcggtaatacgtagggtgcgagcgttatccggatttattgggcgtaaagggctcgtaggcgg

SEQ ID NO: 115 - DNA, 148 bp, Bifidobacterium animalis lactis
gaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtg

SEQ ID NO: 116 - DNA, 131 bp, Bifidobacterium animalis lactis; identical to SEQ ID NO: 96

SEQ ID NO: 117 - DNA, 69 bp, Bifidobacterium animalis lactis; identical to SEQ ID NO: 97

SEQ ID NO: 118 - DNA, 67 bp, Bifidobacterium animalis lactis; identical to SEQ ID NO: 98

SEQ ID NO: 119 - DNA, 111 bp, Bifidobacterium animalis lactis
agttcggatcgcagtctgcaactcgactgcgtgaaggcggagtcgctagtaatcgcggatcagcaacgccgcggtgaatgcgttcccgggccttgtacacaccgcccgtca

SEQ ID NO: 120 - DNA, 89 bp, Bifidobacterium animalis lactis
ggggggagccgtctaaggtgagactcgtgattgggactaagtcgtaacaaggtagccgtaccggaaggtgcggctggatcacctccttt

SEQ ID NO: 121 - DNA, 1560 bp, Veillonella dispar
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggatggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagttagaaggattgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcaataccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 122 - DNA, 32 bp, Veillonella dispar
aagagcgatggaagcttgcttctatcaatctt

SEQ ID NO: 123 - DNA, 80 bp, Veillonella dispar
acgatctaacctcggcatcgaggatggatgaaaggtggcctctatttataagctatcactgaaggaggggattgcgtctg

SEQ ID NO: 124 - DNA, 64 bp, Veillonella dispar
gccttcgggttgtaaagctctgttaatcgggacgaaaggccttcttgcgaatagttagaaggat

SEQ ID NO: 125 - DNA, 84 bp, Veillonella dispar
attggtcagtctgtcttaaaagttcggggcttaaccccgtgatgggatggaaactgccaatctagagtatcggagaggaaagtg

SEQ ID NO: 126 - DNA, 43 bp, Veillonella dispar
ggtactaggtgtaggaggtatcgaccccttctgtgccggagtt

SEQ ID NO: 127 - DNA, 51 bp, Veillonella dispar
cattgatggacagaactagagatagttcctcttcttcggaagccagaaaac

SEQ ID NO: 128 - DNA, 60 bp, Veillonella dispar
ctatcttatgttgccagcacttcgggtgggaactcatgagagactgccgcagacaatgcg

SEQ ID NO: 129 - DNA, 58 bp, Veillonella dispar
ggagttaatagacggaagcaataccgcgaggtggagcaaacccgagaaacactctctc

SEQ ID NO: 130 - DNA, 45 bp, Veillonella dispar
caccacgaaagtcggaagtgcccaaagccggtggggtaaccttcg

SEQ ID NO: 131 - DNA, 65 bp, Veillonella dispar
ttggagagtttgatcctggctcaggacgaacgctggcggcgtgcttaacacatgcaagtcgaacg

SEQ ID NO: 132 - DNA, 81 bp, Veillonella dispar
agtggcgaacgggtgagtaacgcgtaatcaacctgcccttcagagggggacaacagttggaaacgactgctaataccgcat

SEQ ID NO: 133 - DNA, 174 bp, Veillonella dispar
attagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacg

SEQ ID NO: 134 - DNA, 109 bp, Veillonella dispar
tgacggtaccggaatagaaagccacggctaactacgtgccagcagccgcggtaatacgtaggtggcaagcgttgtccggaattattgggcgtaaagcgcgcgcaggcgg

SEQ ID NO: 135 - DNA, 148 bp, Veillonella dispar
gaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatg

SEQ ID NO: 136 - DNA, 131 bp, Veillonella dispar
aacgcaataagtaccccgcctggggagtacgaccgcaaggttgaaactcaaaggaattgacgggggcccgcacaagcggtggagtatgtggtttaattcgacgcaacgcgaagaaccttaccaggtcttga

SEQ ID NO: 137 - DNA, 69 bp, Veillonella dispar
aggtggtgcacggttgtcgtcagctcgtgtcgtgagatgttgggttaagtcccgcaacgagcgcaaccc

SEQ ID NO: 138 - DNA, 67 bp, Veillonella dispar
gaggaaggcggggatgacgtcaaatcatcatgccccttatgacctgggctacacacgtactacaatg

SEQ ID NO: 139 - DNA, 110 bp, Veillonella dispar
agttcggatcgtaggctgcaactcgcctacgtgaagtcggaatcgctagtaatcgcaggtcagcatactgcggtgaatacgttcccgggccttgtacacaccgcccgtca

SEQ ID NO: 140 - DNA, 89 bp, Veillonella dispar
ggagccagccgtctaaggtaaagtcgatgattggggtgaagtcgtaacaaggtagccgtatcggaaggtgcggctggatcacctccttt

SEQ ID NO: 141 - DNA, 1560 bp, Veillonella parvula
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggaaagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtggggactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 142 - DNA, 32 bp, Veillonella parvula; identical to SEQ ID NO: 122

SEQ ID NO: 143 - DNA, 80 bp, Veillonella parvula
acgatctaatctcggcatcgaggaaagatgaaaggtggcctctatttataagctatcactgaaggaggggattgcgtctg

SEQ ID NO: 144 - DNA, 64 bp, Veillonella parvula
gccttcgggttgtaaagctctgttaatcgggacgaaaggccttcttgcgaacagttagaaggat

SEQ ID NO: 145 - DNA, 84 bp, Veillonella parvula
atcagtcagtctgtcttaaaagttcggggcttaaccccgtgatgggatggaaactgctgatctagagtatcggagaggaaagtg

SEQ ID NO: 146 - DNA, 43 bp, Veillonella parvula; identical to SEQ ID NO: 126

SEQ ID NO: 147 - DNA, 51 bp, Veillonella parvula
cattgatggacagaaccagagatggttcctcttcttcggaagccagaaaac

SEQ ID NO: 148 - DNA, 60 bp, Veillonella parvula
ctatcttatgttgccagcactttgggtggggactcatgagagactgccgcagacaatgcg

SEQ ID NO: 149 - DNA, 58 bp, Veillonella parvula
ggagttaatagacggaagcgagatcgcgagatggagcaaacccgagaaacactctctc

SEQ ID NO: 150 - DNA, 45 bp, Veillonella parvula; identical to SEQ ID NO: 130

SEQ ID NO: 151 - DNA, 65 bp, Veillonella parvula; identical to SEQ ID NO: 131

SEQ ID NO: 152 - DNA, 81 bp, Veillonella parvula; identical to SEQ ID NO: 132

SEQ ID NO: 153 - DNA, 174 bp, Veillonella parvula; identical to SEQ ID NO: 133

SEQ ID NO: 154 - DNA, 109 bp, Veillonella parvula; identical to SEQ ID NO: 134

SEQ ID NO: 155 - DNA, 148 bp, Veillonella parvula; identical to SEQ ID NO: 135

SEQ ID NO: 156 - DNA, 131 bp, Veillonella parvula; identical to SEQ ID NO: 136

SEQ ID NO: 157 - DNA, 69 bp, Veillonella parvula; identical to SEQ ID NO: 137

SEQ ID NO: 158 - DNA, 67 bp, Veillonella parvula; identical to SEQ ID NO: 138

SEQ ID NO: 159 - DNA, 110 bp, Veillonella parvula; identical to SEQ ID NO: 139

SEQ ID NO: 160 - DNA, 89 bp, Veillonella parvula; identical to SEQ ID NO: 140

SEQ ID NO: 161 - DNA, 1560 bp, Veillonella atypica
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatccaatctcggcatcgagactggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcgaaaccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 162 - DNA, 32 bp, Veillonella atypica; identical to SEQ ID NO: 122

SEQ ID NO: 163 - DNA, 80 bp, Veillonella atypica
acgatccaatctcggcatcgagactggatgaaaggtggcctctatttataagctatcactgaaggaggggattgcgtctg

SEQ ID NO: 164 - DNA, 64 bp, Veillonella atypica
gccttcgggttgtaaagctctgttaatcgggacgaatggttcttgtgcgaatagtgcgaggatt

SEQ ID NO: 165 - DNA, 84 bp, Veillonella atypica; identical to SEQ ID NO: 125

SEQ ID NO: 166 - DNA, 43 bp, Veillonella atypica; identical to SEQ ID NO: 126

SEQ ID NO: 167 - DNA, 51 bp, Veillonella atypica; identical to SEQ ID NO: 127

SEQ ID NO: 168 - DNA, 60 bp, Veillonella atypica; identical to SEQ ID NO: 128

SEQ ID NO: 169 - DNA, 58 bp, Veillonella atypica
ggagttaatagacggaagcgaaaccgcgaggtggagcaaacccgagaaacactctctc

SEQ ID NO: 170 - DNA, 45 bp, Veillonella atypica; identical to SEQ ID NO: 130

SEQ ID NO: 171 - DNA, 65 bp, Veillonella atypica; identical to SEQ ID NO: 131

SEQ ID NO: 172 - DNA, 81 bp, Veillonella atypica; identical to SEQ ID NO: 132

SEQ ID NO: 173 - DNA, 174 bp, Veillonella atypica; identical to SEQ ID NO: 133

SEQ ID NO: 174 - DNA, 109 bp, Veillonella atypica; identical to SEQ ID NO: 134

SEQ ID NO: 175 - DNA, 148 bp, Veillonella atypica; identical to SEQ ID NO: 135

SEQ ID NO: 176 - DNA, 131 bp, Veillonella atypica; identical to SEQ ID NO: 136

SEQ ID NO: 177 - DNA, 69 bp, Veillonella atypica; identical to SEQ ID NO: 137

SEQ ID NO: 178 - DNA, 67 bp, Veillonella atypica; identical to SEQ ID NO: 138

SEQ ID NO: 179 - DNA, 110 bp, Veillonella atypica; identical to SEQ ID NO: 139

SEQ ID NO: 180 - DNA, 89 bp, Veillonella atypica; identical to SEQ ID NO: 140

SEQ ID NO: 181 - DNA, 1566 bp, Lactobacillus paracasei; identical to SEQ ID NO: 61

SEQ ID NO: 182 - DNA, 47 bp, Lactobacillus paracasei; identical to SEQ ID NO: 62

SEQ ID NO: 183 - DNA, 71 bp, Lactobacillus paracasei; identical to SEQ ID NO: 63

SEQ ID NO: 184 - DNA, 63 bp, Lactobacillus paracasei; identical to SEQ ID NO: 24

SEQ ID NO: 185 - DNA, 85 bp, Lactobacillus paracasei; identical to SEQ ID NO: 65

SEQ ID NO: 186 - DNA, 43 bp, Lactobacillus paracasei; identical to SEQ ID NO: 26

SEQ ID NO: 187 - DNA, 51 bp, Lactobacillus paracasei; identical to SEQ ID NO: 27

SEQ ID NO: 188 - DNA, 59 bp, Lactobacillus paracasei; identical to SEQ ID NO: 28

SEQ ID NO: 189 - DNA, 57 bp, Lactobacillus paracasei; identical to SEQ ID NO: 29

SEQ ID NO: 190 - DNA, 48 bp, Lactobacillus paracasei; identical to SEQ ID NO: 30

SEQ ID NO: 191 - DNA, 65 bp, Lactobacillus paracasei; identical to SEQ ID NO: 31

SEQ ID NO: 192 - DNA, 81 bp, Lactobacillus paracasei; identical to SEQ ID NO: 32

SEQ ID NO: 193 - DNA, 174 bp, Lactobacillus paracasei; identical to SEQ ID NO: 73

SEQ ID NO: 194 - DNA, 109 bp, Lactobacillus paracasei; identical to SEQ ID NO: 34

SEQ ID NO: 195 - DNA, 148 bp, Lactobacillus paracasei; identical to SEQ ID NO: 35

SEQ ID NO: 196 - DNA, 131 bp, Lactobacillus paracasei; identical to SEQ ID NO: 36

SEQ ID NO: 197 - DNA, 69 bp, Lactobacillus paracasei; identical to SEQ ID NO: 17

SEQ ID NO: 198 - DNA, 67 bp, Lactobacillus paracasei; identical to SEQ ID NO: 18

SEQ ID NO: 199 - DNA, 110 bp, Lactobacillus paracasei; identical to SEQ ID NO: 39

SEQ ID NO: 200 - DNA, 88 bp, Lactobacillus paracasei; identical to SEQ ID NO: 40

SEQ ID NO: 201 - DNA, 1524 bp, Bifidobacterium longum
tgtggagggttcgattctggctcaggatgaacgctggcggcgtgcttaacacatgcaagtcgaacgggatccaccgggctttgcctggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 202 - DNA, 29 bp, Bifidobacterium longum
ggatccaccgggctttgcctggtggtgag

SEQ ID NO: 203 - DNA, 62 bp, Bifidobacterium longum; identical to SEQ ID NO: 83

SEQ ID NO: 204 - DNA, 43 bp, Bifidobacterium longum; identical to SEQ ID NO: 84

SEQ ID NO: 205 - DNA, 85 bp, Bifidobacterium longum
ttcgtcgcgtccggtgtgaaagtccatcgcttaacggtggatccgcgccgggtacgggcgggcttgagtgcggtaggggagactg

SEQ ID NO: 206 - DNA, 44 bp, Bifidobacterium longum; identical to SEQ ID NO: 86

SEQ ID NO: 207 - DNA, 50 bp, Bifidobacterium longum; identical to SEQ ID NO: 87

SEQ ID NO: 208 - DNA, 61 bp, Bifidobacterium longum; identical to SEQ ID NO: 88

SEQ ID NO: 209 - DNA, 57 bp, Bifidobacterium longum
gccggtacaacgggatgcgacgcggcgacgcggagcggatccctgaaaaccggtctc

SEQ ID NO: 210 - DNA, 48 bp, Bifidobacterium longum; identical to SEQ ID NO: 90

SEQ ID NO: 211 - DNA, 66 bp, Bifidobacterium longum
tgtggagggttcgattctggctcaggatgaacgctggcggcgtgcttaacacatgcaagtcgaacg

SEQ ID NO: 212 - DNA, 81 bp, Bifidobacterium longum; identical to SEQ ID NO: 92

SEQ ID NO: 213 - DNA, 174 bp, Bifidobacterium longum; identical to SEQ ID NO: 93

SEQ ID NO: 214 - DNA, 109 bp, Bifidobacterium longum
tgagtttacccgttgaataagcaccggctaactacgtgccagcagccgcggtaatacgtagggtgcaagcgttatccggaattattgggcgtaaagggctcgtaggcgg

SEQ ID NO: 215 - DNA, 148 bp, Bifidobacterium longum; identical to SEQ ID NO: 95

SEQ ID NO: 216 - DNA, 131 bp, Bifidobacterium longum; identical to SEQ ID NO: 96

SEQ ID NO: 217 - DNA, 69 bp, Bifidobacterium longum; identical to SEQ ID NO: 97

SEQ ID NO: 218 - DNA, 67 bp, Bifidobacterium longum; identical to SEQ ID NO: 98

SEQ ID NO: 219 - DNA, 111 bp, Bifidobacterium longum
agttcggatcgcagtctgcaactcgactgcgtgaaggcggagtcgctagtaatcgcgaatcagcaacgtcgcggtgaatgcgttcccgggccttgtacacaccgcccgtca

SEQ ID NO: 220 - DNA, 89 bp, Bifidobacterium longum; identical to SEQ ID NO: 100

SEQ ID NO: 221 - DNA, 1523 bp, Bifidobacterium longum
gtggagggttcgattctggctcaggatgaacgctggcggcgtgcttaacacatgcaagtcgaacgggatccatcgggctttgcctggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatgcggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 222 - DNA, 29 bp, Bifidobacterium longum
ggatccatcgggctttgcctggtggtgag

SEQ ID NO: 223 - DNA, 62 bp, Bifidobacterium longum; identical to SEQ ID NO: 83

SEQ ID NO: 224 - DNA, 43 bp, Bifidobacterium longum; identical to SEQ ID NO: 84

SEQ ID NO: 225 - DNA, 85 bp, Bifidobacterium longum; identical to SEQ ID NO: 205

SEQ ID NO: 226 - DNA, 44 bp, Bifidobacterium longum; identical to SEQ ID NO: 86

SEQ ID NO: 227 - DNA, 50 bp, Bifidobacterium longum
catgttcccgacggtcgtagagatgcggcttcccttcggggcgggttcac

SEQ ID NO: 228 - DNA, 61 bp, Bifidobacterium longum; identical to SEQ ID NO: 88

SEQ ID NO: 229 - DNA, 57 bp, Bifidobacterium longum; identical to SEQ ID NO: 209

SEQ ID NO: 230 - DNA, 48 bp, Bifidobacterium longum; identical to SEQ ID NO: 90

SEQ ID NO: 231 - DNA, 65 bp, Bifidobacterium longum; identical to SEQ ID NO: 91

SEQ ID NO: 232 - DNA, 81 bp, Bifidobacterium longum; identical to SEQ ID NO: 92

SEQ ID NO: 233 - DNA, 174 bp, Bifidobacterium longum; identical to SEQ ID NO: 93

SEQ ID NO: 234 - DNA, 109 bp, Bifidobacterium longum; identical to SEQ ID NO: 214

SEQ ID NO: 235 - DNA, 148 bp, Bifidobacterium longum; identical to SEQ ID NO: 95

SEQ ID NO: 236 - DNA, 131 bp, Bifidobacterium longum; identical to SEQ ID NO: 96

SEQ ID NO: 237 - DNA, 69 bp, Bifidobacterium longum; identical to SEQ ID NO: 97

SEQ ID NO: 238 - DNA, 67 bp, Bifidobacterium longum; identical to SEQ ID NO: 98

SEQ ID NO: 239 - DNA, 111 bp, Bifidobacterium longum; identical to SEQ ID NO: 219

SEQ ID NO: 240 - DNA, 89 bp, Bifidobacterium longum; identical to SEQ ID NO: 100

(19) United States
(12) Patent Application Publication
(10) Pub. No.: US 2023/0112367 A1
(43) Pub. Date: Apr. 13, 2023

(54) PROBIOTIC COMPOSITIONS FOR IMPROVING HUMAN HEALTH AND ATHLETIC PERFORMANCE

(71) Applicant: FITBIOMICS INC., New York, NY (US)

(72) Inventor: Jonathan SCHEIMAN, New York, NY (US)

(21) Appl. No.: 17/911,255

(22) PCT Filed: Mar. 15, 2021
(86) PCT No.: PCT/US2021/022380

§ 371 Date: Sep. 13, 2022

Related U.S. Application Data
(60) Provisional application No. 63/018,697, filed on May 1, 2020.
(60) Provisional application No. 62/989,226, filed on Mar. 13, 2020.

(30) Foreign Application Priority Data
Nov. 25, 2020 (US) PCT/US2020/062336

(51) Int. Cl. A61K 35/747; A61K 35/745; C12N 1/20

(52) U.S. Cl. CPC A61K 35/747; A61K 35/745; C12N 1/205; A61K 2035/115

(57) ABSTRACT

The disclosure provides novel microorganisms, such as Lactobacillus sp., Bifidobacterium sp., and Veillonella sp., and compositions thereof for improving athletic performance, enhancing exercise endurance, improving physical recovery from exercise, and reducing inflammation in a subject in need thereof.

### CROSS-REFERENCE TO RELATED APPLICATIONS

This application claims the benefit of priority to U.S. Provisional Application No. 62/989,226, filed on Mar. 13, 2020, U.S. Provisional Application No. 63/018,697, filed on May 1, 2020, and PCT Application No. PCT/US2020/062336, filed on Nov. 25, 2020; each of which are herein incorporated by reference in their entireties.

### TECHNICAL FIELD

The present disclosure relates to compositions comprising one or more microbial strains, and methods of use thereof in improving athletic performance.

### INCORPORATION OF SEQUENCE LISTING

The contents of the text file submitted electronically herewith are incorporated by reference in their entirety: a computer readable format copy of the Sequence Listing (filename: “FIBI_002_02WO_SeqList_ST25”, date recorded: Mar. 12, 2021, file size: 75.7 kilobytes).

### BACKGROUND

Human gastrointestinal microbiota, also known as gut flora or gut microbiota, comprise microorganisms that live in the digestive tracts of humans, and play a critical role in human health. These microorganisms serve a wide range of functions, such as defending against pathogens, fortifying host defense by developing and maintaining the intestinal epithelium, inducing antibody production, metabolizing indigestible compounds in food, and training the developing immune system. The gut microbiome also plays a role in the biochemical signaling between the gastrointestinal tract and the central nervous system, referred to as the gut-brain axis.

Formulations comprising microorganisms are used as dietary supplements, and are deemed to provide health benefits by restoring or improving the gut microflora. These microbes may be either resident or transient; resident microbial strains live and reproduce in the digestive tract, while transient microbial strains are introduced into the body through ingested food or by means of dietary supplements. Formulations comprising microorganisms are thought to have immense potential in improving human health.

Formulations comprising microorganisms may have unexplored potential in improving other human functions, such as endurance and athleticism. Pharmaceuticals that improve endurance and athleticism are in popular use. However, there remains a need to develop compositions comprising microorganisms that helps improve endurance and athleticism, for instance, promote or accelerate athletic training, performance and recovery.

### SUMMARY

The present disclosure provides an isolated and purified Lactobacillus plantarum, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 2; (ii) VR2 comprises a nucleic acid sequence with at least 95% sequence identity to SEQ ID NO: 3; (iii) VR3 comprises a nucleic acid sequence with at least 95% sequence identity to SEQ ID NO: 4; (iv) VR4 comprises a nucleic acid sequence with at least 95% sequence identity to SEQ ID NO: 5; (v) VR5 comprises a nucleic acid sequence with at least 95% sequence identity to SEQ ID NO: 6; (vi) VR6 comprises a nucleic acid sequence with at least 95% sequence identity to SEQ ID NO: 7; (vii) VR7 comprises a nucleic acid sequence with at least 95% sequence identity to SEQ ID NO: 8; (viii) VR8 comprises a nucleic acid sequence with at least 95% sequence identity to SEQ ID NO: 9; and (ix) VR9 comprises a nucleic acid sequence with at least 95% sequence identity to SEQ ID NO: 10.

In some embodiments, the Lactobacillus plantarum comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 11; (ii) CR2 comprises a nucleic acid sequence with at least 80% sequence identity to SEQ ID NO: 12; (iii) CR3 comprises a nucleic acid sequence with at least 80% sequence identity to SEQ ID NO: 13; (iv) CR4 comprises a nucleic acid sequence with at least 80% sequence identity to SEQ ID NO:14; (v) CR5 comprises a nucleic acid sequence with at least 80% sequence identity to SEQ ID NO: 15; (vi) CR6 comprises a nucleic acid sequence with at least 80% sequence identity to SEQ ID NO: 16; (vii) CR7 comprises a nucleic acid sequence with at least 80% sequence identity to SEQ ID NO: 17; (viii) CR8 comprises a nucleic acid sequence with at least 80% sequence identity to SEQ ID NO: 18; (ix) CR9 comprises a nucleic acid sequence with at least 80% sequence identity to SEQ ID NO: 19; and (x) CR10 comprises a nucleic acid sequence with at least 80% sequence identity to SEQ ID NO: 20.

In some embodiments, the present disclosure provides an isolated and purified Lactobacillus plantarum, comprising: a 16S rRNA gene comprising a nucleic acid sequence with at least 97% sequence identity to SEQ ID NO: 1.

In some embodiments, the present disclosure provides an isolated and purified Lactobacillus plantarum having deposit accession number 202103009, or a strain having all of the identifying characteristics of Lactobacillus plantarum 202103009, or a mutant thereof.

In some embodiments, the present disclosure provides an isolated and purified Lactobacillus rhamnosus, comprising: a 16S rRNA gene comprising a nucleic acid sequence comprising at least one variable region (VR) selected from the group consisting of VR1, VR2, VR3, VR4, VR5, VR6, VR7, VR8, VR9, and any combination thereof, wherein (i) VR1 comprises a nucleic acid sequence with at least 95% sequence identity to SEQ ID NO: 22; (ii) VR2 comprises a nucleic acid sequence with at least 95% sequence identity to SEQ ID NO: 23; (iii) VR3 comprises a nucleic acid sequence with at least 95% sequence identity to SEQ ID NO: 24; (iv) VR4 comprises a nucleic acid sequence with at least 95% sequence identity to SEQ ID NO: 25; (v) VR5 comprises a nucleic acid sequence with at least 95% sequence identity to SEQ ID NO: 26; (vi) VR6 comprises a nucleic acid sequence with at least 95% sequence identity to SEQ ID NO: 27; (vii) VR7 comprises a nucleic acid sequence with at least 95% sequence identity to SEQ ID NO: 28; (viii) VR8 comprises a nucleic acid sequence with at least 95% sequence identity to SEQ ID NO: 29; and (ix) VR9 comprises a nucleic acid sequence with at least 95% sequence identity to SEQ ID NO: 30.

In some embodiments, the Lactobacillus rhamnosus comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 31; (ii) CR2 comprises a nucleic acid sequence with at least 80% sequence identity to SEQ ID NO: 32; (iii) CR3 comprises a nucleic acid sequence with at least 80% sequence identity to SEQ ID NO: 33; (iv) CR4 comprises a nucleic acid sequence with at least 80% sequence identity to SEQ ID NO: 34; (v) CR5 comprises a nucleic acid sequence with at least 80% sequence identity to SEQ ID NO: 35; (vi) CR6 comprises a nucleic acid sequence with at least 80% sequence identity to SEQ ID NO: 36; (vii) CR7 comprises a nucleic acid sequence with at least 80% sequence identity to SEQ ID NO: 37; (viii) CR8 comprises a nucleic acid sequence with at least 80% sequence identity to SEQ ID NO: 38; (ix) CR9 comprises a nucleic acid sequence with at least 80% sequence identity to SEQ ID NO: 39; and (x) CR10 comprises a nucleic acid sequence with at least 80% sequence identity to SEQ ID NO: 40.

In some embodiments, the present disclosure provides an isolated and purified Lactobacillus rhamnosus, comprising: a 16S rRNA gene comprising a nucleic acid sequence with at least 97% sequence identity to SEQ ID NO: 21.

In some embodiments, the present disclosure provides an isolated and purified Lactobacillus rhamnosus having deposit accession number 202103010, or a strain having all of the identifying characteristics of Lactobacillus rhamnosus 202103010, or a mutant thereof.

In some embodiments, the present disclosure provides an isolated and purified Lactobacillus acidophilus, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 42; (ii) VR2 comprises a nucleic acid sequence with at least 95% sequence identity to SEQ ID NO: 43; (iii) VR3 comprises a nucleic acid sequence with at least 95% sequence identity to SEQ ID NO: 44; (iv) VR4 comprises a nucleic acid sequence with at least 95% sequence identity to SEQ ID NO: 45; (v) VR5 comprises a nucleic acid sequence with at least 95% sequence identity to SEQ ID NO: 46; (vi) VR6 comprises a nucleic acid sequence with at least 95% sequence identity to SEQ ID NO: 47; (vii) VR7 comprises a nucleic acid sequence with at least 95% sequence identity to SEQ ID NO: 48; (viii) VR8 comprises a nucleic acid sequence with at least 95% sequence identity to SEQ ID NO: 49; and (ix) VR9 comprises a nucleic acid sequence with at least 95% sequence identity to SEQ ID NO: 50.

In some embodiments, the Lactobacillus acidophilus comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 51; (ii) CR2 comprises a nucleic acid sequence with at least 80% sequence identity to SEQ ID NO: 52; (iii) CR3 comprises a nucleic acid sequence with at least 80% sequence identity to SEQ ID NO: 53; (iv) CR4 comprises a nucleic acid sequence with at least 80% sequence identity to SEQ ID NO: 54; (v) CR5 comprises a nucleic acid sequence with at least 80% sequence identity to SEQ ID NO: 55; (vi) CR6 comprises a nucleic acid sequence with at least 80% sequence identity to SEQ ID NO: 56; (vii) CR7 comprises a nucleic acid sequence with at least 80% sequence identity to SEQ ID NO: 57; (viii) CR8 comprises a nucleic acid sequence with at least 80% sequence identity to SEQ ID NO: 58; (ix) CR9 comprises a nucleic acid sequence with at least 80% sequence identity to SEQ ID NO: 59; and (x) CR10 comprises a nucleic acid sequence with at least 80% sequence identity to SEQ ID NO: 60.

In some embodiments, the present disclosure provides an isolated and purified Lactobacillus acidophilus, comprising: a 16S rRNA gene comprising a nucleic acid sequence with at least 97% sequence identity to SEQ ID NO: 41.

In some embodiments, the present disclosure provides an isolated and purified Lactobacillus acidophilus having deposit accession number 202103008, or a strain having all of the identifying characteristics of Lactobacillus acidophilus 202103008, or a mutant thereof.

In some embodiments, the present disclosure provides an isolated and purified Lactobacillus paracasei, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 62 or SEQ ID NO: 182; (ii) VR2 comprises a nucleic acid sequence with at least 95% sequence identity to SEQ ID NO: 63 or SEQ ID NO: 183; (iii) VR3 comprises a nucleic acid sequence with at least 95% sequence identity to SEQ ID NO: 64 or SEQ ID NO: 184; (iv) VR4 comprises a nucleic acid sequence with at least 95% sequence identity to SEQ ID NO: 65 or SEQ ID NO: 185; (v) VR5 comprises a nucleic acid sequence with at least 95% sequence identity to SEQ ID NO: 66 or SEQ ID NO: 186; (vi) VR6 comprises a nucleic acid sequence with at least 95% sequence identity to SEQ ID NO: 67 or SEQ ID NO: 187; (vii) VR7 comprises a nucleic acid sequence with at least 95% sequence identity to SEQ ID NO: 68 or SEQ ID NO: 188; (viii) VR8 comprises a nucleic acid sequence with at least 95% sequence identity to SEQ ID NO: 69 or SEQ ID NO: 189; and (ix) VR9 comprises a nucleic acid sequence with at least 95% sequence identity to SEQ ID NO: 70 or SEQ ID NO: 190.

In some embodiments, the Lactobacillus paracasei comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 71 or SEQ ID NO: 191; (ii) CR2 comprises a nucleic acid sequence with at least 80% sequence identity to SEQ ID NO: 72 or SEQ ID NO: 192; (iii) CR3 comprises a nucleic acid sequence with at least 80% sequence identity to SEQ ID NO: 73 or SEQ ID NO: 193; (iv) CR4 comprises a nucleic acid sequence with at least 80% sequence identity to SEQ ID NO: 74 or SEQ ID NO: 194; (v) CR5 comprises a nucleic acid sequence with at least 80% sequence identity to SEQ ID NO: 75 or SEQ ID NO: 195; (vi) CR6 comprises a nucleic acid sequence with at least 80% sequence identity to SEQ ID NO: 76 or SEQ ID NO: 196; (vii) CR7 comprises a nucleic acid sequence with at least 80% sequence identity to SEQ ID NO: 77 or SEQ ID NO: 197; (viii) CR8 comprises a nucleic acid sequence with at least 80% sequence identity to SEQ ID NO: 78 or SEQ ID NO: 198; (ix) CR9 comprises a nucleic acid sequence with at least 80% sequence identity to SEQ ID NO: 79 or SEQ ID NO: 199; and (x) CR10 comprises a nucleic acid sequence with at least 80% sequence identity to SEQ ID NO: 80 or SEQ ID NO: 200.

In some embodiments, the present disclosure provides an isolated and purified Lactobacillus paracasei, comprising: a 16S rRNA gene comprising a nucleic acid sequence with at least 97% sequence identity to SEQ ID NO: 61 or SEQ ID NO: 181.

In some embodiments, the present disclosure provides an isolated and purified Lactobacillus paracasei having deposit accession number 202103012 or 202103018, or a strain having all of the identifying characteristics of Lactobacillus paracasei 202103012 or 202103018, or a mutant thereof.

In some embodiments, the present disclosure provides an isolated and purified Bifidobacterium longum, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 82, SEQ ID NO: 202, or SEQ ID NO: 222; (ii) VR2 comprises a nucleic acid sequence with at least 95% sequence identity to SEQ ID NO: 83, SEQ ID NO: 203, or SEQ ID NO: 223; (iii) VR3 comprises a nucleic acid sequence with at least 95% sequence identity to SEQ ID NO: 84, SEQ ID NO: 204, or SEQ ID NO: 224; (iv) VR4 comprises a nucleic acid sequence with at least 95% sequence identity to SEQ ID NO: 85, SEQ ID NO: 205, or SEQ ID NO: 225; (v) VR5 comprises a nucleic acid sequence with at least 95% sequence identity to SEQ ID NO: 86, SEQ ID NO: 206, or SEQ ID NO: 226; (vi) VR6 comprises a nucleic acid sequence with at least 95% sequence identity to SEQ ID NO: 87, SEQ ID NO: 207, or SEQ ID NO: 227; (vii) VR7 comprises a nucleic acid sequence with at least 95% sequence identity to SEQ ID NO: 88, SEQ ID NO: 208, or SEQ ID NO: 228; (viii) VR8 comprises a nucleic acid sequence with at least 95% sequence identity to SEQ ID NO: 89, SEQ ID NO: 209, or SEQ ID NO: 229; and (ix) VR9 comprises a nucleic acid sequence with at least 95% sequence identity to SEQ ID NO: 90, SEQ ID NO: 210, or SEQ ID NO: 230.

In some embodiments, the Bifidobacterium longum comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 91, SEQ ID NO: 211, or SEQ ID NO: 231; (ii) CR2 comprises a nucleic acid sequence with at least 80% sequence identity to SEQ ID NO: 92, SEQ ID NO: 212, or SEQ ID NO: 232; (iii) CR3 comprises a nucleic acid sequence with at least 80% sequence identity to SEQ ID NO: 93, SEQ ID NO: 213, or SEQ ID NO: 233; (iv) CR4 comprises a nucleic acid sequence with at least 80% sequence identity to SEQ ID NO: 94, SEQ ID NO: 214, or SEQ ID NO: 234; (v) CR5 comprises a nucleic acid sequence with at least 80% sequence identity to SEQ ID NO: 95, SEQ ID NO: 215, or SEQ ID NO: 235; (vi) CR6 comprises a nucleic acid sequence with at least 80% sequence identity to SEQ ID NO: 96, SEQ ID NO: 216, or SEQ ID NO: 236; (vii) CR7 comprises a nucleic acid sequence with at least 80% sequence identity to SEQ ID NO: 97, SEQ ID NO: 217, or SEQ ID NO: 237; (viii) CR8 comprises a nucleic acid sequence with at least 80% sequence identity to SEQ ID NO: 98, SEQ ID NO: 218, or SEQ ID NO: 238; (ix) CR9 comprises a nucleic acid sequence with at least 80% sequence identity to SEQ ID NO: 99, SEQ ID NO: 219, or SEQ ID NO: 239; and (x) CR10 comprises a nucleic acid sequence with at least 80% sequence identity to SEQ ID NO: 100, SEQ ID NO: 220, or SEQ ID NO: 240.

In some embodiments, the present disclosure provides an isolated and purified Bifidobacterium longum, comprising: a 16S rRNA gene comprising a nucleic acid sequence with at least 97% sequence identity to SEQ ID NO: 81, SEQ ID NO: 201, or SEQ ID NO: 221.

In some embodiments, the present disclosure provides an isolated and purified Bifidobacterium longum having deposit accession number 202103013, 202103014, or 202103015, or a strain having all of the identifying characteristics of Bifidobacterium longum 202103013, 202103014, or 202103015, or a mutant thereof.

In some embodiments, the present disclosure provides an isolated and purified Bifidobacterium animalis lactis, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 102; (ii) VR2 comprises a nucleic acid sequence with at least 95% sequence identity to SEQ ID NO: 103; (iii) VR3 comprises a nucleic acid sequence with at least 95% sequence identity to SEQ ID NO: 104; (iv) VR4 comprises a nucleic acid sequence with at least 95% sequence identity to SEQ ID NO: 105; (v) VR5 comprises a nucleic acid sequence with at least 95% sequence identity to SEQ ID NO: 106; (vi) VR6 comprises a nucleic acid sequence with at least 95% sequence identity to SEQ ID NO: 107; (vii) VR7 comprises a nucleic acid sequence with at least 95% sequence identity to SEQ ID NO: 108; (viii) VR8 comprises a nucleic acid sequence with at least 95% sequence identity to SEQ ID NO: 109; and (ix) VR9 comprises a nucleic acid sequence with at least 95% sequence identity to SEQ ID NO: 110.

In some embodiments, the Bifidobacterium animalis lactis comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 111; (ii) CR2 comprises a nucleic acid sequence with at least 80% sequence identity to SEQ ID NO: 112; (iii) CR3 comprises a nucleic acid sequence with at least 80% sequence identity to SEQ ID NO: 113; (iv) CR4 comprises a nucleic acid sequence with at least 80% sequence identity to SEQ ID NO: 114; (v) CR5 comprises a nucleic acid sequence with at least 80% sequence identity to SEQ ID NO: 115; (vi) CR6 comprises a nucleic acid sequence with at least 80% sequence identity to SEQ ID NO: 116; (vii) CR7 comprises a nucleic acid sequence with at least 80% sequence identity to SEQ ID NO: 117; (viii) CR8 comprises a nucleic acid sequence with at least 80% sequence identity to SEQ ID NO: 118; (ix) CR9 comprises a nucleic acid sequence with at least 80% sequence identity to SEQ ID NO: 119; and (x) CR10 comprises a nucleic acid sequence with at least 80% sequence identity to SEQ ID NO: 120.

In some embodiments, the present disclosure provides an isolated and purified Bifidobacterium animalis lactis, comprising: a 16S rRNA gene comprising a nucleic acid sequence with at least 97% sequence identity to SEQ ID NO: 101.

In some embodiments, the present disclosure provides an isolated and purified Bifidobacterium animalis lactis having deposit accession number 202103016, 202103017, or 202103019, or a strain having all of the identifying characteristics of Bifidobacterium animalis lactis 202103016, 202103017, or 202103019, or a mutant thereof.

In some embodiments, the present disclosure provides a composition comprising: (a) a purified Lactobacillus plantarum comprising a 16S rRNA nucleic acid sequence that is at least 97% identical to SEQ ID NO: 1; (b) a purified Lactobacillus rhamnosus comprising a 16S rRNA nucleic acid sequence that is at least 97% identical to SEQ ID NO: 21; (c) a purified Lactobacillus acidophilus comprising a 16S rRNA nucleic acid sequence that is at least 97% identical to SEQ ID NO: 41; (d) a purified Lactobacillus paracasei comprising a 16S rRNA nucleic acid sequence that is at least 97% identical to SEQ ID NO: 61 or SEQ ID NO: 181; (e) a purified Bifidobacterium longum comprising a 16S rRNA nucleic acid sequence that is at least 97% identical to SEQ ID NO: 81, SEQ ID NO: 201, or SEQ ID NO: 221; (f) a purified Bifidobacterium animalis lactis comprising a 16S rRNA nucleic acid sequence that is at least 97% identical to SEQ ID NO: 101; (g) a purified Veillonella dispar comprising a 16S rRNA nucleic acid sequence that is at least 97% identical to SEQ ID NO: 121; (h) a purified Veillonella parvula comprising a 16S rRNA nucleic acid sequence that is at least 97% identical to SEQ ID NO: 141; and/or (i) a purified Veillonella atypica comprising a 16S rRNA nucleic acid sequence that is at least 97% identical to SEQ ID NO: 161.

In some embodiments, the present disclosure provides a composition comprising: (a) a purified Lactobacillus plantarum comprising a 16S rRNA nucleic acid sequence that is at least 97% identical to SEQ ID NO: 1; (b) a purified Lactobacillus rhamnosus comprising a 16S rRNA nucleic acid sequence that is at least 97% identical to SEQ ID NO: 21; and/or (c) a purified Lactobacillus acidophilus comprising a 16S rRNA nucleic acid sequence that is at least 97% identical to SEQ ID NO: 41.

In some embodiments, the present disclosure provides a composition comprising: (a) a purified Lactobacillus plantarum comprising a 16S rRNA nucleic acid sequence that is at least 97% identical to SEQ ID NO: 1; (b) a purified Lactobacillus rhamnosus comprising a 16S rRNA nucleic acid sequence that is at least 97% identical to SEQ ID NO: 21; (c) a purified Lactobacillus acidophilus comprising a 16S rRNA nucleic acid sequence that is at least 97% identical to SEQ ID NO: 41; (d) a purified Bifidobacterium longum comprising a 16S rRNA nucleic acid sequence that is at least 97% identical to SEQ ID NO: 81, SEQ ID NO: 201, or SEQ ID NO: 221; and/or (e) a purified Bifidobacterium animalis lactis comprising a 16S rRNA nucleic acid sequence that is at least 97% identical to SEQ ID NO: 101.

In some embodiments, the present disclosure provides a composition comprising: (a) a purified Lactobacillus plantarum having the deposit accession number 202103009; (b) a purified Lactobacillus rhamnosus having the deposit accession number 202103010; (c) a purified Lactobacillus acidophilus having the deposit accession number 202103008; (d) a purified Lactobacillus paracasei having the deposit accession number 202103012 or 202103018; (e) a purified Bifidobacterium longum having the deposit accession number 202103013, 202103014, or 202103015; (f) a purified Bifidobacterium animalis lactis having the deposit accession number 202103016, 202103017, or 202103019; (g) a purified Veillonella dispar having the deposit accession number PTA-126861; (h) a purified Veillonella parvula having the deposit accession number PTA-126859; and/or (i) a purified Veillonella atypica having the deposit accession number PTA-126860.

In some embodiments, the composition is administered at a dose that alters the microbiome, enhances exercise endurance, improves athletic performance, increases muscle mass, increases muscular strength, prevents the loss of muscle mass, enhances physical recovery, improves digestion, improves metabolism, increases immunity, reduces inflammation, and/or improves sleep quality in a subject in need thereof.

In some embodiments, the composition is a food composition, a beverage composition or a dietary supplement composition.

In some embodiments, the present disclosure provides a method of altering the microbiome of a subject comprising administering an effective dose of any of the compositions disclosed herein to the subject.

In some embodiments, the present disclosure provides a method of reducing the level of lactic acid and/or lactates in blood of a subject comprising administering an effective dose of any of the compositions disclosed herein to the subject.

In some embodiments, the present disclosure provides a method of increasing the level of propionic acid and/or propionates in blood of a subject comprising administering an effective dose of any of the compositions disclosed herein to the subject.

In some embodiments, the present disclosure provides a method of increasing the level of acetic acid and/or acetates in blood of a subject comprising administering an effective dose of any of the compositions disclosed herein to the subject.

In some embodiments, the present disclosure provides a method of enhancing exercise endurance of a subject comprising administering an effective dose of any of the compositions disclosed herein to the subject.

In some embodiments, the present disclosure provides a method of improving athletic performance of a subject in need thereof comprising administering an effective dose of any of the compositions disclosed herein to the subject.

In some embodiments, the present disclosure provides a method of enhancing recovery from physical exercise in a subject comprising administering an effective dose of any of the compositions disclosed herein to the subject.

In some embodiments, the present disclosure provides a method of reducing inflammation in a subject in need thereof comprising administering an effective dose of any of the compositions disclosed herein to the subject.

In some embodiments, the present disclosure provides a method of increasing muscle mass and/or muscular strength of a subject in need thereof comprising administering an effective dose of any of the compositions disclosed herein to the subject.

In some embodiments, the present disclosure provides a method of preventing the loss of muscle mass of a subject in need thereof comprising administering an effective dose of any of the compositions disclosed herein to the subject.

In some embodiments, the present disclosure provides a method of improving sleepy quality in a subject in need thereof comprising administering an effective dose of any of the compositions disclosed herein to the subject.

In some embodiments, the administration is via oral, enteric, gastrointestinal, or rectal route.

In some embodiments, the subject is a human subject.

In some embodiments, the dose is in the range of about 10⁴ CFU to about 10¹⁶ CFU. In some embodiments, the dose is in the range of about 10⁹ CFU to about 10¹¹ CFU. In some embodiments, the dose is in the range of about 5×10⁹ CFU to about 10¹⁰ CFU. In some embodiments, the dose is about 3.5×10¹⁰ CFU.

### Budapest Treaty on the International Recognition of the Deposit of Microorganisms for the Purpose of Patent Procedures

Microorganisms described in this application were deposited with the Bigelow National Center for Marine Algae and Microbiota, located at 60 Bigelow Drive, East Boothbay, Me. 04544, USA. Microorganisms described in this application were also deposited with the American Type Culture Collection (ATCC®), located at 10801 University Blvd., Manassas, Va. 20110, USA and/or the United States Department of Agriculture (USDA) Agricultural Research Service (ARS) Culture Collection (NRRL®), located at 1815 N. University St., Peoria, Ill. 61604, USA. The deposits were made under the terms of the Budapest Treaty on the International Recognition of the Deposit of Microorganisms for the Purposes of Patent Procedure. The predicted taxonomy, 16S rRNA SEQ ID NOs, deposit accession numbers, and corresponding dates of deposit for the microorganisms described in this application are provided in Table 1 below. The compositions of the disclosure may contain any one, or any combination, of the microbes listed below in Table 1. The microbes of Table 1 may be administered as a single microbial product and/or as a microbial consortia comprising a combination of microbes from Table 1. The products and methods of the disclosure may use any one of, or any combination of, microbes as listed below in Table 1.

TABLE 1

| Predicted Taxa of | | 16S rRNA | Depository, Deposit No. |
|---|---|---|---|
| Isolated Microbes | Strain ID | SEQ ID NO | (Deposit Date) |
| Lactobacillus | FB00015 | 1 | Bigelow, 202103009 |
| plantarum | | | (3 Mar. 2021) |
| Lactobacillus | FB00047 | 21 | Bigelow, 202103010 |
| rhamnosus | | | (3 Mar. 2021) |
| Lactobacillus | FB00012 | 41 | Bigelow, 202103008 |
| acidophilus | | | (3 Mar. 2021) |
| Lactobacillus | FB00014 | 61 | Bigelow, 202103012 |
| paracasei | | | (12 Mar. 2021) |
| Bifidobacterium | FB00032 | 81 | Bigelow, 202103014 |
| longum | | | (12 Mar. 2021) |
| Bifidobacterium | FB00034 | 101 | Bigelow, 202103016 |
| animalis lactis | | | (12 Mar. 2021) |
| Veillonella | FB55 | 121 | ATCC, PTA-126861 |
| dispar | | | (15 Oct. 2020) |
| Veillonella | FB53 | 141 | ATCC, PTA-126859 |
| parvula | | | (15 Oct. 2020) |
| Veillonella | FB54 | 161 | ATCC, PTA-126860 |
| atypica | | | (15 Oct. 2020) |
| Lactobacillus | FB00046 | 181 | Bigelow, 202103018 |
| paracasei | | | (12 Mar. 2021) |
| Bifidobacterium | FB00016 | 201 | Bigelow, 202103013 |
| longum | | | (12 Mar. 2021) |
| Bifidobacterium | FB00033 | 221 | Bigelow, 202103015 |
| longum | | | (12 Mar. 2021) |
| Bifidobacterium | FB00044 | | Bigelow, 202103017 |
| animalis lactis | | | (12 Mar. 2021) |
| Bifidobacterium | FB00048 | | Bigelow, 202103019 |
| animalis lactis | | | (12 Mar. 2021) |

### BRIEF DESCRIPTION OF THE DRAWINGS

FIG. 1A shows the sequence of 16S rRNA gene identified in the Lactobacillus plantarum strain (SEQ ID NO: 1), and the variable and constant regions within the gene (FIG. 1A); the sequence of 16S rRNA gene identified in the Lactobacillus rhamnosus strain (SEQ ID NO: 21), and the variable and constant regions within the gene (FIG. 1B); the sequence of 16S rRNA gene identified in the Lactobacillus acidophilus strain (SEQ ID NO: 41), and the variable and constant regions within the gene (FIG. 1C); the sequence of 16S rRNA gene identified in the Lactobacillus paracasei strain (SEQ ID NO: 61), and the variable and constant regions within the gene (FIG. 1D); and the sequence of 16S rRNA gene identified in the Lactobacillus paracasei strain (SEQ ID NO: 181), and the variable and constant regions within the gene (FIG. 1E). The variable regions (VR1 through VR9) are indicated by sequences that are italicized and underlined, while the constant regions (CR1 through CR10) are indicated by sequences that are in bold. The regions of the 16S rRNA genes are ordered in the following manner: 5′-CR1-VR1-CR2-VR2-CR3-VR3-CR4-VR4-CR5-VR5-CR6-VR6-CR7-VR7-CR8-VR8-CR9-VR9-CR10-3′

FIG. 2A shows the the sequence of 16S rRNA gene identified in the Bifidobacterium longum strain (SEQ ID NO: 81), and the variable and constant regions within the gene (FIG. 2A); the sequence of 16S rRNA gene identified in the Bifidobacterium animalis lactis strain (SEQ ID NO: 101), and the variable and constant regions within the gene (FIG. 2B); the sequence of 16S rRNA gene identified in the Bifidobacterium longum strain (SEQ ID NO: 201), and the variable and constant regions within the gene (FIG. 2C); and the sequence of 16S rRNA gene identified in the Bifidobacterium longum strain (SEQ ID NO: 221), and the variable and constant regions within the gene (FIG. 2D). The variable regions (VR1 through VR9) are indicated by sequences that are italicized and underlined, while the constant regions (CR1 through CR10) are indicated by sequences that are in bold. The regions of the 16S rRNA genes are ordered in the following manner: 5′-CR1-VR1-CR2-VR2-CR3-VR3-CR4-VR4-CR5-VR5-CR6-VR6-CR7-VR7-CR8-VR8-CR9-VR9-CR10-3′

FIG. 3A shows the sequence of 16S rRNA gene identified in the Veillonella dispar strain (SEQ ID NO: 121), and the variable and constant regions within the gene; FIG. 3B shows the sequence of 16S rRNA gene identified in the Veillonella parvula strain (SEQ ID NO: 141), and the variable and constant regions within the gene; and FIG. 3C shows the sequence of 16S rRNA gene identified in the Veillonella atypica strain (SEQ ID NO: 161), and the variable and constant regions within the gene. The variable regions (VR1 through VR9) are indicated by sequences that are italicized and underlined, while the constant regions (CR1 through CR10) are indicated by sequences that are in bold. The regions of the 16S rRNA genes are ordered in the following manner: 5′-CR1-VR1-CR2-VR2-CR3-VR3-CR4-VR4-CR5-VR5-CR6-VR6-CR7-VR7-CR8-VR8-CR9-VR9-CR10-3′.

FIG. 4 shows the concentration of lactate, acetate and propionate produced by Veillonella atypica having deposit accession number PTA-126860, and compositions comprising this Veillonella atypica strain and one or more lactate producing bacteria, as measured by Mass Spectrometry. “MRS” refers to the commercial medium that is typically used to grow the Lactobacillus and Bifidobacterium strains. “MRS lactate” refers to MRS medium supplemented with sodium lactate. L=lactate; A=acetate; P=propionate.

FIG. 5A provides a summary of the subject demographics. FIG. 5B shows the dietary preferences, nutritional supplements, and desired benefits and ingredients most important to the subjects.

FIG. 6 shows the impact of low and high dose probiotic supplementation on digestion.

FIG. 7 shows the impact of probiotic supplementation on sleep quality, fatigue, and energy.

FIG. 8A shows exercise intensity of the subjects at baseline, one week, and two weeks following probiotic supplementation. FIG. 8B shows soreness after exercise of subjects at baseline, one week, and two weeks following low or high dose probiotic supplementation. FIG. 8C shows the feeling during exercise of subjects at baseline, one week, and two weeks following low or high dose probiotic supplementation. FIG. 8D shows the recovery time after exercise of subjects at baseline, one week, and two weeks following low or high dose probiotic supplementation.

FIG. 9A provides an overall summary of the results from the human performance testing of probiotic supplementation. FIG. 9B shows the preferred format and frequency of use of the probiotic supplement for subjects.

FIG. 10A provides a summary of the subject demographics. FIG. 10B shows exercise frequency, exercise regimen, and purpose of training and exercise of the subjects. FIG. 10C shows the dietary preferences, nutritional supplements, and desired benefits and ingredients most important to the subjects.

FIG. 11A shows the impact of probiotic supplementation on current health and wellbeing overtime. FIG. 11B shows the impact of probiotic supplementation on general health, digestion, and fitness compared to personal normal.

FIG. 12 shows the impact of probiotic supplementation on the change in bowel movement quality over time.

FIG. 13A shows the impact of probiotic supplementation on sleep quality overtime. FIG. 13B shows the impact of probiotic supplementation on fatigue frequency overtime. FIG. 13C shows the impact of probiotic supplementation on energy level overtime. FIG. 13D shows the impact of probiotic supplementation on stress level overtime.

FIG. 14A shows the feeling during exercise of subjects at baseline, one week, and two weeks following probiotic supplementation. FIG. 14B shows the feeling after exercise of subjects at baseline, one week, and two weeks following probiotic supplementation. FIG. 14C shows the recovery time after exercise of subjects at baseline, one week, and two weeks following probiotic supplementation.

FIG. 15A shows the improvement in current health and wellness in subjects following one or two weeks of probiotic supplementation. NaN, not a number (unrepresentable). FIG. 15B shows the improvement in general health, digestion, and fitness in subjects following two weeks of probiotic supplementation. NaN, not a number (unrepresentable). FIG. 15C shows the improvement in sleep quality in subjects following one or two weeks of probiotic supplementation. NaN, not a number (unrepresentable). FIG. 15D shows the improvement in fatigue frequency in subjects following two weeks of probiotic supplementation. NaN, not a number (unrepresentable). FIG. 15E shows the improvement in fatigue frequency in subjects following two weeks of probiotic supplementation. FIG. 15F shows the improvement in normalized fatigue frequency in subjects following two weeks of probiotic supplementation. FIG. 15G shows the improvement in feeling during a workout in subjects following one or two weeks of probiotic supplementation. NaN, not a number (unrepresentable). FIG. 1511 shows the improvement in soreness in subjects following one or two weeks of probiotic supplementation. NaN, not a number (unrepresentable). FIG. 15I shows the improvement in recovery time after a workout in subjects following one or two weeks of probiotic supplementation. NaN, not a number (unrepresentable). FIG. 15J shows the improvement in bowel movements in subjects following two weeks of probiotic supplementation. NaN, not a number (unrepresentable). FIG. 15K shows the improvement in energy in subjects following two weeks of probiotic supplementation. NaN, not a number (unrepresentable). FIG. 15L shows the improvement in stress in subjects following two weeks of probiotic supplementation. NaN, not a number (unrepresentable).

FIG. 16A shows the overall experience of subjects in the study that received low or high dose probiotic supplementation. Overall experience was measured on a scale of 1 to 5 with 1 as very poor and 5 as excellent. Most subjects reported their overall experience as a 4 or 5 with no significant difference between low and high dose probiotic supplementation. FIG. 16B shows the changes in bowel movements following probiotic supplementation in subjects with high, medium, or low overall experience. FIG. 16C shows the energy level at week 2 of probiotic supplementation in subjects with high, medium, or low overall experience. FIG. 16D shows the changes in general health, digestion, and fitness following probiotic supplementation in subjects with high, medium, or low overall experience. FIG. 16E shows heart rate variability (HRV), resting heart rate (RHR), respiratory, recovery, and sleep scores in subjects receiving probiotic supplementation overtime. Results were measured using the WHOOP fitness tracking devise.

FIG. 17 shows the preferred format and frequency of use of the probiotic supplement for subjects.

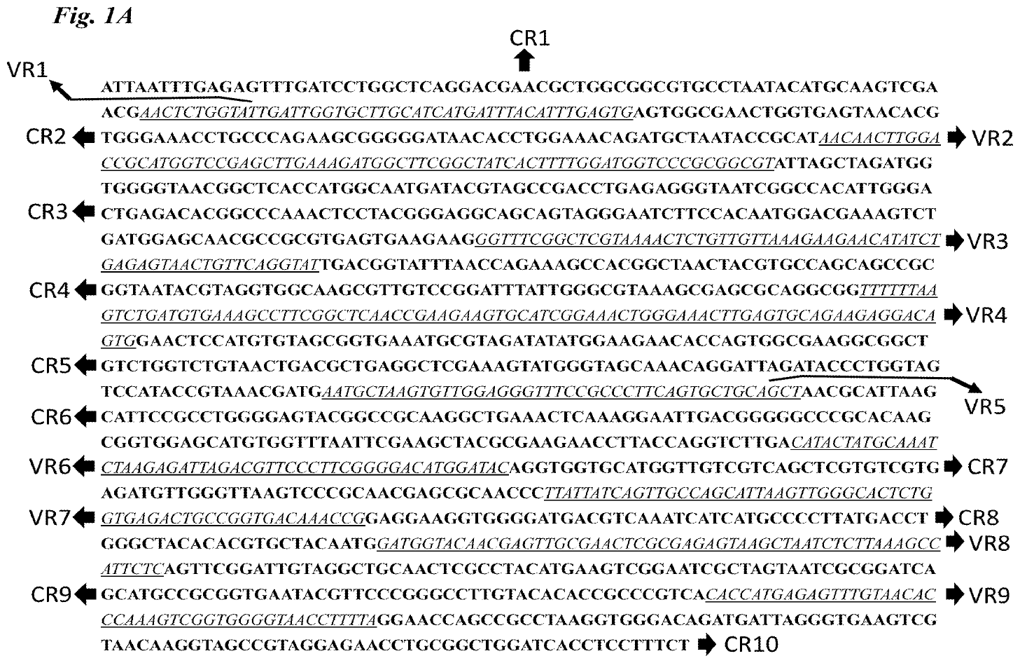

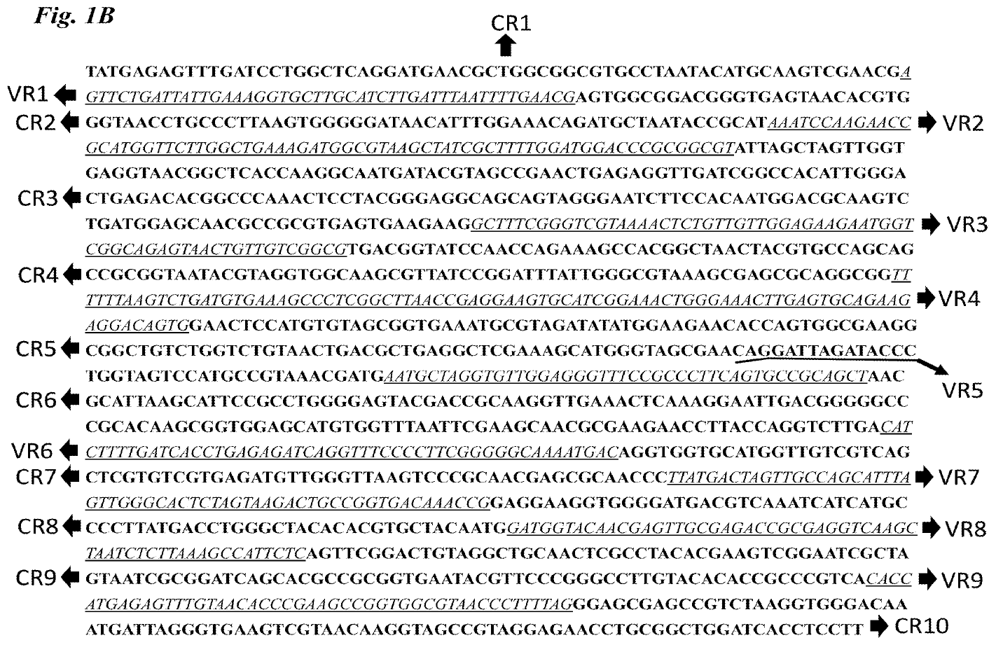

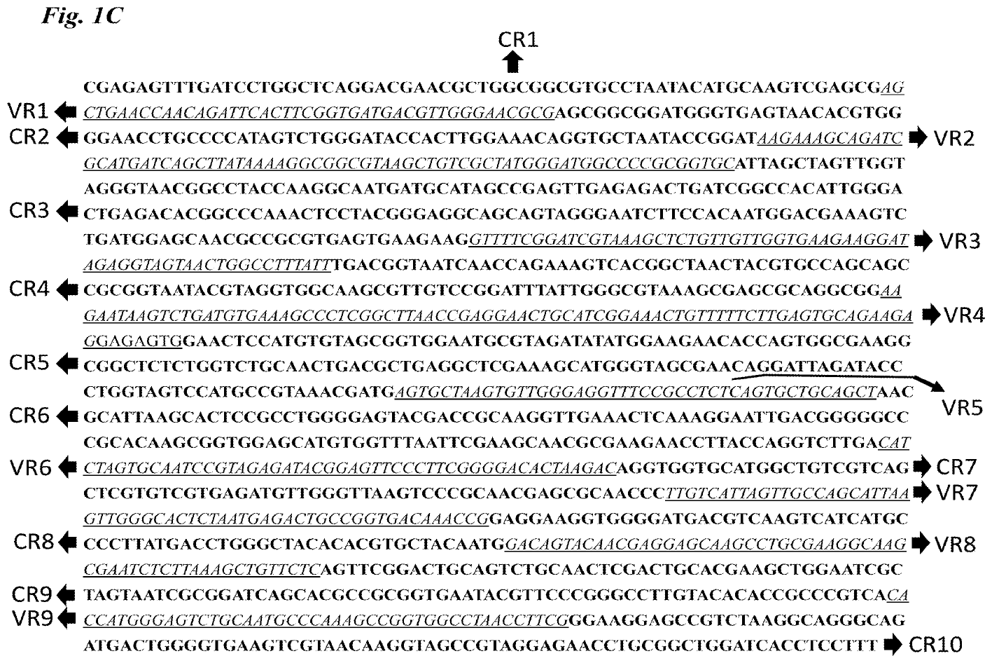

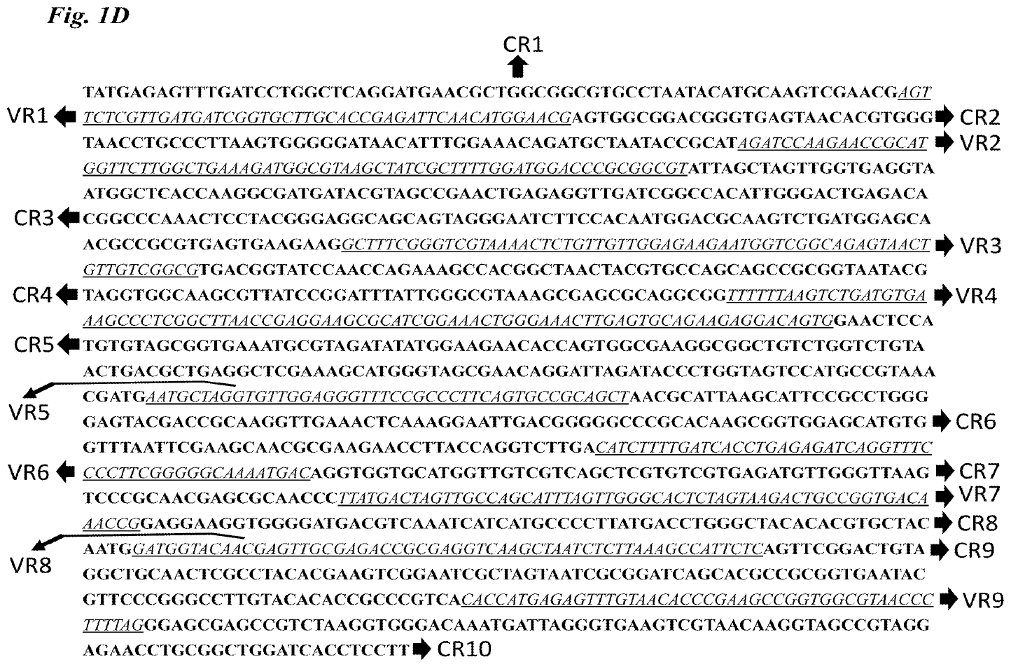

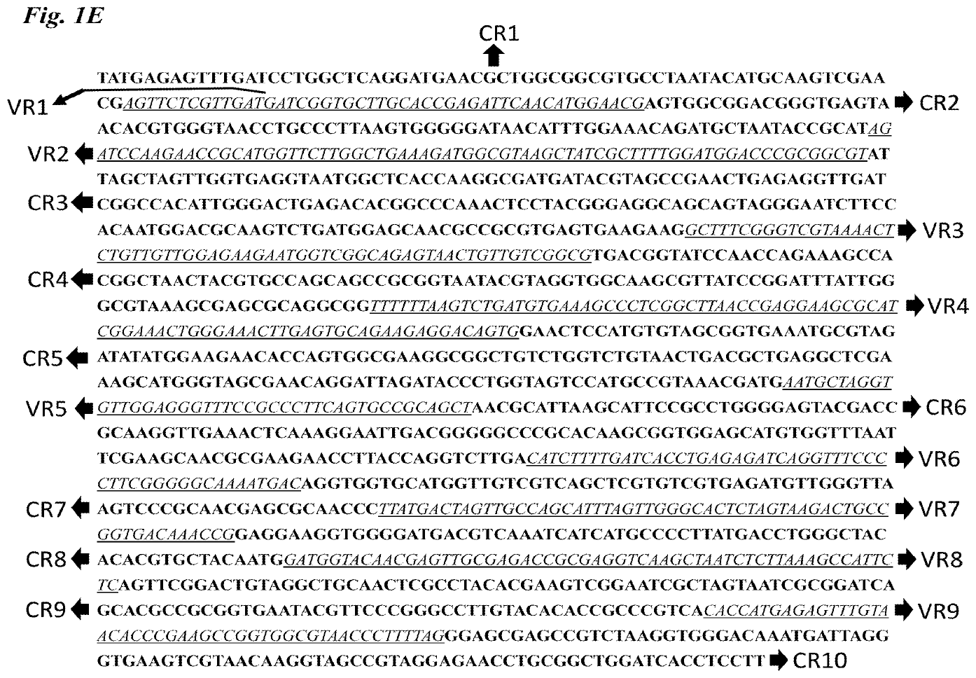

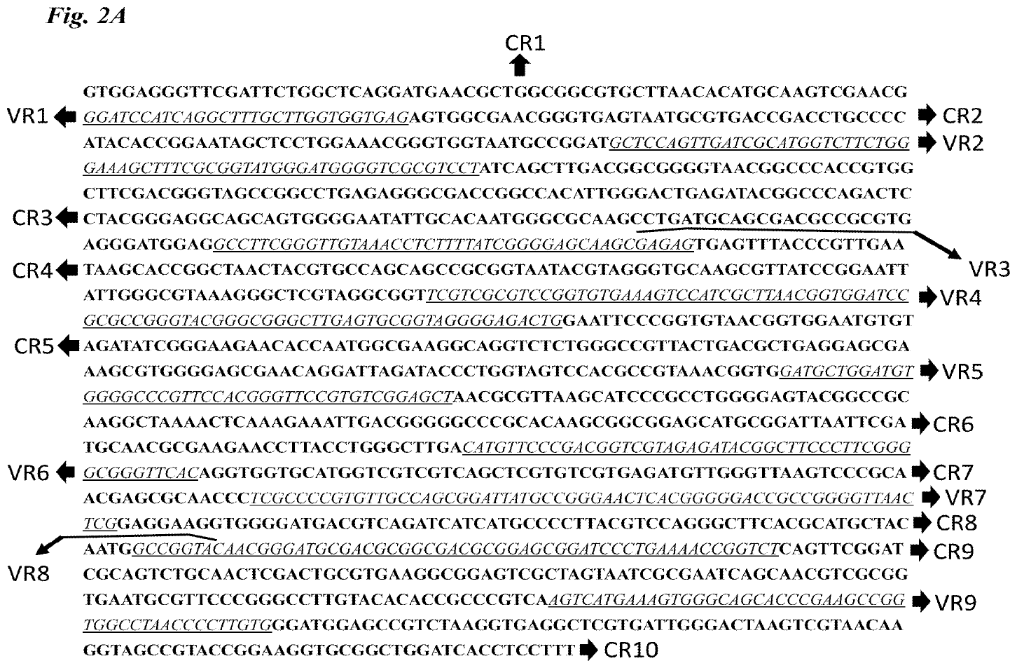

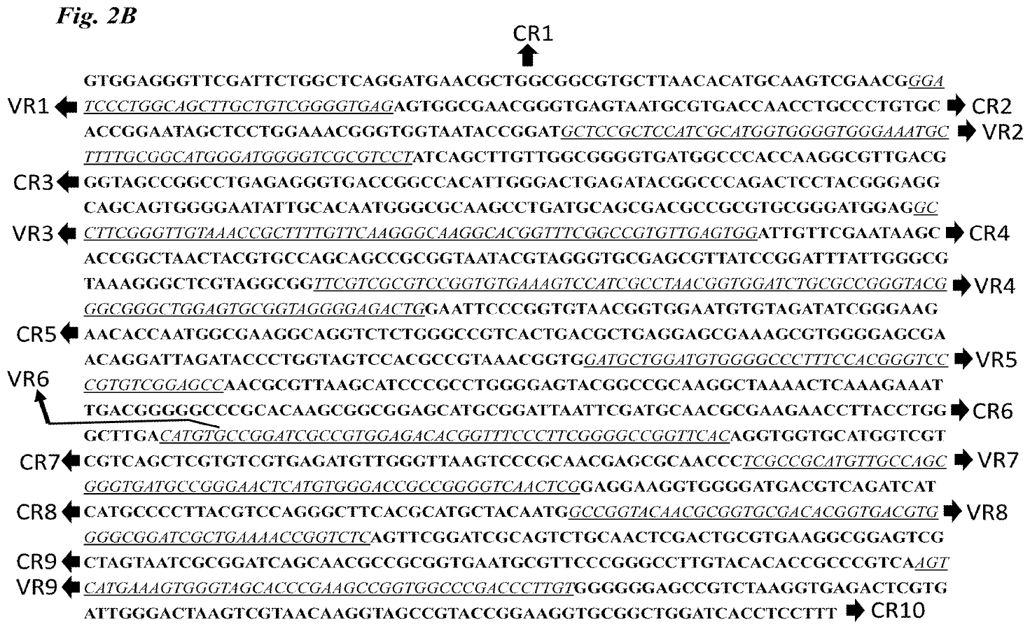

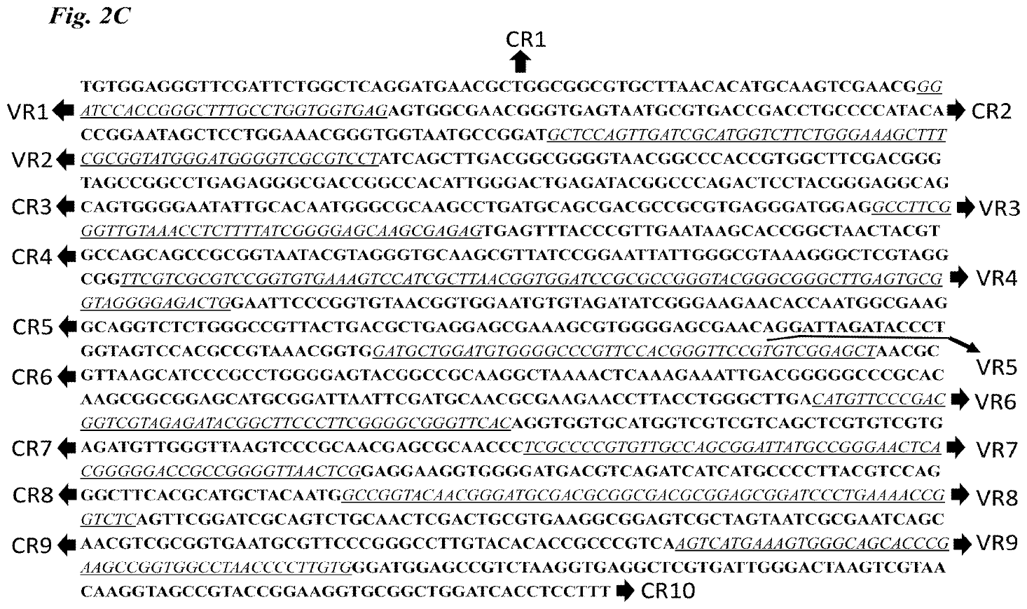

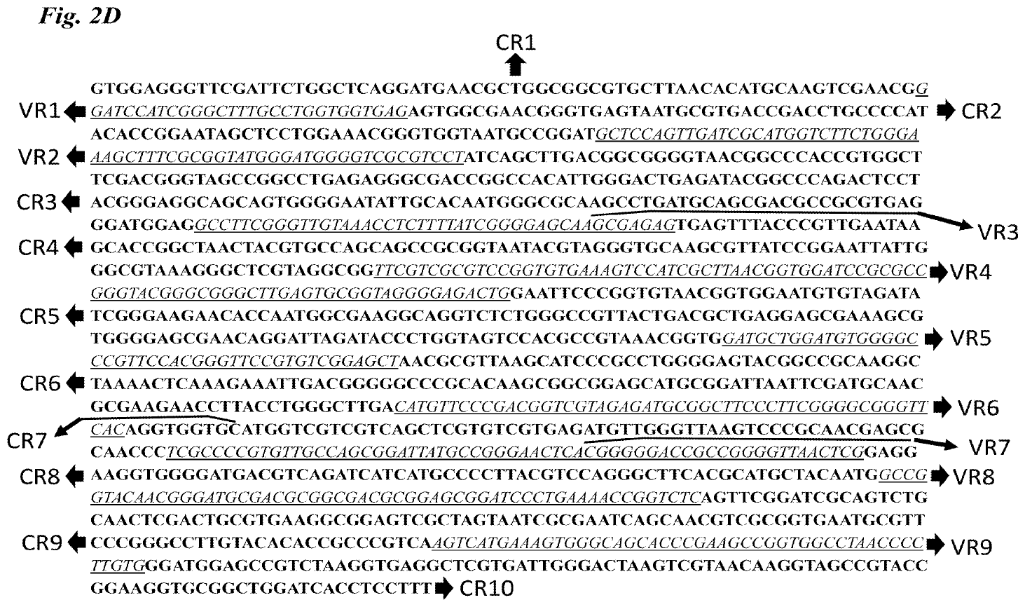

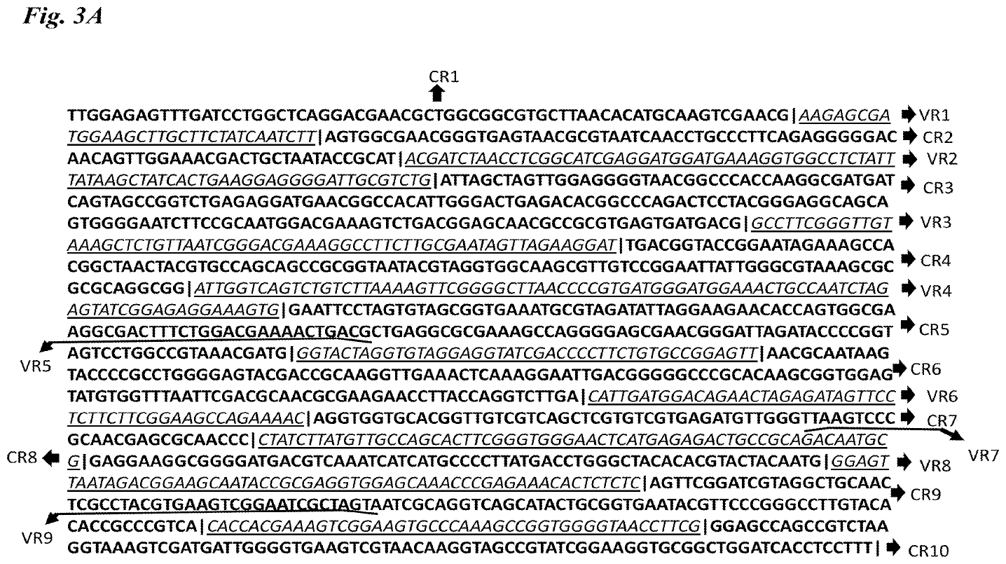

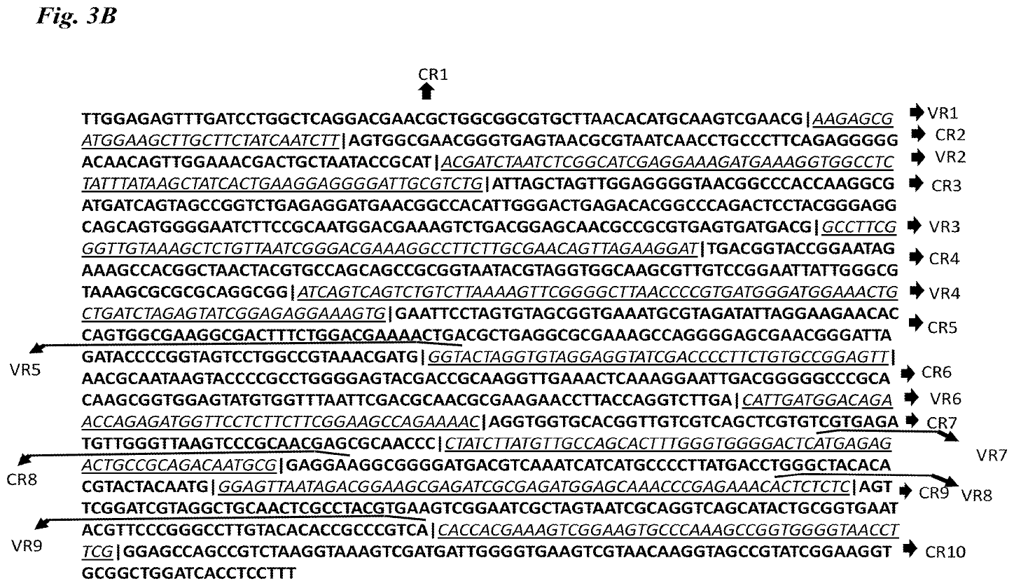

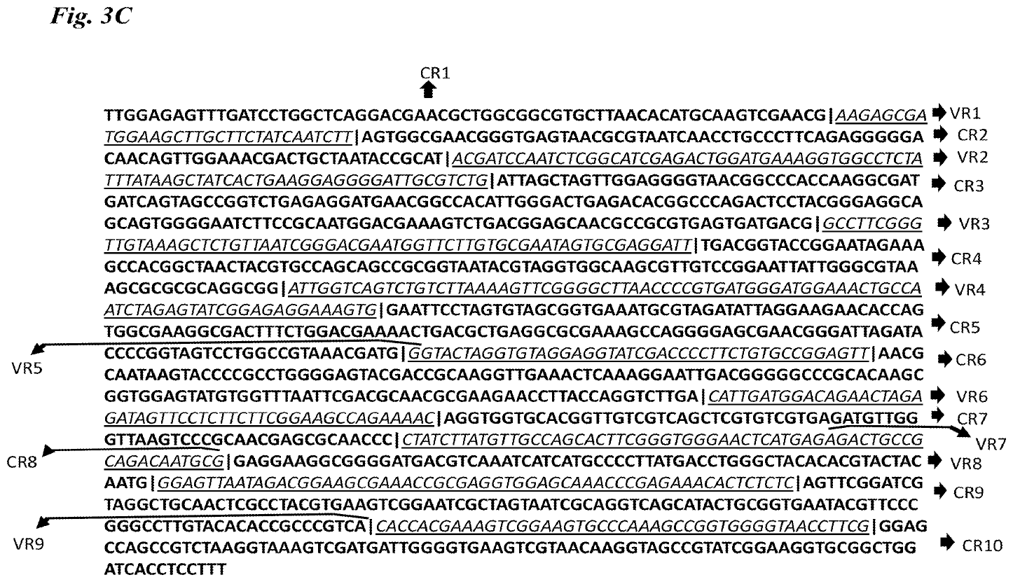

### DETAILED DESCRIPTION

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

As used herein, terms such as “a,” “an,” and “the” include singular and plural referents unless the context clearly demands otherwise.

As used herein, the term “about” when preceding a numerical value indicates the value plus or minus a range of 10%. For example, “about 100” encompasses 90 and 110.

As used herein, the term “subject” includes humans and other animals. Typically, the subject is a human. For example, the subject may be an adult or a teenager. In some embodiments, the adults are seniors about 65 years or older, or about 60 years or older. In some embodiments, the subject may be an animal which is being trained or is skilled in athletic activities, such as a horse. In some embodiments, the subject is a human athlete, or a person training to be an athlete.

As used herein, the term “athlete” refers to a human subject who follows a regular exercise regimen. The term “regular exercise regimen” is not limited, and may be determined by a person of ordinary skill in the art, a physician or an athletic trainer. For instance, a regular exercise regimen may comprise performing a “physical exercise” at least once a week, for example twice a week, or thrice a week. As used herein, the term “non-athlete” refers to a human subject who does not follow a “regular exercise regimen”. In some embodiments, the athlete is a person trained and/or skilled in any form of physical exercise. In some embodiments, the athlete possesses enhanced physical strength, agility, endurance, speed and/or stamina as compared to the non-athlete. Methods to measure physical strength, agility, endurance, speed and/or stamina are commonly known, and may be chosen by one of ordinary skill in the art.

As used herein, the term “physical exercise” refers to any activity that involves physical exertion. In some embodiments, physical exercise enhances or maintains physical fitness, strengthens muscles and the cardiovascular system, promotes weight loss or maintenance, and/or enhances physical and/or mental health. Non-limiting examples of physical exercise include running, cycling, swimming, brisk walking, skipping rope, rowing, hiking, dancing, playing tennis, continuous training, long distance running, push-ups, pull-ups, lunges, squats, bench press, weight training, functional training, eccentric training, interval training, sprinting, and high-intensity interval training.

As used herein, “athletic performance” or “exercise performance” refers to one or more objective factors related to a particular physical exercise for a subject. In some embodiments, the objective factor is measurable and defined, such as the distance of a run, the height of a jump, or the distance an object is thrown. An improvement of athletic performance comprises an improvement of one or more objective factors related to the particular physical exercise for the particular subject. For instance, for a race, an improvement of athletic performance may comprise an increase in the distance run, a decrease in the time taken to run a particular distance, or a combination thereof. In some embodiments, the objective factor is exercise endurance.

As used herein, “exercise endurance” (interchangeably referred to herein as “training endurance” and “performance endurance”) refers to an ability to perform a particular physical exercise over a given period of time, and/or perform a physical exercise of a particular strenuousness. In some embodiments, increasing exercise endurance comprises an increase in the time that a physical exercise is performed, and/or an increase in the strenuousness of the exercise until exhaustion time. As used herein, “exhaustion time” or “exhaustion point” refers to the endpoint of a period of time, or a particular strenuousness, beyond which a particular physical exercise cannot be performed by a subject, due to, for example, fatigue.

As used herein, “microbiome” refers to the collection of microorganisms that inhabit the digestive tract or gastrointestinal (GI) tract of an animal (including the GI tract of a human) and the microorganism's physical environment (i.e. the microbiome has a biotic and abiotic component). The microbiome is fluid and may be modulated by numerous naturally occurring and artificial conditions (e.g., change in diet, disease, antimicrobial agents, influx of additional microorganisms, etc.). The modulation or optimization or alteration or shifting of the gut microbiome achieved via administration of the microbial strains and/or compositions of the present disclosure, can take the form of: (a) changing the diversity; i.e., increasing or decreasing a particular Family, Genus, Species, strain or functional grouping of microbes (i.e. alteration of the biotic component of the microbiome); (b) increasing or decreasing ratios of a particular Family, Genus, Species, strain or functional grouping of microbes; (c) increasing or decreasing polyphenols, proteins and/or metabolic compounds such as lactic acid, lactates, propionic acid, propionates, acetic acid, or acetates (i.e. alteration of the abiotic components of the microbiome) and/or (d) changing the quality of the microbiome; such as, for example increasing the proportion of microbes associated with enhanced exercise endurance.

The terms “microorganism” or “microbe” in certain instances may refer to an organism of microscopic size, to a single-celled organism, and/or to any virus particle. The definition of microorganism used herein includes Bacteria, Archaea, single-celled Eukaryotes (protozoa, fungi, and ciliates), and viral agents.

As used herein, “athlete-associated gut microbe” refers to a Family, Genus, Species, strain or functional grouping of a microbe that inhabits the digestive tract or gastrointestinal (GI) tract of an athlete.

As used herein, an “effective dose” or “effective amount” refers to an amount of substance able to achieve a desired outcome; for example, an amount of any one of the microbial strains or compositions disclosed herein that is sufficient to affect a desired outcome, such as an improvement of athletic performance.

As used herein, “inflammation” refers to a complex biological response of body tissues to harmful stimuli, such as pathogens, damaged cells, or irritants, and is a protective response involving immune cells, blood vessels, and molecular mediators, giving rise to one or more signs of heat, pain, redness, swelling, and loss of function. In some embodiments, the inflammation may be associated with, promoted by, or caused by physical exercise.

As used herein, “inflammatory cytokine” refers to a signaling molecule produced by cells (e.g., an immune cell) that promotes inflammation in a subject. Examples of inflammatory cytokines include, but are not limited to, IL-1β, IL-4, IL-6, IL-8, TNF-α, IL-12p′70, IL-17, and IFN-γ.

As used herein, “recovery from physical exercise” refers to the process by which one or more parts of the body, such as muscles, recover from the effect of physical exercise. In some embodiments, “recovery from physical exercise” comprises an alleviation of inflammation associated with physical exercise. The recovery from physical exercise may be associated with or promoted by one or more of the following: rest, sleep, hydration, stretching, nutrition, and massage. In some embodiments, recovery from physical exercise comprises a decrease in the levels of lactic acid and/or lactates in the blood, and/or an increase in the levels of any one or more of the following: propionic acid, propionates, acetic acid, and acetates, in the blood. In some embodiments, “enhancing recovery from physical exercise” comprises a decrease in the time taken to recover from physical exercise.

An “improvement in digestion” as used herein refers to an improvement in fecal consistency, decreased incidence of diarrhea, reduced constipation, increased incidence of bowel movements, decreased incidence of bowel movements, and/or reduced abdominal symptoms such as gas, bloating, and abdominal pain.

An “improvement in metabolism” as used herein refers to increased metabolism of fat in a subject or a cell, tissue, or organ thereof.

As used herein, “sleep quality” refers to how well a subject sleeps and includes aspects such as sleep initiation, sleep maintenance, sleep quantity, and refreshment upon awakening.

As used herein, short chain fatty acids (SCFAs) refer to fatty acids with fewer than 6 carbon atoms. In some embodiments, SCFAs are generated in the gastrointestinal tract of a subject, for example, from the fermentation of indigestible foods by the gut microbiome. Non-limiting examples of SCFAs are formic acid, acetic acid, propionic acid, butyric acid, isobutyric acid, valeric acid, isovaleric acid, and 2-methylbutanoic acid.

### Athlete-Associated Gut Microbes, and Compositions Comprising the Same

The disclosure provides microbes that inhabit the digestive tract or gastrointestinal (GI) tract of an athlete, referred to herein as athlete-associated gut microbes. In some embodiments, the athlete-associated gut microbes inhabit the ileum or colon of the athlete. In some embodiments, the athlete-associated gut microbes are capable of metabolizing lactic acid and/or lactates. In some embodiments, the athlete-associated gut microbes are capable of metabolizing lactic acid and/or lactates into products, such as, for example, short-chain fatty acids (SCFAs), such as acetate and propionate. Therefore, in some embodiments, the one or more athlete-associated gut microbes is capable of producing propionic acid, propionates, acetic acid and/or acetates. In some embodiments, the athlete-associated gut microbes comprise one or more enzymes required for the conversion of lactate into SCFAs, such as acetate and propionate. In some embodiments, the athlete-associated gut microbes comprise at least one gene encoding at least one enzyme of the methylmalonyl-CoA pathway. In some embodiments, the athlete-associated gut microbes comprise one or more genes encoding all the enzymes of the methylmalonyl-CoA pathway. In some embodiments, the athlete-associated gut microbes comprise succinate-CoA transferase. Without being bound by a theory, it is thought that succinate-CoA transferase contributes to the conversion of lactate into SCFAs and propionate.

In some embodiments, the athlete-associated gut microbes are recombinant microbes, which have been engineered to metabolize lactic acid and/or lactates into products, such as, for example, short-chain fatty acids (SCFAs), such as acetate and propionate. In some embodiments, the recombinant athlete-associated gut microbes are engineered to encode one or more enzymes of the methylmalonyl-CoA pathway.

In some embodiments, the athlete-associated gut microbes are associated with, promote or cause enhanced exercise endurance, improved athletic performance, reduced inflammation, enhanced recovery from physical exercise, or any combination thereof in the athlete. In some embodiments, the population (or number or abundance) of one or more athlete-associated gut microbe is different (for example, higher or lower) in an athlete, as compared to a non-athlete. In some embodiments, the proportion of one or more athlete-associated gut microbe among the total number of gut microbes is different (higher or lower) in an athlete, as compared to a non-athlete.

In some embodiments, the activity of one or more athlete-associated gut microbes to metabolize lactic acid and/or lactates is higher in the athlete, as compared to a non-athlete. In some embodiments, the activity of one or more athlete-associated gut microbes to produce propionic acid, propionates, acetic acid and/or acetates is higher, as compared to a non-athlete. In some embodiments, any one or more of the microbial strains disclosed herein are resistant to gastric and bile acids. In some embodiments, any one or more of the microbial strains disclosed herein are capable of competing with pathogens for adhesion sites in the gut tissue. In some embodiments, any one or more of the microbial strains disclosed herein are capable of producing pathogen inhibitory substances, e.g. bacteriocidins and organic acids.

In some embodiments, the athlete-associated gut microbe comprises a nucleic acid sequence comprising at least about 70% sequence identity to any one of SEQ ID NOs: 1-240, for example, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to any one of SEQ ID NOs: 1-240, including all subranges and values that lie therebetween.

### (a) Athlete-Associated Lactobacillus Strains

In some embodiments, the athlete-associated gut microbe comprises a microbial strain belonging to the genus Lactobacillus. In some embodiments, the microbial strain belonging to the genus Lactobacillus is an isolated and purified strain. In some embodiments, the microbial strain belonging to the genus Lactobacillus comprises a 16S ribosomal RNA (rRNA) gene comprising a nucleic acid sequence comprising at least one variable region. In some embodiments, the microbial strain belonging to the genus Lactobacillus comprises a 16S rRNA gene comprising a nucleic acid sequence comprising at least one constant region. In some embodiments, the at least one variable region (VR) is selected from the group consisting of: VR1, VR2, VR3, VR4, VR5, VR6, VR7, VR8, VR9, and any combination thereof. In some embodiments, the at least one constant region (CR) selected from the group consisting of: CR1, CR2, CR3, CR4, CR5, CR6, CR7, CR8, CR9, CR10, and any combination thereof.

In some embodiments, the microbial strain belonging to the genus Lactobacillus comprises a 16S rRNA gene comprising a nucleic acid sequence comprising the following variable regions: VR1, VR2, VR3, VR4, VR5, VR6, VR7, VR8, and VR9, and the following constant regions: CR1, CR2, CR3, CR4, CR5, CR6, CR7, CR8, CR9, CR10. In some embodiments, the VRs and CRs are ordered as shown below: 5′-CR1-VR1-CR2-VR2-CR3-VR3-CR4-VR4-CR5-VR5-CR6-VR6-CR7-VR7-CR8-VR8-CR9-VR9-CR10-3′

(i) Lactobacillus plantarum Strains

In some embodiments, the athlete-associated gut microbe comprises a strain of Lactobacillus plantarum. In some embodiments, the VR1 of Lactobacillus plantarum comprises a nucleic acid sequence with at least about 70% sequence identity to SEQ ID NO: 2, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 2, including all subranges and values that lie therebetween.

In some embodiments, the VR2 of Lactobacillus plantarum comprises a nucleic acid sequence with at least about 70% sequence identity to SEQ ID NO: 3, for example, at least about 70%, at least about 75%, at least about 80%, at least about 85%, at least about about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 3, including all subranges and values that lie therebetween.

In some embodiments, the VR3 of Lactobacillus plantarum comprises a nucleic acid sequence with at least about 70% sequence identity to SEQ ID NO: 4, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 4, including all subranges and values that lie therebetween.

In some embodiments, the VR4 of Lactobacillus plantarum comprises a nucleic acid sequence with at least about 70% sequence identity to SEQ ID NO: 5, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about ID NO: 5, including all subranges and values that lie therebetween.

In some embodiments, the VR5 of Lactobacillus plantarum comprises a nucleic acid sequence with at least about 70% sequence identity to SEQ ID NO: 6, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 6, including all subranges and values that lie therebetween.

In some embodiments, the VR6 of Lactobacillus plantarum comprises a nucleic acid sequence with at least about 70% sequence identity to SEQ ID NO: 7, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 7, including all subranges and values that lie therebetween.

In some embodiments, the VR7 of Lactobacillus plantarum comprises a nucleic acid sequence with at least about 70% sequence identity to SEQ ID NO: 8, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 8, including all subranges and values that lie therebetween.

In some embodiments, the VR8 of Lactobacillus plantarum comprises a nucleic acid sequence with at least about 70% sequence identity to SEQ ID NO: 9, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 9, including all subranges and values that lie therebetween.

In some embodiments, the VR9 of Lactobacillus plantarum comprises a nucleic acid sequence with at least about 70% sequence identity to SEQ ID NO: 10, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 10, including all subranges and values that lie therebetween.

In some embodiments, the CR1 of Lactobacillus plantarum comprises a nucleic acid sequence with at least about 70% sequence identity to SEQ ID NO: 11, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 11, including all subranges and values that lie therebetween.

In some embodiments, the CR2 of Lactobacillus plantarum comprises a nucleic acid sequence with at least about 70% sequence identity to SEQ ID NO: 12, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 12, including all subranges and values that lie therebetween.

In some embodiments, the CR3 of Lactobacillus plantarum comprises a nucleic acid sequence with at least about 70% sequence identity to SEQ ID NO: 13, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at 99.3%, at least about 99.4%, at least about 99.5%, at least about 99.6%, at least about 99.7%, at least about 99.8%, at least about 99.9%, or about 100% sequence identity to SEQ ID NO: 13, including all subranges and values that lie therebetween.

In some embodiments, the CR4 of Lactobacillus plantarum comprises a nucleic acid sequence with at least about 70% sequence identity to SEQ ID NO: 14, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 14, including all subranges and values that lie therebetween.

In some embodiments, the CR5 of Lactobacillus plantarum comprises a nucleic acid sequence with at least about 70% sequence identity to SEQ ID NO: 15, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 15, including all subranges and values that lie therebetween.

In some embodiments, the CR6 of Lactobacillus plantarum comprises a nucleic acid sequence with at least about 70% sequence identity to SEQ ID NO: 16, for example, at least about 70%, at least about 75%, at least about 80%, at least about 85%, at least about least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 16, including all subranges and values that lie therebetween.

In some embodiments, the CR7 of Lactobacillus plantarum comprises a nucleic acid sequence with at least about 70% sequence identity to SEQ ID NO: 17, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 17, including all subranges and values that lie therebetween.

In some embodiments, the CR8 of Lactobacillus plantarum comprises a nucleic acid sequence with at least about 70% sequence identity to SEQ ID NO: 18, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least 99.7%, at least about 99.8%, at least about 99.9%, or about 100% sequence identity to SEQ ID NO: 18, including all subranges and values that lie therebetween.

In some embodiments, the CR9 of Lactobacillus plantarum comprises a nucleic acid sequence with at least about 70% sequence identity to SEQ ID NO: 19, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 19, including all subranges and values that lie therebetween.

In some embodiments, the CR10 of Lactobacillus plantarum comprises a nucleic acid sequence with at least about 70% sequence identity to SEQ ID NO: 20, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 20, including all subranges and values that lie therebetween.

In some embodiments, the strain of Lactobacillus plantarum comprises a 16S rRNA gene comprising a nucleic acid sequence comprising at least about 70% sequence identity to SEQ ID NO: 1, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 1, including all subranges and values that lie therebetween.

The disclosure further provides an isolated and purified Lactobacillus plantarum having deposit accession number 202103009, or a strain having all of the identifying characteristics of Lactobacillus plantarum 202103009, or a mutant thereof.

Lactobacillus plantarum may be interchangeably referred to as Lactiplantibacillus plantarum throughout the present disclosure.

(ii) Lactobacillus rhamnosus Strains

In some embodiments, the athlete-associated gut microbe comprises a strain of Lactobacillus rhamnosus. In some embodiments, the VR1 of Lactobacillus rhamnosus comprises a nucleic acid sequence with at least about 70% sequence identity to SEQ ID NO: 22, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 22, including all subranges and values that lie therebetween.

In some embodiments, the VR2 of Lactobacillus rhamnosus comprises a nucleic acid sequence with at least about 70% sequence identity to SEQ ID NO: 23, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 23, including all subranges and values that lie therebetween.

In some embodiments, the VR3 of Lactobacillus rhamnosus comprises a nucleic acid sequence with at least about 70% sequence identity to SEQ ID NO: 24, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 24, including all subranges and values that lie therebetween.

In some embodiments, the VR4 of Lactobacillus rhamnosus comprises a nucleic acid sequence with at least about 70% sequence identity to SEQ ID NO: 25, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least 99.7%, at least about 99.8%, at least about 99.9%, or about 100% sequence identity to SEQ ID NO: 25, including all subranges and values that lie therebetween.

In some embodiments, the VR5 of Lactobacillus rhamnosus comprises a nucleic acid sequence with at least about 70% sequence identity to SEQ ID NO: 26, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 26, including all subranges and values that lie therebetween.

In some embodiments, the VR6 of Lactobacillus rhamnosus comprises a nucleic acid sequence with at least about 70% sequence identity to SEQ ID NO: 27, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 27, including all subranges and values that lie therebetween.

In some embodiments, the VR7 of Lactobacillus rhamnosus comprises a nucleic acid sequence with at least about 70% sequence identity to SEQ ID NO: 28, for example, at least about 70%, at least about 75%, at least about 80%, at least about 85%, at least about about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 28, including all subranges and values that lie therebetween.

In some embodiments, the VR8 of Lactobacillus rhamnosus comprises a nucleic acid sequence with at least about 70% sequence identity to SEQ ID NO: 29, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 29, including all subranges and values that lie therebetween.

In some embodiments, the VR9 of Lactobacillus rhamnosus comprises a nucleic acid sequence with at least about 70% sequence identity to SEQ ID NO: 30, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about ID NO: 30, including all subranges and values that lie therebetween.

In some embodiments, the CR1 of Lactobacillus rhamnosus comprises a nucleic acid sequence with at least about 70% sequence identity to SEQ ID NO: 31, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 31, including all subranges and values that lie therebetween.

In some embodiments, the CR2 of Lactobacillus rhamnosus comprises a nucleic acid sequence with at least about 70% sequence identity to SEQ ID NO: 32, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 32, including all subranges and values that lie therebetween.

In some embodiments, the CR3 of Lactobacillus rhamnosus comprises a nucleic acid sequence with at least about 70% sequence identity to SEQ ID NO: 33, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 33, including all subranges and values that lie therebetween.

In some embodiments, the CR4 of Lactobacillus rhamnosus comprises a nucleic acid sequence with at least about 70% sequence identity to SEQ ID NO: 34, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 34, including all subranges and values that lie therebetween.

In some embodiments, the CR5 of Lactobacillus rhamnosus comprises a nucleic acid sequence with at least about 70% sequence identity to SEQ ID NO: 35, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 35, including all subranges and values that lie therebetween.

In some embodiments, the CR6 of Lactobacillus rhamnosus comprises a nucleic acid sequence with at least about 70% sequence identity to SEQ ID NO: 36, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 36, including all subranges and values that lie therebetween.

In some embodiments, the CR7 of Lactobacillus rhamnosus comprises a nucleic acid sequence with at least about 70% sequence identity to SEQ ID NO: 37, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 37, including all subranges and values that lie therebetween.

In some embodiments, the CR8 of Lactobacillus rhamnosus comprises a nucleic acid sequence with at least about 70% sequence identity to SEQ ID NO: 38, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at 99.3%, at least about 99.4%, at least about 99.5%, at least about 99.6%, at least about 99.7%, at least about 99.8%, at least about 99.9%, or about 100% sequence identity to SEQ ID NO: 38, including all subranges and values that lie therebetween.

In some embodiments, the CR9 of Lactobacillus rhamnosus comprises a nucleic acid sequence with at least about 70% sequence identity to SEQ ID NO: 39, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 39, including all subranges and values that lie therebetween.

In some embodiments, the CR10 of Lactobacillus rhamnosus comprises a nucleic acid sequence with at least about 70% sequence identity to SEQ ID NO: 40, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 40, including all subranges and values that lie therebetween.

In some embodiments, the strain of Lactobacillus rhamnosus comprises a 16S rRNA gene comprising a nucleic acid sequence comprising at least about 70% sequence identity to SEQ ID NO: 21, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 21, including all subranges and values that lie therebetween.

The disclosure further provides an isolated and purified Lactobacillus rhamnosus having deposit accession number 202103010, or a strain having all of the identifying characteristics of Lactobacillus rhamnosus 202103010, or a mutant thereof.

Lactobacillus rhamnosus may be interchangeably referred to as Lacticaseibacillus rhamnosus throughout the present disclosure.

(iii) Lactobacillus acidophilus Strains

In some embodiments, the athlete-associated gut microbe comprises a strain of Lactobacillus acidophilus. In some embodiments, the VR1 of Lactobacillus acidophilus comprises a nucleic acid sequence with at least about 70% sequence identity to SEQ ID NO: 42, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 42, including all subranges and values that lie therebetween.

In some embodiments, the VR2 of Lactobacillus acidophilus comprises a nucleic acid sequence with at least about 70% sequence identity to SEQ ID NO: 43, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 43, including all subranges and values that lie therebetween.

In some embodiments, the VR3 of Lactobacillus acidophilus comprises a nucleic acid sequence with at least about 70% sequence identity to SEQ ID NO: 44, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 44, including all subranges and values that lie therebetween.

In some embodiments, the VR4 of Lactobacillus acidophilus comprises a nucleic acid sequence with at least about 70% sequence identity to SEQ ID NO: 45, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at 99.3%, at least about 99.4%, at least about 99.5%, at least about 99.6%, at least about 99.7%, at least about 99.8%, at least about 99.9%, or about 100% sequence identity to SEQ ID NO: 45, including all subranges and values that lie therebetween.

In some embodiments, the VR5 of Lactobacillus acidophilus comprises a nucleic acid sequence with at least about 70% sequence identity to SEQ ID NO: 46, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 46, including all subranges and values that lie therebetween.

In some embodiments, the VR6 of Lactobacillus acidophilus comprises a nucleic acid sequence with at least about 70% sequence identity to SEQ ID NO: 47, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 47, including all subranges and values that lie therebetween.

In some embodiments, the VR7 of Lactobacillus acidophilus comprises a nucleic acid sequence with at least about 70% sequence identity to SEQ ID NO: 48, for example, at least about 70%, at least about 75%, at least about 80%, at least about 85%, at least about least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 48, including all subranges and values that lie therebetween.

In some embodiments, the VR8 of Lactobacillus acidophilus comprises a nucleic acid sequence with at least about 70% sequence identity to SEQ ID NO: 49, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 49, including all subranges and values that lie therebetween.

In some embodiments, the VR9 of Lactobacillus acidophilus comprises a nucleic acid sequence with at least about 70% sequence identity to SEQ ID NO: 50, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least 99.7%, at least about 99.8%, at least about 99.9%, or about 100% sequence identity to SEQ ID NO: 50, including all subranges and values that lie therebetween.

In some embodiments, the CR1 of Lactobacillus acidophilus comprises a nucleic acid sequence with at least about 70% sequence identity to SEQ ID NO: 51, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 51, including all subranges and values that lie therebetween.

In some embodiments, the CR2 of Lactobacillus acidophilus comprises a nucleic acid sequence with at least about 70% sequence identity to SEQ ID NO: 52, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 52, including all subranges and values that lie therebetween.

In some embodiments, the CR3 of Lactobacillus acidophilus comprises a nucleic acid sequence with at least about 70% sequence identity to SEQ ID NO: 53, for example, at least about 70%, at least about 75%, at least about 80%, at least about 85%, at least about about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 53, including all subranges and values that lie therebetween.

In some embodiments, the CR4 of Lactobacillus acidophilus comprises a nucleic acid sequence with at least about 70% sequence identity to SEQ ID NO: 54, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 54, including all subranges and values that lie therebetween.

In some embodiments, the CR5 of Lactobacillus acidophilus comprises a nucleic acid sequence with at least about 70% sequence identity to SEQ ID NO: 55, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about ID NO: 55, including all subranges and values that lie therebetween.

In some embodiments, the CR6 of Lactobacillus acidophilus comprises a nucleic acid sequence with at least about 70% sequence identity to SEQ ID NO: 56, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 56, including all subranges and values that lie therebetween.

In some embodiments, the CR7 of Lactobacillus acidophilus comprises a nucleic acid sequence with at least about 70% sequence identity to SEQ ID NO: 57, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 57, including all subranges and values that lie therebetween.

In some embodiments, the CR8 of Lactobacillus acidophilus comprises a nucleic acid sequence with at least about 70% sequence identity to SEQ ID NO: 58, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 58, including all subranges and values that lie therebetween.

In some embodiments, the CR9 of Lactobacillus acidophilus comprises a nucleic acid sequence with at least about 70% sequence identity to SEQ ID NO: 59, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 59, including all subranges and values that lie therebetween.

In some embodiments, the CR10 of Lactobacillus acidophilus comprises a nucleic acid sequence with at least about 70% sequence identity to SEQ ID NO: 60, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 60, including all subranges and values that lie therebetween.

In some embodiments, the strain of Lactobacillus acidophilus comprises a 16S rRNA gene comprising a nucleic acid sequence comprising at least about 70% sequence identity to SEQ ID NO: 41, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 41, including all subranges and values that lie therebetween.

The disclosure further provides an isolated and purified Lactobacillus acidophilus having deposit accession number 202103008, or a strain having all of the identifying characteristics of Lactobacillus acidophilus 202103008, or a mutant thereof.

(iv) Lactobacillus paracasei Strains

In some embodiments, the athlete-associated gut microbe comprises a strain of Lactobacillus paracasei. In some embodiments, the VR1 of Lactobacillus paracasei comprises a nucleic acid sequence with at least about 70% sequence identity to SEQ ID NO: 62 or SEQ ID NO: 182, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 62 or SEQ ID NO: 182, including all subranges and values that lie therebetween.

In some embodiments, the VR2 of Lactobacillus paracasei comprises a nucleic acid sequence with at least about 70% sequence identity to SEQ ID NO: 63 or SEQ ID NO:

183, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 63 or SEQ ID NO: 183, including all subranges and values that lie therebetween.

In some embodiments, the VR3 of Lactobacillus paracasei comprises a nucleic acid sequence with at least about 70% sequence identity to SEQ ID NO: 64 or SEQ ID NO: 184, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 64 or SEQ ID NO: 184, including all subranges and values that lie therebetween.

In some embodiments, the VR4 of Lactobacillus paracasei comprises a nucleic acid sequence with at least about 70% sequence identity to SEQ ID NO: 65 or SEQ ID NO: 185, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 65 or SEQ ID NO: 185, including all subranges and values that lie therebetween.

In some embodiments, the VR5 of Lactobacillus paracasei comprises a nucleic acid sequence with at least about 70% sequence identity to SEQ ID NO: 66 or SEQ ID NO: 186, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 66 or SEQ ID NO: 186, including all subranges and values that lie therebetween.

In some embodiments, the VR6 of Lactobacillus paracasei comprises a nucleic acid sequence with at least about 70% sequence identity to SEQ ID NO: 67 or SEQ ID NO: 187, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 67 or SEQ ID NO: 187, including all subranges and values that lie therebetween.

In some embodiments, the VR7 of Lactobacillus paracasei comprises a nucleic acid sequence with at least about 70% sequence identity to SEQ ID NO: 68 or SEQ ID NO: 188, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 68 or SEQ ID NO: 188, including all subranges and values that lie therebetween.

In some embodiments, the VR8 of Lactobacillus paracasei comprises a nucleic acid sequence with at least about 70% sequence identity to SEQ ID NO: 69 or SEQ ID NO: 189, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 69 or SEQ ID NO: 189, including all subranges and values that lie therebetween.

In some embodiments, the VR9 of Lactobacillus paracasei comprises a nucleic acid sequence with at least about 70% sequence identity to SEQ ID NO: 70 or SEQ ID NO: 190, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 70 or SEQ ID NO: 190, including all subranges and values that lie therebetween.

In some embodiments, the CR1 of Lactobacillus paracasei comprises a nucleic acid sequence with at least about 70% sequence identity to SEQ ID NO: 71 or SEQ ID NO: 191, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 71 or SEQ ID NO: 191, including all subranges and values that lie therebetween.

In some embodiments, the CR2 of Lactobacillus paracasei comprises a nucleic acid sequence with at least about 70% sequence identity to SEQ ID NO: 72 or SEQ ID NO: 192, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 72 or SEQ ID NO: 192, including all subranges and values that lie therebetween.

In some embodiments, the CR3 of Lactobacillus paracasei comprises a nucleic acid sequence with at least about 70% sequence identity to SEQ ID NO: 73 or SEQ ID NO: 193, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 73 or SEQ ID NO: 193, including all subranges and values that lie therebetween.

In some embodiments, the CR4 of Lactobacillus paracasei comprises a nucleic acid sequence with at least about 70% sequence identity to SEQ ID NO: 74 or SEQ ID NO: 194, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 74 or SEQ ID NO: 194, including all subranges and values that lie therebetween.

In some embodiments, the CR5 of Lactobacillus paracasei comprises a nucleic acid sequence with at least about 70% sequence identity to SEQ ID NO: 75 or SEQ ID NO: 195, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 75 or SEQ ID NO: 195, including all subranges and values that lie therebetween.

In some embodiments, the CR6 of Lactobacillus paracasei comprises a nucleic acid sequence with at least about 70% sequence identity to SEQ ID NO: 76 or SEQ ID NO: 196, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 76 or SEQ ID NO: 196, including all subranges and values that lie therebetween.

In some embodiments, the CR7 of Lactobacillus paracasei comprises a nucleic acid sequence with at least about 70% sequence identity to SEQ ID NO: 77 or SEQ ID NO: 197, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 77 or SEQ ID NO: 197, including all subranges and values that lie therebetween.

In some embodiments, the CR8 of Lactobacillus paracasei comprises a nucleic acid sequence with at least about 70% sequence identity to SEQ ID NO: 78 or SEQ ID NO: 198, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 78 or SEQ ID NO: 198, including all subranges and values that lie therebetween.

In some embodiments, the CR9 of Lactobacillus paracasei comprises a nucleic acid sequence with at least about 70% sequence identity to SEQ ID NO: 79 or SEQ ID NO: 199, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 79 or SEQ ID NO: 199, including all subranges and values that lie therebetween.

In some embodiments, the CR10 of Lactobacillus paracasei comprises a nucleic acid sequence with at least about 70% sequence identity to SEQ ID NO: 80 or SEQ ID NO: 200, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 80 or SEQ ID NO: 200, including all subranges and values that lie therebetween.

In some embodiments, the strain of Lactobacillus paracasei comprises a 16S rRNA gene comprising a nucleic acid sequence comprising at least about 70% sequence identity to SEQ ID NO: 61, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 61, including all subranges and values that lie therebetween.

The disclosure further provides an isolated and purified Lactobacillus paracasei having deposit accession number 202103012, or a strain having all of the identifying characteristics of Lactobacillus paracasei 202103012, or a mutant thereof.

In some embodiments, the strain of Lactobacillus paracasei comprises a 16S rRNA gene comprising a nucleic acid sequence comprising at least about 70% sequence identity to SEQ ID NO: 181, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 181, including all subranges and values that lie therebetween.

The disclosure further provides an isolated and purified Lactobacillus paracasei having deposit accession number 202103018, or a strain having all of the identifying characteristics of Lactobacillus paracasei 202103018, or a mutant thereof.

### (b) Athlete-Associated Bifidobacterium Strains

In some embodiments, the athlete-associated gut microbe comprises a microbial strain belonging to the genus Bifidobacterium. In some embodiments, the microbial strain belonging to the genus Bifidobacterium is an isolated and purified strain. In some embodiments, the microbial strain belonging to the genus Bifidobacterium comprises a 16S ribosomal RNA (rRNA) gene comprising a nucleic acid sequence comprising at least one variable region. In some embodiments, the microbial strain belonging to the genus Bifidobacterium comprises a 16S rRNA gene comprising a nucleic acid sequence comprising at least one constant region. In some embodiments, the at least one variable region (VR) is selected from the group consisting of: VR1, VR2, VR3, VR4, VR5, VR6, VR7, VR8, VR9, and any combination thereof. In some embodiments, the at least one constant region (CR) selected from the group consisting of: CR1, CR2, CR3, CR4, CR5, CR6, CR7, CR8, CR9, CR10, and any combination thereof.

In some embodiments, the microbial strain belonging to the genus Bifidobacterium comprises a 16S rRNA gene comprising a nucleic acid sequence comprising the following variable regions: VR1, VR2, VR3, VR4, VR5, VR6, VR7, VR8, and VR9, and the following constant regions: CR1, CR2, CR3, CR4, CR5, CR6, CR7, CR8, CR9, CR10. In some embodiments, the VRs and CRs are ordered as shown below: 5′-CR1-VR1-CR2-VR2-CR3-VR3-CR4-VR4-CR5-VR5-CR6-VR6-CR7-VR7-CR8-VR8-CR9-VR9-CR10-3′

(i) Bifidobacterium longum Strains

In some embodiments, the athlete-associated gut microbe comprises a strain of Bifidobacterium longum. In some embodiments, the VR1 of Bifidobacterium longum comprises a nucleic acid sequence with at least about 70% sequence identity to SEQ ID NO: 82, SEQ ID NO: 202, or SEQ ID NO: 222, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 82, SEQ ID NO: 202, or SEQ ID NO: 222, including all subranges and values that lie therebetween.

In some embodiments, the VR2 of Bifidobacterium longum comprises a nucleic acid sequence with at least about 70% sequence identity to SEQ ID NO: 83, SEQ ID NO: 203, or SEQ ID NO: 223, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 83, SEQ ID NO: 203, or SEQ ID NO: 223, including all subranges and values that lie therebetween.

In some embodiments, the VR3 of Bifidobacterium longum comprises a nucleic acid sequence with at least about 70% sequence identity to SEQ ID NO: 84, SEQ ID NO: 204, or SEQ ID NO: 224, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 84, SEQ ID NO: 204, or SEQ ID NO: 224, including all subranges and values that lie therebetween.

In some embodiments, the VR4 of Bifidobacterium longum comprises a nucleic acid sequence with at least about 70% sequence identity to SEQ ID NO: 85, SEQ ID NO: 205, or SEQ ID NO: 225, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 85, SEQ ID NO: 205, or SEQ ID NO: 225, including all subranges and values that lie therebetween.

In some embodiments, the VR5 of Bifidobacterium longum comprises a nucleic acid sequence with at least about 70% sequence identity to SEQ ID NO: 86, SEQ ID NO: 206, or SEQ ID NO: 226, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 86, SEQ ID NO: 206, or SEQ ID NO: 226, including all subranges and values that lie therebetween.

In some embodiments, the VR6 of Bifidobacterium longum comprises a nucleic acid sequence with at least about 70% sequence identity to SEQ ID NO: 87, SEQ ID NO: 207, or SEQ ID NO: 227, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 87, SEQ ID NO: 207, or SEQ ID NO: 227, including all subranges and values that lie therebetween.

In some embodiments, the VR7 of Bifidobacterium longum comprises a nucleic acid sequence with at least about 70% sequence identity to SEQ ID NO: 88, SEQ ID NO: 208, or SEQ ID NO: 228, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 88, SEQ ID NO: 208, or SEQ ID NO: 228,including all subranges and values that lie therebetween.

In some embodiments, the VR8 of Bifidobacterium longum comprises a nucleic acid sequence with at least about 70% sequence identity to SEQ ID NO: 89, SEQ ID NO: 209, or SEQ ID NO: 229, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 89, SEQ ID NO: 209, or SEQ ID NO: 229, including all subranges and values that lie therebetween.

In some embodiments, the VR9 of Bifidobacterium longum comprises a nucleic acid sequence with at least about 70% sequence identity to SEQ ID NO: 90, SEQ ID NO: 210, or SEQ ID NO: 230, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 90, SEQ ID NO: 210, or SEQ ID NO: 230, including all subranges and values that lie therebetween.

In some embodiments, the CR1 of Bifidobacterium longum comprises a nucleic acid sequence with at least about 70% sequence identity to SEQ ID NO: 91, SEQ ID NO: 211, or SEQ ID NO: 231, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 91, SEQ ID NO: 211, or SEQ ID NO: 231, including all subranges and values that lie therebetween.

In some embodiments, the CR2 of Bifidobacterium longum comprises a nucleic acid sequence with at least about 70% sequence identity to SEQ ID NO: 92, SEQ ID NO: 212, or SEQ ID NO: 232, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 92, SEQ ID NO: 212, or SEQ ID NO: 232, including all subranges and values that lie therebetween.

In some embodiments, the CR3 of Bifidobacterium longum comprises a nucleic acid sequence with at least about 70% sequence identity to SEQ ID NO: 93, SEQ ID NO: 213, or SEQ ID NO: 233, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 93, SEQ ID NO: 213, or SEQ ID NO: 233, including all subranges and values that lie therebetween.

In some embodiments, the CR4 of Bifidobacterium longum comprises a nucleic acid sequence with at least about 70% sequence identity to SEQ ID NO: 94, SEQ ID NO: 214, or SEQ ID NO: 234, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 94, SEQ ID NO: 214, or SEQ ID NO: 234, including all subranges and values that lie therebetween.

In some embodiments, the CR5 of Bifidobacterium longum comprises a nucleic acid sequence with at least about 70% sequence identity to SEQ ID NO: 95, SEQ ID NO: 215, or SEQ ID NO: 235, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 95, SEQ ID NO: 215, or SEQ ID NO: 235, including all subranges and values that lie therebetween.

In some embodiments, the CR6 of Bifidobacterium longum comprises a nucleic acid sequence with at least about 70% sequence identity to SEQ ID NO: 96, SEQ ID NO: 216, or SEQ ID NO: 236, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 96, SEQ ID NO: 216, or SEQ ID NO: 236, including all subranges and values that lie therebetween.

In some embodiments, the CR7 of Bifidobacterium longum comprises a nucleic acid sequence with at least about 70% sequence identity to SEQ ID NO: 97, SEQ ID NO: 217, or SEQ ID NO: 237, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 97, SEQ ID NO: 217, or SEQ ID NO: 237, including all subranges and values that lie therebetween.

In some embodiments, the CR8 of Bifidobacterium longum comprises a nucleic acid sequence with at least about 70% sequence identity to SEQ ID NO: 98, SEQ ID NO: 218, or SEQ ID NO: 238, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 98, SEQ ID NO: 218, or SEQ ID NO: 238, including all subranges and values that lie therebetween.

In some embodiments, the CR9 of Bifidobacterium longum comprises a nucleic acid sequence with at least about 70% sequence identity to SEQ ID NO: 99, SEQ ID NO: 219, or SEQ ID NO: 239, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 99, SEQ ID NO: 219, or SEQ ID NO: 239, including all subranges and values that lie therebetween.

In some embodiments, the CR10 of Bifidobacterium longum comprises a nucleic acid sequence with at least about 70% sequence identity to SEQ ID NO: 100, SEQ ID NO: 220, or SEQ ID NO: 240, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 100, SEQ ID NO: 220, or SEQ ID NO: 240, including all subranges and values that lie therebetween.

In some embodiments, the strain of Bifidobacterium longum comprises a 16S rRNA gene comprising a nucleic acid sequence comprising at least about 70% sequence identity to SEQ ID NO: 81, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 81, including all subranges and values that lie therebetween.

The disclosure further provides an isolated and purified Bifidobacterium longum having deposit accession number 202103014, or a strain having all of the identifying characteristics of Bifidobacterium longum 202103014, or a mutant thereof.

In some embodiments, the strain of Bifidobacterium longum comprises a 16S rRNA gene comprising a nucleic acid sequence comprising at least about 70% sequence identity to SEQ ID NO: 201, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 201, including all subranges and values that lie therebetween.

The disclosure further provides an isolated and purified Bifidobacterium longum having deposit accession number 202103013, or a strain having all of the identifying characteristics of Bifidobacterium longum 202103013, or a mutant thereof.

In some embodiments, the strain of Bifidobacterium longum comprises a 16S rRNA gene comprising a nucleic acid sequence comprising at least about 70% sequence identity to SEQ ID NO: 221, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 221, including all subranges and values that lie therebetween.

The disclosure further provides an isolated and purified Bifidobacterium longum having deposit accession number 202103015, or a strain having all of the identifying characteristics of Bifidobacterium longum 202103015, or a mutant thereof.

(ii) Bifidobacterium animalis lactis Strains

In some embodiments, the athlete-associated gut microbe comprises a strain of Bifidobacterium animalis lactis. In some embodiments, the VR1 of Bifidobacterium animalis lactis comprises a nucleic acid sequence with at least about 70% sequence identity to SEQ ID NO: 102, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 102, including all subranges and values that lie therebetween.

In some embodiments, the VR2 of Bifidobacterium animalis lactis comprises a nucleic acid sequence with at least about 70% sequence identity to SEQ ID NO: 103, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 103, including all subranges and values that lie therebetween.

In some embodiments, the VR3 of Bifidobacterium animalis lactis comprises a nucleic acid sequence with at least about 70% sequence identity to SEQ ID NO: 104, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 104, including all subranges and values that lie therebetween.

In some embodiments, the VR4 of Bifidobacterium animalis lactis comprises a nucleic acid sequence with at least about 70% sequence identity to SEQ ID NO: 105, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 105, including all subranges and values that lie therebetween.

In some embodiments, the VR5 of Bifidobacterium animalis lactis comprises a nucleic acid sequence with at least about 70% sequence identity to SEQ ID NO: 106, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 106, including all subranges and values that lie therebetween.

In some embodiments, the VR6 of Bifidobacterium animalis lactis comprises a nucleic acid sequence with at least about 70% sequence identity to SEQ ID NO: 107, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 107, including all subranges and values that lie therebetween.

In some embodiments, the VR7 of Bifidobacterium animalis lactis comprises a nucleic acid sequence with at least about 70% sequence identity to SEQ ID NO: 108, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 108, including all subranges and values that lie therebetween.

In some embodiments, the VR8 of Bifidobacterium animalis lactis comprises a nucleic acid sequence with at least about 70% sequence identity to SEQ ID NO: 109, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 109, including all subranges and values that lie therebetween.

In some embodiments, the VR9 of Bifidobacterium animalis lactis comprises a nucleic acid sequence with at least about 70% sequence identity to SEQ ID NO: 110, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 110, including all subranges and values that lie therebetween.

In some embodiments, the CR1 of Bifidobacterium animalis lactis comprises a nucleic acid sequence with at least about 70% sequence identity to SEQ ID NO: 111, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 111, including all subranges and values that lie therebetween.

In some embodiments, the CR2 of Bifidobacterium animalis lactis comprises a nucleic acid sequence with at least about 70% sequence identity to SEQ ID NO: 112, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 112, including all subranges and values that lie therebetween.

In some embodiments, the CR3 of Bifidobacterium animalis lactis comprises a nucleic acid sequence with at least about 70% sequence identity to SEQ ID NO: 113, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 113, including all subranges and values that lie therebetween.

In some embodiments, the CR4 of Bifidobacterium animalis lactis comprises a nucleic acid sequence with at least about 70% sequence identity to SEQ ID NO: 114, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 114, including all subranges and values that lie therebetween.

In some embodiments, the CR5 of Bifidobacterium animalis lactis comprises a nucleic acid sequence with at least about 70% sequence identity to SEQ ID NO: 115, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 115, including all subranges and values that lie therebetween.

In some embodiments, the CR6 of Bifidobacterium animalis lactis comprises a nucleic acid sequence with at least about 70% sequence identity to SEQ ID NO: 116, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 116, including all subranges and values that lie therebetween.

In some embodiments, the CR7 of Bifidobacterium animalis lactis comprises a nucleic acid sequence with at least about 70% sequence identity to SEQ ID NO: 117, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 117, including all subranges and values that lie therebetween.

In some embodiments, the CR8 of Bifidobacterium animalis lactis comprises a nucleic acid sequence with at least about 70% sequence identity to SEQ ID NO: 118, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 118, including all subranges and values that lie therebetween.

In some embodiments, the CR9 of Bifidobacterium animalis lactis comprises a nucleic acid sequence with at least about 70% sequence identity to SEQ ID NO: 119, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 119, including all subranges and values that lie therebetween.

In some embodiments, the CR10 of Bifidobacterium animalis lactis comprises a nucleic acid sequence with at least about 70% sequence identity to SEQ ID NO: 120, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 120, including all subranges and values that lie therebetween.

In some embodiments, the strain of Bifidobacterium animalis lactis comprises a 16S rRNA gene comprising a nucleic acid sequence comprising at least about 70% sequence identity to SEQ ID NO: 101, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 101, including all subranges and values that lie therebetween.

The disclosure further provides an isolated and purified Bifidobacterium animalis lactis having deposit accession number 202103016, or a strain having all of the identifying characteristics of Bifidobacterium animalis lactis 202103016, or a mutant thereof.

The disclosure further provides an isolated and purified Bifidobacterium animalis lactis having deposit accession number 202103017, or a strain having all of the identifying characteristics of Bifidobacterium animalis lactis 202103017, or a mutant thereof.

The disclosure further provides an isolated and purified Bifidobacterium animalis lactis having deposit accession number 202103019, or a strain having all of the identifying characteristics of Bifidobacterium animalis lactis 202103019, or a mutant thereof.

### (c) Athlete-Associated Veillonella Strains

In some embodiments, the athlete-associated gut microbe comprises a microbial strain belonging to the genus Veillonella. In some embodiments, the microbial strain belonging to the genus Veillonella is an isolated and purified strain. In some embodiments, the microbial strain belonging to the genus Veillonella comprises a 16S ribosomal RNA (rRNA) gene comprising a nucleic acid sequence comprising at least one variable region. In some embodiments, the microbial strain belonging to the genus Veillonella comprises a 16S rRNA gene comprising a nucleic acid sequence comprising at least one constant region. In some embodiments, the at least one variable region (VR) is selected from the group consisting of: VR1, VR2, VR3, VR4, VR5, VR6, VR7, VR8, VR9, and any combination thereof. In some embodiments, the at least one constant region (CR) selected from the group consisting of: CR1, CR2, CR3, CR4, CR5, CR6, CR7, CR8, CR9, CR10, and any combination thereof.

In some embodiments, the microbial strain belonging to the genus Veillonella comprises a 16S rRNA gene comprising a nucleic acid sequence comprising the following variable regions: VR1, VR2, VR3, VR4, VR5, VR6, VR7, VR8, and VR9, and the following constant regions: CR1, CR2, CR3, CR4, CR5, CR6, CR7, CR8, CR9, CR10. In some embodiments, the VRs and CRs are ordered as shown below: 5′-CR1-VR1-CR2-VR2-CR3-VR3-CR4-VR4-CR5-VR5-CR6-VR6-CR7-VR7-CR8-VR8-CR9-VR9-CR10-3′

(i) Veillonella dispar Strains

In some embodiments, the athlete-associated gut microbe comprises a strain of Veillonella dispar. In some embodiments, the VR1 of Veillonella dispar comprises a nucleic acid sequence with at least about 70% sequence identity to SEQ ID NO: 122, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 122, including all subranges and values that lie therebetween.

In some embodiments, the VR2 of Veillonella dispar comprises a nucleic acid sequence with at least about 70% sequence identity to SEQ ID NO: 123, for example, at least about 70%, at least about 75%, at least about 80%, at least about 85%, at least about 90%, at least about 95%, at least about 96%, at least about 97.0%, at least about 97.1%, at 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 123, including all subranges and values that lie therebetween.

In some embodiments, the VR3 of Veillonella dispar comprises a nucleic acid sequence with at least about 70% sequence identity to SEQ ID NO: 124, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 124, including all subranges and values that lie therebetween.

In some embodiments, the VR4 of Veillonella dispar comprises a nucleic acid sequence with at least about 70% sequence identity to SEQ ID NO: 125, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about ID NO: 125, including all subranges and values that lie therebetween.

In some embodiments, the VR5 of Veillonella dispar comprises a nucleic acid sequence with at least about 70% sequence identity to SEQ ID NO: 126, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 126, including all subranges and values that lie therebetween.

In some embodiments, the VR6 of Veillonella dispar comprises a nucleic acid sequence with at least about 70% sequence identity to SEQ ID NO: 127, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 127, including all subranges and values that lie therebetween.

In some embodiments, the VR7 of Veillonella dispar comprises a nucleic acid sequence with at least about 70% sequence identity to SEQ ID NO: 128, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 128, including all subranges and values that lie therebetween.

In some embodiments, the VR8 of Veillonella dispar comprises a nucleic acid sequence with at least about 70% sequence identity to SEQ ID NO: 129, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 129, including all subranges and values that lie therebetween.

In some embodiments, the VR9 of Veillonella dispar comprises a nucleic acid sequence with at least about 70% sequence identity to SEQ ID NO: 130, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 130, including all subranges and values that lie therebetween.

In some embodiments, the CR1 of Veillonella dispar comprises a nucleic acid sequence with at least about 70% sequence identity to SEQ ID NO: 131, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 131, including all subranges and values that lie therebetween.

In some embodiments, the CR2 of Veillonella dispar comprises a nucleic acid sequence with at least about 70% sequence identity to SEQ ID NO: 132, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 132, including all subranges and values that lie therebetween.

In some embodiments, the CR3 of Veillonella dispar comprises a nucleic acid sequence with at least about 70% sequence identity to SEQ ID NO: 133, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at 99.3%, at least about 99.4%, at least about 99.5%, at least about 99.6%, at least about 99.7%, at least about 99.8%, at least about 99.9%, or about 100% sequence identity to SEQ ID NO: 133, including all subranges and values that lie therebetween.

In some embodiments, the CR4 of Veillonella dispar comprises a nucleic acid sequence with at least about 70% sequence identity to SEQ ID NO: 134, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 134, including all subranges and values that lie therebetween.

In some embodiments, the CR5 of Veillonella dispar comprises a nucleic acid sequence with at least about 70% sequence identity to SEQ ID NO: 135, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 135, including all subranges and values that lie therebetween.

In some embodiments, the CR6 of Veillonella dispar comprises a nucleic acid sequence with at least about 70% sequence identity to SEQ ID NO: 136, for example, at least about 70%, at least about 75%, at least about 80%, at least about 85%, at least about least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 136, including all subranges and values that lie therebetween.

In some embodiments, the CR7 of Veillonella dispar comprises a nucleic acid sequence with at least about 70% sequence identity to SEQ ID NO: 137, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 137, including all subranges and values that lie therebetween.

In some embodiments, the CR8 of Veillonella dispar comprises a nucleic acid sequence with at least about 70% sequence identity to SEQ ID NO: 138, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least 99.7%, at least about 99.8%, at least about 99.9%, or about 100% sequence identity to SEQ ID NO: 138, including all subranges and values that lie therebetween.

In some embodiments, the CR9 of Veillonella dispar comprises a nucleic acid sequence with at least about 70% sequence identity to SEQ ID NO: 139, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 139, including all subranges and values that lie therebetween.

In some embodiments, the CR10 of Veillonella dispar comprises a nucleic acid sequence with at least about 70% sequence identity to SEQ ID NO: 140, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 140, including all subranges and values that lie therebetween.

In some embodiments, the strain of Veillonella dispar comprises a 16S rRNA gene comprising a nucleic acid sequence comprising at least about 70% sequence identity to SEQ ID NO: 121, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 121, including all subranges and values that lie therebetween.

The disclosure further provides an isolated and purified Veillonella dispar having deposit accession number PTA-126861, or a strain having all of the identifying characteristics of Veillonella dispar PTA-126861, or a mutant thereof.

(ii) Veillonella parvula Strains

In some embodiments, the athlete-associated gut microbe comprises a strain of Veillonella parvula. In some embodiments, the VR1 of Veillonella parvula comprises a nucleic acid sequence with at least about 70% sequence identity to SEQ ID NO: 142, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 142, including all subranges and values that lie therebetween.

In some embodiments, the VR2 of Veillonella parvula comprises a nucleic acid sequence with at least about 70% sequence identity to SEQ ID NO: 143, for example, at least about 70%, at least about 75%, at least about 80%, at least about 85%, at least about about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 143, including all subranges and values that lie therebetween.

In some embodiments, the VR3 of Veillonella parvula comprises a nucleic acid sequence with at least about 70% sequence identity to SEQ ID NO: 144, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 144, including all subranges and values that lie therebetween.

In some embodiments, the VR4 of Veillonella parvula comprises a nucleic acid sequence with at least about 70% sequence identity to SEQ ID NO: 145, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about ID NO: 145, including all subranges and values that lie therebetween.

In some embodiments, the VR5 of Veillonella parvula comprises a nucleic acid sequence with at least about 70% sequence identity to SEQ ID NO: 146, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 146, including all subranges and values that lie therebetween.

In some embodiments, the VR6 of Veillonella parvula comprises a nucleic acid sequence with at least about 70% sequence identity to SEQ ID NO: 147, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 147, including all subranges and values that lie therebetween.

In some embodiments, the VR7 of Veillonella parvula comprises a nucleic acid sequence with at least about 70% sequence identity to SEQ ID NO: 148, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 148, including all subranges and values that lie therebetween.

In some embodiments, the VR8 of Veillonella parvula comprises a nucleic acid sequence with at least about 70% sequence identity to SEQ ID NO: 149, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 149, including all subranges and values that lie therebetween.

In some embodiments, the VR9 of Veillonella parvula comprises a nucleic acid sequence with at least about 70% sequence identity to SEQ ID NO: 150, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 150, including all subranges and values that lie therebetween.

In some embodiments, the CR1 of Veillonella parvula comprises a nucleic acid sequence with at least about 70% sequence identity to SEQ ID NO: 151, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 151, including all subranges and values that lie therebetween.

In some embodiments, the CR2 of Veillonella parvula comprises a nucleic acid sequence with at least about 70% sequence identity to SEQ ID NO: 152, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 152, including all subranges and values that lie therebetween.

In some embodiments, the CR3 of Veillonella parvula comprises a nucleic acid sequence with at least about 70% sequence identity to SEQ ID NO: 153, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at 99.3%, at least about 99.4%, at least about 99.5%, at least about 99.6%, at least about 99.7%, at least about 99.8%, at least about 99.9%, or about 100% sequence identity to SEQ ID NO: 153, including all subranges and values that lie therebetween.

In some embodiments, the CR4 of Veillonella parvula comprises a nucleic acid sequence with at least about 70% sequence identity to SEQ ID NO: 154, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 154, including all subranges and values that lie therebetween.

In some embodiments, the CR5 of Veillonella parvula comprises a nucleic acid sequence with at least about 70% sequence identity to SEQ ID NO: 155, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 155, including all subranges and values that lie therebetween.

In some embodiments, the CR6 of Veillonella parvula comprises a nucleic acid sequence with at least about 70% sequence identity to SEQ ID NO: 156, for example, at least about 70%, at least about 75%, at least about 80%, at least about 85%, at least about least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 156, including all subranges and values that lie therebetween.

In some embodiments, the CR7 of Veillonella parvula comprises a nucleic acid sequence with at least about 70% sequence identity to SEQ ID NO: 157, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 157, including all subranges and values that lie therebetween.

In some embodiments, the CR8 of Veillonella parvula comprises a nucleic acid sequence with at least about 70% sequence identity to SEQ ID NO: 158, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least 99.7%, at least about 99.8%, at least about 99.9%, or about 100% sequence identity to SEQ ID NO: 158, including all subranges and values that lie therebetween.

In some embodiments, the CR9 of Veillonella parvula comprises a nucleic acid sequence with at least about 70% sequence identity to SEQ ID NO: 159, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 159, including all subranges and values that lie therebetween.

In some embodiments, the CR10 of Veillonella parvula comprises a nucleic acid sequence with at least about 70% sequence identity to SEQ ID NO: 160, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 160, including all subranges and values that lie therebetween.

In some embodiments, the strain of Veillonella parvula comprises a 16S rRNA gene comprising a nucleic acid sequence comprising at least about 70% sequence identity to SEQ ID NO: 141, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 141, including all subranges and values that lie therebetween.

In addition, the disclosure provides an isolated and purified Veillonella parvula having deposit accession number PTA-126859, or a strain having all of the identifying characteristics of Veillonella parvula PTA-126859, or a mutant thereof.

(iii) Veillonella atypica Strains

In some embodiments, the athlete-associated gut microbe comprises a strain of Veillonella atypica. In some embodiments, the VR1 of Veillonella atypica comprises a nucleic acid sequence with at least about 70% sequence identity to SEQ ID NO: 162, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 162, including all subranges and values that lie therebetween.

In some embodiments, the VR2 of Veillonella atypica comprises a nucleic acid sequence with at least about 70% sequence identity to SEQ ID NO: 163, for example, at least about 70%, at least about 75%, at least about 80%, at least about 85%, at least about about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 163, including all subranges and values that lie therebetween.

In some embodiments, the VR3 of Veillonella atypica comprises a nucleic acid sequence with at least about 70% sequence identity to SEQ ID NO: 164, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 164, including all subranges and values that lie therebetween.

In some embodiments, the VR4 of Veillonella atypica comprises a nucleic acid sequence with at least about 70% sequence identity to SEQ ID NO: 165, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about ID NO: 165, including all subranges and values that lie therebetween.

In some embodiments, the VR5 of Veillonella atypica comprises a nucleic acid sequence with at least about 70% sequence identity to SEQ ID NO: 166, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 166, including all subranges and values that lie therebetween.

In some embodiments, the VR6 of Veillonella atypica comprises a nucleic acid sequence with at least about 70% sequence identity to SEQ ID NO: 167, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 167, including all subranges and values that lie therebetween.

In some embodiments, the VR7 of Veillonella atypica comprises a nucleic acid sequence with at least about 70% sequence identity to SEQ ID NO: 168, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 168, including all subranges and values that lie therebetween.

In some embodiments, the VR8 of Veillonella atypica comprises a nucleic acid sequence with at least about 70% sequence identity to SEQ ID NO: 169, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 169, including all subranges and values that lie therebetween.

In some embodiments, the VR9 of Veillonella atypica comprises a nucleic acid sequence with at least about 70% sequence identity to SEQ ID NO: 170, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 170, including all subranges and values that lie therebetween.

In some embodiments, the CR1 of Veillonella atypica comprises a nucleic acid sequence with at least about 70% sequence identity to SEQ ID NO: 171, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 171, including all subranges and values that lie therebetween.

In some embodiments, the CR2 of Veillonella atypica comprises a nucleic acid sequence with at least about 70% sequence identity to SEQ ID NO: 172, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 172, including all subranges and values that lie therebetween.

In some embodiments, the CR3 of Veillonella atypica comprises a nucleic acid sequence with at least about 70% sequence identity to SEQ ID NO: 173, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at 99.3%, at least about 99.4%, at least about 99.5%, at least about 99.6%, at least about 99.7%, at least about 99.8%, at least about 99.9%, or about 100% sequence identity to SEQ ID NO: 173, including all subranges and values that lie therebetween.

In some embodiments, the CR4 of Veillonella atypica comprises a nucleic acid sequence with at least about 70% sequence identity to SEQ ID NO: 174, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 174, including all subranges and values that lie therebetween.

In some embodiments, the CR5 of Veillonella atypica comprises a nucleic acid sequence with at least about 70% sequence identity to SEQ ID NO: 175, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 175, including all subranges and values that lie therebetween.

In some embodiments, the CR6 of Veillonella atypica comprises a nucleic acid sequence with at least about 70% sequence identity to SEQ ID NO: 176, for example, at least about 70%, at least about 75%, at least about 80%, at least about 85%, at least about least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 176, including all subranges and values that lie therebetween.

In some embodiments, the CR7 of Veillonella atypica comprises a nucleic acid sequence with at least about 70% sequence identity to SEQ ID NO: 177, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 177, including all subranges and values that lie therebetween.

In some embodiments, the CR8 of Veillonella atypica comprises a nucleic acid sequence with at least about 70% sequence identity to SEQ ID NO: 178, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least 99.7%, at least about 99.8%, at least about 99.9%, or about 100% sequence identity to SEQ ID NO: 178, including all subranges and values that lie therebetween.

In some embodiments, the CR9 of Veillonella atypica comprises a nucleic acid sequence with at least about 70% sequence identity to SEQ ID NO: 179, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 179, including all subranges and values that lie therebetween.

In some embodiments, the CR10 of Veillonella atypica comprises a nucleic acid sequence with at least about 70% sequence identity to SEQ ID NO: 180, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 180, including all subranges and values that lie therebetween.

In some embodiments, the strain of Veillonella atypica comprises a 16S rRNA gene comprising a nucleic acid sequence comprising at least about 70% sequence identity to SEQ ID NO: 161, for example, at least about 70%, at least about 75%, at least about 80%, at least about 85%, at least about 90%, at least about 95%, at least about 96%, at least about 97.0%, at least about 97.1%, at least about 97.2%, at least about 97.3%, at least about 97.4%, at least about 97.5%, at least about 97.6%, at least about 97.7%, at least about 97.8%, at least about 97.9%, at least about 98.0%, at least about 98.1%, at least 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, at least about 99.9%, or about 100% sequence identity to SEQ ID NO: 161, including all subranges and values that lie therebetween.

Also, the disclosure provides an isolated and purified Veillonella atypica having deposit accession number PTA-126860, or a strain having all of the identifying characteristics of Veillonella atypica PTA-126860, or a mutant thereof.

### (d) Compositions Comprising Athlete-Associated Gut Microbes Disclosed Herein

The disclosure provides compositions comprising any one or more of the strains of Lactobacillus sp. disclosed herein. The disclosure provides compositions comprising any one or more of the strains of Lactobacillus plantarum disclosed herein. The disclosure also provides compositions comprising any one or more of the strains of Lactobacillus rhamnosus disclosed herein. The disclosure also provides compositions comprising any one or more of the strains of Lactobacillus acidophilus disclosed herein. The disclosure also provides compositions comprising any one or more of the strains of Lactobacillus paracasei disclosed herein.

The disclosure provides compositions comprising any one or more of the strains of Bifidobacterium sp. disclosed herein. The disclosure provides compositions comprising any one or more of the strains of Bifidobacterium longum disclosed herein. The disclosure also provides compositions comprising any one or more of the strains of Bifidobacterium animalis lactis disclosed herein.

The disclosure further provides compositions comprising any one or more of the strains of Veillonella sp. disclosed herein. The disclosure provides compositions comprising any one or more of the strains of Veillonella dispar disclosed herein. The disclosure also provides compositions comprising any one or more of the strains of Veillonella parvula disclosed herein. The disclosure also provides compositions comprising any one or more of the strains of Veillonella atypica disclosed herein.

In some embodiments, the amount or concentration of a microbe in the composition is a non-naturally occurring amount or concentration of the microbe. In some embodiments, the composition comprises a non-naturally occurring amount or concentration of each of the microbes in the composition. In some embodiments, the total amount or concentration of all the microbes in the composition is not found in nature. In some embodiments, the composition comprises a non-naturally occurring ratio of the different microbes in the composition.

In some embodiments, the microbes are found in a composition along with an ingredient, carrier, or other component that does not naturally occur with said microbes in their natural state. In embodiments, the edible compositions comprise microbes in combination with a food ingredient that is not found with said microbes in their natural state. In aspects, the compositions herein have markedly different characteristics compared to the microbes found in isolation in nature. In aspects, the microbial compositions are synergistic. In aspects, the disclosure teaches kits, which comprise microbes of the disclosure, along with a suitable packaging that protects the microbes from external stressors. In aspects, kits of the disclosure may comprise directions for use and/or administration of the packaged microbes. Thus, the disclosure teaches packaged microbes and instructions for administration.

In some embodiments, the composition comprises a lactate-producing microbe. The lactate-producing microbe may be any microbe that is capable of producing lactate and/or lactic acid, such as, lactate-producing bacteria. Non-limiting examples of lactate-producing bacteria include bacteria belonging to the genus Lactobacillus, Bifidobacterium, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella. In some embodiments, the lactate-producing bacteria belongs to the genus Lactobacillus. In some embodiments, the lactate-producing bacteria is Lactobacillus plantarum, Lactobacillus acidophilus, Lactobacillus rhamnosus, Lactobacillus paracasei, or any combination thereof. In some embodiments, the lactate-producing bacteria belongs to the genus Bifidobacterium. In some embodiments, the lactate-producing bacteria is Bifidobacterium longum, Bifidobacterium lactis, or a combination thereof. Bifidobacterium lactis may be interchangeably referred to as, Bifidobacterium animalis lactis or Bifidobacterium animalis subsp. lactis. In some embodiments, the composition comprises equal amounts of the Veillonella sp. strain, and one or more of the lactate-producing bacteria. In some embodiments, all the constituent bacteria in the composition are present in a 1:1 ratio.

In some embodiments, the compositions comprise the Veillonella atypica having deposit accession number PTA-126860, or a strain having all of the identifying characteristics of Veillonella atypica PTA-126860, or a mutant thereof, and any one or more of the following microbes: Lactobacillus plantarum, Lactobacillus acidophilus, Lactobacillus rhamnosus, Lactobacillus paracasei, Bifidobacterium longum, and Bifidobacterium lactis. In some embodiments, the compositions comprise the Veillonella atypica having deposit accession number PTA-126860 and Lactobacillus plantarum. In some embodiments, the compositions comprise the Veillonella atypica having deposit accession number PTA-126860 and Lactobacillus acidophilus. In some embodiments, the compositions comprise the Veillonella atypica having deposit accession number PTA-126860 and Lactobacillus rhamnosus. In some embodiments, the compositions comprise the Veillonella atypica having deposit accession number PTA-126860 and Lactobacillus paracasei. In some embodiments, the compositions comprise the Veillonella atypica having deposit accession number PTA-126860 and Bifidobacterium longum. In some embodiments, the compositions comprise the Veillonella atypica having deposit accession number PTA-126860 and Bifidobacterium lactis.

In some embodiments, the compositions comprise the Veillonella parvula having deposit accession number PTA-126859, or a strain having all of the identifying characteristics of Veillonella parvula PTA-126859, or a mutant thereof, and any one or more of the following microbes: Lactobacillus plantarum, Lactobacillus acidophilus, Lactobacillus rhamnosus, Lactobacillus paracasei, Bifidobacterium longum, and Bifidobacterium lactis. In some embodiments, the compositions comprise the Veillonella parvula having deposit accession number PTA-126859 and Lactobacillus plantarum. In some embodiments, the compositions comprise the Veillonella parvula having deposit accession number PTA-126859 and Lactobacillus acidophilus. In some embodiments, the compositions comprise the Veillonella parvula having deposit accession number PTA-126859 and Lactobacillus rhamnosus. In some embodiments, the compositions comprise the Veillonella parvula having deposit accession number PTA-126859 and Lactobacillus paracasei. In some embodiments, the compositions comprise the Veillonella parvula having deposit accession number PTA-126859 and Bifidobacterium longum. In some embodiments, the compositions comprise the Veillonella parvula having deposit accession number PTA-126859 and Bifidobacterium lactis.

In some embodiments, the compositions comprise the Veillonella dispar having deposit accession number PTA-126861, or a strain having all of the identifying characteristics of Veillonella dispar PTA-126861, or a mutant thereof, and any one or more of the following microbes: Lactobacillus plantarum, Lactobacillus acidophilus, Lactobacillus rhamnosus, Lactobacillus paracasei, Bifidobacterium longum, and Bifidobacterium lactis. In some embodiments, the compositions comprise the Veillonella dispar having deposit accession number PTA-126861 and Lactobacillus plantarum. In some embodiments, the compositions comprise the Veillonella dispar having deposit accession number PTA-126861 and Lactobacillus acidophilus. In some embodiments, the compositions comprise the Veillonella dispar having deposit accession number PTA-126861 and Lactobacillus rhamnosus. In some embodiments, the compositions comprise the Veillonella dispar having deposit accession number PTA-126861 and Lactobacillus paracasei. In some embodiments, the compositions comprise the Veillonella dispar having deposit accession number PTA-126861 and Bifidobacterium longum. In some embodiments, the compositions comprise the Veillonella dispar having deposit accession number PTA-126861 and Bifidobacterium lactis.

The disclosure provides compositions comprising: any one or more of the Veillonella sp. strains disclosed herein, and any one or more of the following strains: (a) an isolated and purified Lactobacillus plantarum comprising a 16S rRNA nucleic acid sequence that is at least 97% identical to SEQ ID NO: 1; (b) an isolated and purified Lactobacillus rhamnosus comprising a 16S rRNA nucleic acid sequence that is at least 97% identical to SEQ ID NO: 21; (c) an isolated and purified Lactobacillus acidophilus comprising a 16S rRNA nucleic acid sequence that is at least 97% identical to SEQ ID NO: 41; (d) an isolated and purified Lactobacillus paracasei comprising a 16S rRNA nucleic acid sequence that is at least 97% identical to SEQ ID NO: 61 or SEQ ID NO: 181; (e) an isolated and purified Bifidobacterium longum comprising a 16S rRNA nucleic acid sequence that is at least 97% identical to SEQ ID NO: 81, SEQ ID NO: 201, or SEQ ID NO: 221; and (f) an isolated and purified Bifidobacterium lactis comprising a 16S rRNA nucleic acid sequence that is at least 97% identical to SEQ ID NO: 101.

For instance, in some embodiments, the compositions comprise: (a) an isolated and purified Veillonella atypica having deposit accession number PTA-126860, or a strain having all of the identifying characteristics of Veillonella atypica PTA-126860, or a mutant thereof; (b) an isolated and purified Lactobacillus plantarum comprising a 16S rRNA nucleic acid sequence that is at least 97% identical to SEQ ID NO: 1; (c) an isolated and purified Lactobacillus rhamnosus comprising a 16S rRNA nucleic acid sequence that is at least 97% identical to SEQ ID NO: 21; (d) an isolated and purified Lactobacillus acidophilus comprising a 16S rRNA nucleic acid sequence that is at least 97% identical to SEQ ID NO: 41; (e) an isolated and purified Lactobacillus paracasei comprising a 16S rRNA nucleic acid sequence that is at least 97% identical to SEQ ID NO: 61 or SEQ ID NO: 181; (f) an isolated and purified Bifidobacterium longum comprising a 16S rRNA nucleic acid sequence that is at least 97% identical to SEQ ID NO: 81, SEQ ID NO: 201, or SEQ ID NO: 221; and/or (g) an isolated and purified Bifidobacterium lactis comprising a 16S rRNA nucleic acid sequence that is at least 97% identical to SEQ ID NO: 101.

Without being bound by any one theory, it is thought that in compositions comprising any one or more of the Veillonella sp. disclosed herein, and a lactate-producing microbe (e.g., the Lactobacillus sp. or Bifidobacterium sp.), the lactate produced by the lactate-producing microbe is utilized by the athlete-associated gut microbe to produce acetate and/or propionate. Thus, in some embodiments, the compositions comprising any one or more of the Veillonella sp. disclosed herein, and a lactate-producing microbe (e.g., Lactobacillus sp. or Bifidobacterium sp.) are capable of producing more short chain fatty acids (SCFAs), such as, acetate than the athlete-associated gut microbe alone. In some embodiments, the increased acetate produced by the composition is due to a synergy between the Veillonella sp. and the lactate-producing microbe (e.g., Lactobacillus sp. or Bifidobacterium sp.). In some embodiments, the lactate-producing microbe (e.g., Lactobacillus sp. or Bifidobacterium sp.) does not produce propionate and/or acetate.

In some embodiments, the compositions comprising any one or more of the Veillonella sp. disclosed herein, and a lactate-producing microbe (e.g., Lactobacillus sp. or Bifidobacterium sp.) are capable of producing at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, or about 20 fold, including all subranges and values that lie therebetween) more acetate than the Veillonella sp. alone.

In some embodiments, the compositions comprising any one or more of the Veillonella sp. disclosed herein, and a lactate-producing microbe (e.g., Lactobacillus sp. or Bifidobacterium sp.) are capable of producing at least about 1% (for example, about 2%, about 5%, about 10%, about 15%, about 20%, about 25%, about 30%, about 40%, about 50%, about 60%, about 70%, about 80%, about 90%, or about 100%, including all subranges and values that lie therebetween) more acetate than the Veillonella sp. alone.

In some embodiments, the compositions comprising any one or more of the Veillonella sp. disclosed herein, and a lactate-producing microbe (e.g., Lactobacillus sp. or Bifidobacterium sp.) produce less propionate than the Veillonella sp. alone. In some embodiments, the compositions comprising any one or more of the Veillonella sp. disclosed herein, and a lactate-producing microbe (e.g., Lactobacillus sp. or Bifidobacterium sp.) produce at least about 1% (for example, about 2%, about 5%, about 10%, about 15%, about 20%, about 25%, about 30%, about 40%, about 50%, about 60%, about 70%, about 80%, about 90%, or about 100%, including all subranges and values that lie therebetween) less propionate than the Veillonella sp. alone.

In some embodiments, the compositions comprising any one or more of the Veillonella sp. disclosed herein, and a lactate-producing microbe (e.g., Lactobacillus sp. or Bifidobacterium sp.) are capable of producing more acetate and less propionate than the Veillonella sp. alone.

In some embodiments, the compositions disclosed herein comprise an additive that is capable of enhancing one or more functional properties of the microbial strains comprised in the composition. For instance, the additive may increase the level of short chain fatty acids (SCFAs), such as, acetate and/or propionate, produced by the Veillonella strain. In some embodiments, the additive that is capable of increasing the level of acetate and/or propionate produced by the Veillonella strain is a lactate. In some embodiments, the lactate is lactate ion or lactic acid. In some embodiments, the lactate is sodium lactate, magnesium lactate, calcium lactate, manganese lactate, potassium lactate, cobalt lactate or iron lactate. Without being bound by a theory, it is thought that Veillonella is capable of metabolizing lactates, which in turn might contribute to an increase in acetate and/or propionate production. Further, lactate salts, such as, sodium lactate, magnesium lactate, calcium lactate, and potassium lactate can also act as electrolytes and promote body hydration. In some embodiments, the additive is a nitrate, such as sodium nitrate. Without being bound by any one theory, it is thought that the Veillonella strain metabolizes nitrate to produce nitric oxide, which also has beneficial properties in increasing endurance.

In some embodiments, the compositions comprise one or more additives, such as, dietary supplements. In some embodiments, the dietary supplement is a probiotic or prebiotic known to one of skill in the art. In some embodiments, the dietary supplement is one or more non-digestible dietary supplements, which modifies the balance of the intestinal micro flora, stimulates the growth and/or activity of beneficial microorganisms and suppresses potentially deleterious microorganisms. Non-limiting examples of dietary supplements include oligosaccharides (fructo-oligosaccharides, galacto-oligosaccharides), inulin, lactulose, lactitol and select bacterial strains that produce nutrients that promote the growth of beneficial bacteria, such as within the intestinal tract. In some embodiments, the dietary supplement is a dietary fiber, such as, for example, cellulose, chitin, hemicellulose, lignin, xanthan gum, fructans, pectin, alginates (e.g., sodium alginate), agar, carrageen, raffinose and polydextrose. In some embodiments, the dietary supplement is an amino acid, or a protein.

Any one of the compositions disclosed herein may further comprise any conventional food supplement fillers and extenders such as, for example, a flour, a binder, a nutraceutical compound or formulation, or a nutrient such as, an amino acid (e.g., branched chain amino acids, such as, leucine, isoleucine, and valine), a vitamin (e.g., vitamin D, folate, vitamin B-12), or a mineral (e.g., magnesium, calcium, zinc, iron). In some embodiments, the additive is an anti-oxidant (e.g., vitamin C, vitamin E, beta-carotene, carotenoids, selenium, manganese, lycopene, lutein and zeaxanthin), an anti-inflammatory substance (e.g., alpha-lipoic acid, curcumin, fish oil, ginger, resveratrol, spirulina) and/or an adaptogen (e.g., ginseng). In some embodiments, the additive is maltodextrin.

In some embodiments, the additive is capable of enhancing exercise endurance in a subject when administered to the subject. Non-limiting examples of such additives include caffeine, beta-alanine, carnosine, anserine, carbonate, carbohydrates, electrolytes, glycerol, nitrates, citrulline, and arginine. In some embodiments, the additive is capable of improving the athletic performance of a subject when administered to the subject. Non-limiting examples of such additives include creatine, protein, amino acids, essential amino acids (EAA), branched chain amino acids, beta-hydroxy beta-methylbutyric acid (HMB), 2-Hydroxyisocaproic acid (HICA), adenosine 5′-triphosphate (ATP), phosphatidic acid, phosphatidylserine, arachidonic acid, taurine, carnitine, and medium chain fatty acids. In some embodiments, the additive is capable of reducing inflammation in a subject when administered to the subject. Non-limiting examples of such additives include curcumin and omega-3 fatty acids. In some embodiments, the additive is capable of enhancing recovery from physical exercise in a subject when administered to the subject. Non-limiting examples of such additives include β-glucan, HMB, BCAAs, protein, curcumin, anti-oxidants, such as vitamin E, CoQ10, and alpha-lipoic acid.

In some embodiments, the additive is a vegetable extract or a fruit extract. In some embodiments, the fruit extract or vegetable extract naturally contains lactic acid. In some embodiments, the fruit extract is derived from Akebia fruit.

In some embodiments, the composition is a human edible formulation. In some embodiments, the composition is in a solid form (such as a lyophilized powder suitable for reconstitution), a liquid solution, suspension, emulsion, tablet, caplet, syrup, gummies, gels, pill, capsule, sustained release formulation, or powder. In some embodiments, the composition is a dry powder. In some embodiments, the composition is an encapsulated powder. In some embodiments, the composition is designed such that the microbe is not in contact with moisture and/or air. In some embodiments, delivery vehicles such as liposomes, nanocapsules, microparticles, microspheres, lipid particles, vesicles, and the like, may be used for the delivery of the microbial strains or compositions disclosed herein. In some embodiments, the composition is a human edible formulation (e.g., an organic human edible formulation), such as, for example, a protein powder, a protein supplement, a protein supplement shake, a protein supplement formed and packed as a bar, a beverage, a gel composition, a food composition, a freeze dried product or powder, a cereal bar, an energy bar (e.g., nutritional supplement energy bar, an organic energy bar), a fruit-based food bar (e.g., an organic fruit-based food bar), a fruit-based meal replacement bar, a nut-based food bar, a nut-based snack bar, a yogurt, a yoghurt drink, a yoghurt-based beverage, a kefir, a electrolyte replacement solution, a sports drink (e.g., sports drinks containing electrolytes, vitamins and probiotics), a non-alcoholic water-based beverage (e.g., one containing electrolytes, vitamins and probiotics); a protein-enriched sports beverage, a non-alcoholic water-based beverage (e.g., one containing protein), a caplet, a chewable formulation, granule, a dissolvable formulation, and/or fermented, dairy product, such as a chilled dairy product, or a fresh cheese. For instance, in some embodiments, a dry powdered form of the microbe or composition disclosed herein may be spread over a cereal bar, an energy bar, or any human edible formulation described herein. In some embodiments, an encapsulated form of the microbe or composition disclosed herein may be included in a liquid edible formulation, such as, a beverage. In some embodiments, the composition is a probiotic composition for use as ingredient for food or beverage, such as probiotic bacteria or probiotic bacterial cultures. In some embodiments, the composition is a probiotic supplement or nutritional supplement, or used in the manufacture of a probiotic supplement or nutritional supplement. In some embodiments, the composition is in the form of a beverage containing probiotics for use as a nutritional supplement. In some embodiments, the composition is a nutritional probiotic supplement in the form of capsules, tablets, caplets, powder, syrups, gummies and gels.

Any one of the compositions disclosed herein may further comprise a pharmaceutically acceptable carrier. By “pharmaceutically acceptable” it is meant a material that is not toxic or otherwise undesirable, i.e., the material may be administered to a subject without causing any significant undesirable biological effects. Pharmaceutically acceptable carriers or excipients are described, for example, in Remington's Pharmaceutical Sciences, Mack Publishing Co. (A R. Gennaro edit. 1985), Handbook of Pharmaceutical Additives, 2nd Edition (eds. M. Ash and I. Ash), 2001 (Synapse Information Resources, Inc., Endicott, N.Y., USA), and Handbook of Pharmaceutical Excipients, 2nd edition, 1994, the contents of each of which is herein incorporated by reference in its entirety for all purposes. In some embodiments, the compositions disclosed herein further comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

Any one of the compositions disclosed herein may further comprise a food carrier, or an edible carrier. As used herein, a “food carrier” or an “edible carrier” refer to materials that may be present in human edible formulations, such as food and beverages, and that are not toxic or otherwise undesirable, i.e., the food carrier and/or edible carrier may be consumed by a subject without resulting in any significant undesirable biological effects. Non-limiting examples of food carriers and/or edible carriers include, food acids (e.g. vinegar, citric acid, tartaric acid, malic acid), acidity regulators, anticaking agents, antifoaming agents, bulking agents, food coloring agents, color retention agents, emulsifiers, flavors, flavor enhancers, antioxidants, glazing agents, humectants, tracer gases, preservatives, stabilizers, sweeteners, and thickeners.

In some embodiments, the compositions disclosed herein further comprise one or more additives that promotes the growth of the one or more microbial strains in the composition and/or increases the ability of the microbial strain to be active in the gut. Non-limiting examples of such additives include cellobiose, maltose, mannose, salicine, trehalose, amygdalin, arabinose, melobiose, rhamnose and/or xylose. In some embodiments, the composition further comprises a drug, such as, acetaminophen, antibiotics such as an antibiotic selected from the group consisting of vancomycin, metronidazole, gentamicin, colistin, fidaxomicin, telavancin, oritavancin, dalbavancin and daptomycin, or any other beneficial substances. In some embodiments, the compositions disclosed herein further comprise pH encapsulated glucose, lipids or proteins, which are released at a pH in the range of about 1 to about 6. In some embodiments, the pH encapsulated glucose, lipids or proteins are released before the release of the one or more microbial strains in the composition. In some embodiments, the compositions disclosed herein enable the release of the athlete-associated gut microbes in the distal segments of the gastrointestinal tract, including the ileum and colon of the subject.

### Methods of Producing Compositions Comprising Athlete-Associated Microbial Strains

The disclosure provides methods of identifying and characterizing athlete-associated gut microbial strains. In some embodiments, the methods of identifying athlete-associated gut microbial strains, comprise isolating a stool sample of the athlete, and identifying the microbial strains that are present in the stool sample. In some embodiments, the identification step comprises isolating the DNA of microbial strains present in the stool sample, sequencing parts or whole of the 16S ribosomal DNA (rDNA) gene of the microbial strains present in the stool samples, and identifying the microbial strains present based on sequence alignment techniques.

The methods involved in the identification of microbes present in fecal sample, such as sequencing and sequence alignment, and subsequent characterization of the microbes are further described in “Probiotic formulations for improving athletic performance” WO 2017/180501 A1, published Oct. 19, 2017; Scheiman et al., “Meta-omics analysis of elite athletes identifies a performance-enhancing microbe that functions via lactate metabolism” Nature Medicine volume 25, Jul. 2019, pages 1104-1109; and “Compositions and methods for enhancing exercise endurance” WO 2020/172604 A1, published Aug. 27, 2020, the contents of each of which is incorporated by reference herein in its entireties for all purposes.

Scheiman et al. observed that bacteria of the genus Veillonella are enriched in some athletes after exercise, and they isolated V. parvula, V. dispar and V. atypica from these athletes. They also saw that when V. atypica is inoculated into mice, exhaustive treadmill run time of the mice is increased, indicating that V. atypica can enhance physical performance. They also describe that Veillonella methylmalonyl-CoA pathway is overrepresented in athletes after exercise. Based on these results, Scheiman et al. concluded that bacteria of the genus Veillonella, such as V. atypica, can enhance athletic performance by increasing the levels of one or more performance-enhancing molecules, such as acetate and propionate, and potentially also by reducing the levels of lactate/lactic acid in the subj ect.

The present disclosure is aimed at finding additional, specific strains of V. parvula, V. dispar and V. atypica, which are genetically distinct from those of Scheiman et al., based on, for example, their 16S rRNA sequences. The disclosed strains, which were isolated from different athletes, are hypothesized to also be beneficial for improving athletic performance, reducing inflammatory cytokines, and enhancing endurance.

In some embodiments, the methods further comprise identifying microbial strains that are associated with the gut of a control human subject (such as, a non-athlete), comprising isolating a stool sample of the control subject, and identifying the microbial strains that are present in the stool sample, using the methods described above. In some embodiments, the methods further comprise comparing the diversity, proportion, and/or population of the gut microbial strains identified in an athlete with the diversity, proportion, and/or population of the gut microbial strains identified in a non-athlete.

In some embodiments, the methods further comprise comparing the lactate or lactic acid metabolizing activity of the gut microbial strains identified in an athlete with the lactate or lactic acid metabolizing activity of the gut microbial strains identified in a non-athlete. In some embodiments, the methods further comprise comparing the propionate or propionic acid producing activity of the gut microbial strains identified in an athlete with the propionate or propionic acid producing activity of the gut microbial strains identified in a non-athlete. The metabolic activity of the microbial strains, for example the lactate or lactic acid metabolizing activity, or the propionate, propionic acid, acetic acid and/or acetate-producing activity, may be determined using approaches, such as mass spectrometry (MS) of the media used to culture the microbial strains.

The athlete-associated microbial strains disclosed herein may be grown for commercial purposes using methods described herein. Any suitable medium, such as medium comprising a carbon-based substrate and/or a carbon based energy source may be used. In some embodiments, the media comprises lactic acid and/or lactates. In some embodiments, the strains may be grown under conditions of anaerobiosis. In some embodiments, the athlete-associated microbial strains disclosed herein are live strains. Without being bound by a theory, it is thought that the ingestion of live athlete-associated microbial strains may enable growth and colonization of the gut tissue in the small and large intestines. The microbial cells may be recovered by centrifugation. Centrifugation may performed, for example at a speed in the range of about 10,000 g to about 15,000 g, such as 12,000 g, for about 15 to about 20 minutes. In some embodiments, the microbial cells may be washed in, for example, an anaerobic phosphate buffer, by resuspension of the cells, agitation, and a further centrifugation step.

In some embodiments, the athlete-associated microbial strains disclosed herein may be processed for commercial purposes using methods, such as fermentation, drying and pulverization. Details of these methods are provided in EP0818529 and WO2001/044440, the contents of each of which are herein incorporated by reference in their entireties for all purposes. In some embodiments, the athlete-associated microbial strains may be concentrated from a medium and dried by spray drying, fluidized bed drying, lyophilization (freeze drying) or other drying processes. In some embodiments, the athlete-associated microbial strains may be mixed with a carrier material such as a carbohydrate such as sucrose, lactose or maltodextrin, a lipid or a protein, for example milk powder during or before the drying.

In some embodiments, the athlete-associated microbial strains disclosed herein may be mixed with a food product directly after fermentation. In some embodiments, a drying process is performed thereafter. Details of these methods are provided in EP0818529 and PCT/EP02/01504, the contents of each of which are herein incorporated by reference in their entireties for all purposes. In some embodiments, the athlete-associated microbial strains disclosed herein may be dried with a food product as described in WO1998/10666, which is incorporated herein by reference in its entirety for all purposes. In some embodiments, the athlete-associated microbial strains disclosed herein may be dried with juices, milk-based products or vegetable milks. Such a product may later be reconstituted with an aqueous liquid. In some embodiments, the athlete-associated microbial strains and/or compositions disclosed herein may be added to a food product, such as a nutritional formula, breakfast cereals, salads, or a slice of bread, prior to consumption.

In some embodiments, the athlete-associated microbial strains disclosed herein may be microencapsulated. Without being bound to a theory, it is thought that microencapsulation formulations and techniques protect the microbes disclosed herein from the digestive actions of the stomach, duodenum, and jejunum of the intestine, and allow administration, delivery or release to the gut or ileum of the subject. Microencapsulated microbes may be co-administered with drugs, foods, nutrients, vitamins, other beneficial substances, prebiotics, and other therapeutic agents such as pH encapsulated glucose, lipids or proteins that release in the distal small intestine at pH values between 7.0 and 8.0. Preferably, at least two coatings are used to cover a tablet or capsule like form comprising the athlete-associated gut microbe, wherein the outside coating is degraded in a pH environment of 5 to 6 and the inside coating is degraded in a pH environment of about 7 thereby releasing the athlete-associated gut microbe in the ileum area and in close proximity to the Peyer's Patches.

An exemplary coating may include one or more of poly(dl-lactide-co-glycolide, chitosan, casein, chitosan (Chi) stabilized with PVA (poly-vinylic alcohol), a lipid, an alginate, carboxymethylethylcellulose (CMEC), cellulose acetate trimellitiate (CAT), hydroxypropylmethyl cellulose phthalate (HPMCP), hydroxypropylmethyl cellulose, ethyl cellulose, color con, food glaze and mixtures of hydroxypropylmethyl cellulose and ethyl cellulose, polyvinyl acetate phthalate (PV AP), cellulose acetate phthalate (CAP), shellac, copolymers of methacrylic acid and ethyl acrylate, and copolymers of methacrylic acid and ethyl acrylate to which a monomer of methylacrylate has been added during polymerization.

The processing steps for producing the athlete-associated microbial strain and/or composition may be used in any order or combination, and any processing step may be omitted or included as determined by a person of skill in the art.

The disclosure further provides a kit including one or more of the athlete-associated microbial strains and/or compositions disclosed herein. In some embodiments, one or more of the athlete-associated microbial strains and/or compositions disclosed herein may be provided within a container. In some embodiments, the kit further comprises one or more devices for measuring units of the athlete-associated microbial strains and/or compositions for administration to the subject, and instructions for administration.

### Methods of Using the Athlete-Associated Microbial Strains and Compositions Disclosed Herein

The disclosure provides methods of using any one or more of the athlete-associated microbial strains and/or compositions disclosed herein, comprising administering the athlete-associated microbial strains and compositions to the subject. Without being bound to a theory, it is thought that after administration, the microbial strains disclosed herein may adhere to the gut tissue of the subject, such as the wall of the intestine or other gut tissue and multiply, thereby altering the gut microbiome. The disclosure thus provides methods of altering the microbiome of a subject, comprising administering to the subject an effective dose of any one or more of the athlete-associated microbial strains and/or compositions disclosed herein. For instance, in some embodiments, the administration results in increasing the population (or number or abundance) of the one or more athlete-associated gut microbes in the subject. In some embodiments, the administration results in increasing the proportion of the one or more athlete-associated gut microbes in the subject. In some embodiments, the administration results in an increase in the population of the one or more athlete-associated gut microbes in the subject by a value in the range of about 1% to about 1000%, for example about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1000%, including all subranges and values that lie therebetween.

In some embodiments, the methods comprise an increase in healthy gut microflora in the subject after the administration of the one or more athlete-associated microbes and/or compositions disclosed herein, as compared to before the administration of the one or more athlete-associated microbes and/or compositions disclosed herein. In some embodiments, the healthy gut microflora in the subject is increased by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein.

In some embodiments, the methods comprise a decrease in pathogenic gut microflora in the subject after the administration of the one or more athlete-associated microbes and/or compositions disclosed herein, as compared to before the administration of the one or more athlete-associated microbes and/or compositions disclosed herein. In some embodiments, the pathogenic gut microflora in the subject is decreased by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein.

In some embodiments, the athlete-associated gut microbe, or composition disclosed herein is capable of metabolizing lactic acid and/or lactates. Without being bound to a theory, it is thought that the administration of the one or more athlete-associated gut microbes, or compositions disclosed herein capable of metabolizing lactic acid and/or lactates may be associated with a decrease in the level of lactic acid and/or lactates in the subject. Therefore, in some embodiments, the level of lactic acid and/or lactates in the subject is lower after the administration of the one or more athlete-associated gut microbes and/or compositions disclosed herein, as compared to before the administration of the one or more athlete-associated gut microbes and/or compositions disclosed herein. Without being bound by theory, it is thought that lower levels of lactic acid and/or lactates in the blood correlate with higher exercise endurance and/or enhanced recovery from physical exercise.

In some embodiments, the level of lactic acid and/or lactates in the subject is lower by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein. The level of lactic acid and/or lactates may refer the level of lactic acid in the blood, serum, gut, or any other organ, tissue or cell of the subject. In some embodiments, the level of lactic acid and/or lactates is the systemic level of lactic acid and/or lactates.

In some embodiments, the athlete-associated gut microbe is capable of producing propionic acid and/or propionates. Without being bound to a theory, it is thought that the administration of the one or more athlete-associated gut microbes disclosed herein capable of producing propionic acid and/or propionates may be associated with an increase in the level of propionic acid and/or propionates in the subject. In some embodiments, the level of propionic acid or propionates in the subject is higher after the administration of the one or more athlete-associated gut microbes and/or compositions disclosed herein, as compared to before the administration of the one or more athlete-associated gut microbes and/or compositions disclosed herein. Without being bound by theory, it is thought that higher levels of propionic acid and/or propionates in the blood correlate with higher exercise endurance, enhanced recovery from physical exercise, higher heart rate, maximum rate of oxygen consumption, higher energy expenditure, and/or higher lipid oxidation.

In some embodiments, the level of propionic acid or propionates in the subject is higher by about 1% to about 1000%, for example, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1000%, including all subranges and values that lie therebetween, after the administration of the one or more athlete-associated gut microbes and/or compositions disclosed herein, as compared to before the administration of the one or more athlete-associated gut microbes and/or compositions disclosed herein. The level of propionic acid and/or propionates may refer the level of propionic acid or propionates in the blood, serum, gut, or any other organ, tissue or cell of the subject. In some embodiments, the level of propionic acid or propionates is the systemic level of propionic acid or propionates.

In some embodiments, the athlete-associated gut microbe is capable of producing acetic acid and/or acetates. Without being bound to a theory, it is thought that the administration of the one or more athlete-associated gut microbes disclosed herein capable of producing acetic acid and/or acetates may be associated with an increase in the level of acetic acid and/or acetates in the subject. In some embodiments, the level of acetic acid and/or acetates in the subject is higher after the administration of the one or more athlete-associated gut microbes and/or compositions disclosed herein, as compared to before the administration of the one or more athlete-associated gut microbes and/or compositions disclosed herein. Without being bound by theory, it is thought that higher levels of acetic acid and/or acetates in the blood correlate with higher exercise endurance, enhanced recovery from physical exercise, higher heart rate, maximum rate of oxygen consumption, higher energy expenditure, higher lipid oxidation, and/or enhanced anti-inflammatory effects.

In some embodiments, the level of acetic acid and/or acetates in the subject is higher by about 1% to about 1000%, for example, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1000%, including all subranges and values that lie therebetween, after the administration of the one or more athlete-associated gut microbes and/or compositions disclosed herein, as compared to before the administration of the one or more athlete-associated gut microbes and/or compositions disclosed herein. The level of acetic acid and/or acetates may refer the level of acetic acid and/or acetates in the blood, serum, gut, or any other organ, tissue or cell of the subject. In some embodiments, the level of acetic acid and/or acetates is the systemic level of acetic acid and/or acetates.

In some embodiments, the administration of the one or more compositions disclosed herein (such as, a composition comprising one or more of the athlete-associated gut microbes disclosed herein, and one or more lactate producing bacteria) is associated with a larger increase in the level of SCFAs, such as acetic acid and/or acetates in the subject, as compared to administration of the one or more of the athlete-associated gut microbes alone. That is, the level of acetic acid and/or acetates in the subject after the administration of the one or more compositions (such as, a composition comprising one or more of the athlete-associated gut microbes disclosed herein, and one or more lactate producing bacteria) disclosed herein is higher, as compared to after the administration of the one or more of the athlete-associated gut microbes alone.

In some embodiments, the level of SCFAs, such as, acetic acid and/or acetates in the subject is higher by about 1% to about 100%, for example, about 5%, about 10%, about 20%, about 30%, about 40%, about 50%, about 60%, about 70%, about 80%, about 90%, about 100%, including all subranges and values that lie therebetween, after the administration of a composition comprising one or more of the Veillonella sp. disclosed herein, and one or more lactate producing bacteria (e.g., Lactobacillus sp. or Bifidobacterium sp.), as compared to after the administration of the one or more Veillonella sp. alone.

In some embodiments, the administration of the one or more compositions disclosed herein (such as, a composition comprising one or more of the athlete-associated gut microbes disclosed herein, and one or more lactate producing bacteria) is associated with a decrease in the level of propionic acid and/or propionates in the subject, as compared to administration of the athlete-associated gut microbe alone. That is, the level of propionic acid and/or propionates in the subject after the administration of the one or more compositions (such as, a composition comprising one or more of the Veillonella sp. disclosed herein, and one or more lactate producing bacteria) disclosed herein is lower, as compared to after the administration of the one or more Veillonella sp. alone.

In some embodiments, the level of propionic acid and/or propionates in the subject is lower by about 1% to about 100%, for example, about 5%, about 10%, about 20%, about 30%, about 40%, about 50%, about 60%, about 70%, about 80%, about 90%, about 100%, including all subranges and values that lie therebetween, after the administration of a composition comprising one or more of the Veillonella sp. disclosed herein, and one or more lactate producing bacteria (e.g., Lactobacillus sp. or Bifidobacterium sp.), as compared to after the administration of the one or more Veillonella sp. alone.

Scheiman et al., Nature Medicine volume 25, pages1104-1109 (2019) observed that the administration of propionate improved exercise endurance and athletic performance, but did not impact inflammatory cytokines. Acetate, on the other hand, has been shown to improve endurance and have anti-inflammatory properties. Without being bound to any one theory, it is thought that administration of a composition, comprising a Veillonella strain disclosed herein and a lactate producing bacteria (e.g., Lactobacillus sp. or Bifidobacterium sp.) to a subject may be beneficial to the subject due to increased production of acetates, which has endurance-improving and anti-inflammatory properties.

In some embodiments, the subject undertakes one or more physical exercise before, during, or after the administration of the one or more athlete-associated gut microbes and/or compositions disclosed herein. In some embodiments, the subject undertakes one or more physical exercise before or after the administration of the one or more athlete-associated gut microbes and/or compositions disclosed herein; wherein the time period between the administration of the one or more athlete-associated gut microbe and undertaking of physical exercise is not limited, and may be, for example, in the range of about 1 minute to about several months, for example, 5 min, 30 min, 1 hour, 6 hours, 12 hours, 24 hours, 2 days, 3 days, 4 days, 5 days, 6 days, 1 week, 2 weeks, 3 weeks, 1 month, 1 year, including all subranges and values that lie therebetween.

In some embodiments, the level of lactic acid and/or lactates in the subject, who is administered one or more athlete-associated microbial strains and/or compositions disclosed herein, is lower after the physical exercise, as compared to a control subject. As used herein, a “control subject” is a subject who undertakes the same physical exercise as the subject, and is otherwise similar to the subject based on physiological factors, but is not administered the one or more athlete-associated gut microbes and/or compositions disclosed herein. In some embodiments, the level of lactic acid and/or lactates in the subject after physical exercise is lower by about 1% to about 100%, for example, about 5%, about 10%, about 20%, about 30%, about 40%, about 50%, about 60%, about 70%, about 80%, about 90%, about 100%, including all subranges and values that lie therebetween, as compared to the level of lactic acid and/or lactates in the control subject after physical exercise.

In some embodiments, the level of propionic acid, propionates, acetic acid, and/or acetates in the subject, who is administered one or more athlete-associated microbial strains and/or compositions disclosed herein, is higher after the physical exercise, as compared to the control subject. In some embodiments, the level of propionic acid, propionates, acetic acid, and/or acetates in the subject after physical exercise is higher by about 1% to about 100%, for example, about 5%, about 10%, about 20%, about 30%, about 40%, about 50%, about 60%, about 70%, about 80%, about 90%, about 100%, including all subranges and values that lie therebetween, as compared to the level of propionic acid, propionates acetic acid, and/or acetates in the control subject after physical exercise.

In some embodiments, the level of SCFAs, such as, acetic acid and/or acetates in the subject, who is administered a composition disclosed herein, comprising one or more of the Veillonella sp. disclosed herein, and one or more lactate producing bacteria (e.g., Lactobacillus sp. or Bifidobacterium sp.), is higher after the physical exercise, as compared to a subject, who is administered one or more of the Veillonella sp. alone. In some embodiments, the level of acetic acid and/or acetates in the subject, who is administered a composition disclosed herein, comprising one or more of the Veillonella sp. disclosed herein, and one or more lactate producing bacteria (e.g., Lactobacillus sp. or Bifidobacterium sp.), is higher after physical exercise by about 1% to about 100%, for example, about 5%, about 10%, about 20%, about 30%, about 40%, about 50%, about 60%, about 70%, about 80%, about 90%, about 100%, including all subranges and values that lie therebetween, as compared to a subject, who is administered one or more of the Veillonella sp. alone.

In some embodiments, the administration of the one or more athlete-associated gut microbes and/or compositions disclosed herein to the subject is associated with, promotes or causes enhanced exercise endurance, improved athletic performance, reduced inflammation, enhanced recovery from physical exercise, a decrease in fatigue, or any combination thereof in the subject. Without being bound by a theory, it is thought that a decrease in the levels of lactic acid and/or lactates, for example in the blood of the subject, and/or an increase in the levels of propionic acid, propionates, acetic acid, and/or acetates for example in the blood of the subject may be associated with, promote or cause enhanced exercise endurance, improved athletic performance, reduced inflammation, enhanced recovery from physical exercise, a decrease in fatigue, or any combination thereof in the subj ect.

Thus, the disclosure provides methods of enhancing exercise endurance of a subject, comprising administering to the subject any one or more of the athlete-associated gut microbes and/or compositions disclosed herein. The disclosure further provides methods of improving the athletic performance of a subject, comprising administering to the subject any one or more of the athlete-associated gut microbes and/or compositions disclosed herein.

In some embodiments, the method comprises an improvement of one or more objective factors related to a physical exercise for the subject, who is administered one or more athlete-associated microbial strains and/or compositions disclosed herein. In some embodiments, the physical exercise is running. Therefore, in some embodiments, the method comprises an improvement of one or more objective factors related to running, such as, for example, the maximum time the subject is able to run until exhaustion point (also called, “maximum run time”), the maximum distance run until exhaustion point (also called, “maximum run distance”), the distance run in a particular time period, and the time taken to run a particular distance. In some embodiments, the maximum run time of the subject who is administered the one or more athlete-associated gut microbes and/or compositions disclosed herein is increased by about 1% to about 100%, for example, about 5%, about 10%, about 20%, about 30%, about 40%, about 50%, about 60%, about 70%, about 80%, about 90%, about 100%, including all subranges and values that lie therebetween, as compared to the control subject. As used herein, a control subject undertakes the same physical exercise as the subject (that is, running), and is otherwise similar to the subject based on physiological factors, but is not administered the one or more athlete-associated gut microbes and/or compositions disclosed herein. In some embodiments, the maximum run distance of the subject who is administered the one or more athlete-associated gut microbes and/or compositions disclosed herein is increased by about 1% to about 100%, for example, about 5%, about 10%, about 20%, about 30%, about 40%, about 50%, about 60%, about 70%, about 80%, about 90%, about 100%, including all subranges and values that lie therebetween, as compared to the control subject. In some embodiments, the distance run in a particular time period of the subject who is administered the one or more athlete-associated gut microbes and/or compositions disclosed herein is increased by about 1% to about 100%, for example, about 5%, about 10%, about 20%, about 30%, about 40%, about 50%, about 60%, about 70%, about 80%, about 90%, about 100%, including all subranges and values that lie therebetween, as compared to the control subject. In some embodiments, the time taken to run a particular distance of the subject who is administered the one or more athlete-associated gut microbes and/or compositions disclosed herein is decreased by about 1% to about 95%, for example, about 5%, about 10%, about 20%, about 30%, about 40%, about 50%, about 60%, about 70%, about 80%, or about 90%, including all subranges and values that lie therebetween, as compared to the control subject.

In some embodiments, the methods comprise an improvement in digestion in the subject after the administration of the one or more athlete-associated microbes and/or compositions disclosed herein, as compared to before the administration of the one or more athlete-associated microbes and/or compositions disclosed herein. In some embodiments, digestion in the subject is increased or improved by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein.

In some embodiments, the methods comprise an improvement in the metabolism of a subject after the administration of the one or more athlete-associated microbes and/or compositions disclosed herein, as compared to before the administration of the one or more athlete-associated microbes and/or compositions disclosed herein. In some embodiments, metabolism in the subject is increased by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein.

In some embodiments, the methods comprise an improvement in the immune system of the subject after the administration of the one or more athlete-associated microbes and/or compositions disclosed herein, as compared to before the administration of the one or more athlete-associated microbes and/or compositions disclosed herein. In some embodiments, immunity in the subject is increased by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein.

In some embodiments, the methods comprise a reduction in the level of at least one inflammatory cytokine in the subject after the administration of the one or more athlete-associate microbes and/or compositions disclosed herein, as compared to before the administration of the one or more athlete-associate microbes and/or compositions disclosed herein. In some embodiments, the level of the at least one inflammatory cytokine in the subject is decreased by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein. Without being bound by a theory, it is thought that lower levels of inflammatory cytokines is correlated with reduced inflammation and/or enhanced recovery from physical exercise. Therefore, the disclosure provides methods of reducing inflammation in a subject in need thereof, comprising administering to the subject any one or more of the athlete-associated gut microbes and/or compositions disclosed herein. The disclosure further provides methods of enhancing recovery from physical exercise in a subject in need thereof, comprising administering to the subject any one or more of the athlete-associated gut microbes and/or compositions disclosed herein.

In some embodiments, the level of the at least one inflammatory cytokine after physical exercise in the subject, who is administered one or more microbial strains and/or composition disclosed herein, is lower by about 1% to about 100%, for example, about 5%, about 10%, about 20%, about 30%, about 40%, about 50%, about 60%, about 70%, about 80%, about 90%, about 100%, including all subranges and values that lie therebetween, as compared to the level of the at least one inflammatory cytokine in the control subject after physical exercise.

The disclosure further provides methods of increasing muscle mass and/or muscular strength of a subject in need thereof, comprising administering to the subject an effective dose of any one or more of the athlete-associated gut microbes and/or compositions disclosed herein. The disclosure further provides methods of preventing the loss of muscle mass in a subject in need thereof, comprising administering to the subject an effective dose of any one or more of the athlete-associated gut microbes and/or compositions disclosed herein. In some embodiments, muscle loss may be age-related muscle loss (sarcopenia), cachexia, or muscle disuse atrophy due to for instance, hospitalization, immobilization, or injury. In some embodiments, muscle loss may be due to intake of calorie-deficient diets.

The effective dosage of the one or more of the athlete-associated microbial strains disclosed herein is not limited, and may be determined by the subject, either alone, or in consultation with a person skilled in the art, physician or athletic trainer. In some embodiments, the dosage enables the successful colonization of the gut tissue. In some embodiments, the dosage may be in range of about 10⁴ colony forming units (CFU) to 10¹⁶ CFU, for example, about 10⁵ CFU, about 10⁶ CFU, about 10⁷ CFU, about 10⁸ CFU, about 10⁹ CFU, about 10¹⁰ CFU, about 10¹¹ CFU, about 10¹² CFU, about 10¹³ CFU, about 10¹⁴ CFU, or about 10¹⁵ CFU, including all subranges and values that lie therebetween. In some embodiments, the dosage is in the range of about 10⁹ CFU to about 10¹¹ CFU. In some embodiments, the dosage in the range of about 5×10⁹ CFU to about 10¹⁰ CFU. In some embodiments, the dosage in the range of about 5×10⁹ CFU to about 15×10⁹ CFU.

The frequency of dosage administered is not limited, and may be determined by a person skilled in the art, either alone, or in consultation with a physician or athletic trainer. In some embodiments, the one or more of the athlete-associated microbial strains may be administered to the subject once a day or more than once a day. In some embodiments, the one or more of the athlete-associated microbial strains may be administered to the subject twice, thrice, four, five, six, 7, 8, 9 or 10 times a day. In some embodiments, the one or more of the athlete-associated microbial strains may be administered to the subject weekly or a few times a week. In some embodiments, the one or more of the athlete-associated microbial strains may be administered to the subject every day, every alternate, every third day, every fourth day, every fifth day, or every sixth day. In some embodiments, the one or more of the athlete-associated microbial strains may be administered to the subject weekly, bi-weekly or every three weeks. In some embodiments, the one or more of the athlete-associated microbial strains may be administered to the subject every 1, 2, 3, 4, 5, 6, 7, 8, 9, 10 or 11 months. In some embodiments, the one or more of the athlete-associated microbial strains may be administered to the subject every year. In some embodiments, the one or more of the athlete-associated microbial strains may be administered to the subject every 1, 2, 3, 4, 5, 10, 15, or 20 years. In some embodiments, the one or more of the athlete-associated microbial strains may be administered to the subject before, during or after performing a physical exercise.

Administration of the one or more of the athlete-associated microbial strains and/or compositions disclosed herein to the subject may be any route that is determined to be effective by a person skilled in the art, a physician or an athletic trainer. In some embodiments, the administration is via oral, enteric, gastrointestinal, rectal, or parenteral route.

The disclosure provides methods of generating model animals, comprising administering or transplanting any one or more of athlete-associated gut microbes and/or compositions disclosed herein into the animal. In some embodiments, the transplantation is done to the gut tissue of the animal. In some embodiments, the model animal is a mouse, for example a germ-free mouse. In some embodiments, the methods further comprise screening the model animals transplanted with any one or more of athlete-associated gut microbes and/or compositions to determine their physiological and physical characteristics.

The disclosure also provides methods of improving the athletic performance of an animal, comprising administering any one or more athlete-associated gut microbes and/or compositions disclosed herein to the animal. In some embodiments, the administration is via oral, enteric, gastrointestinal, or rectal route. In some embodiments, the animal is a horse.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present disclosure includes preferred embodiments and optional features, modification and variation of the concepts herein disclosed may be resorted to by those skilled in the art, and that such modifications and variations are considered to be within the scope of the appended claims.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

### Example 1

### Identification and Isolation of New, Specific Athlete-Associated Gut Microbes

To identify athlete-associated gut microbes, athletes were recruited and requested to provide stool samples before and after strenuous physical activity (exercise, training, performance, competition, etc.). For the collection of stool, athletes were provided with a 15 mL falcon tube with a 1 mL pipette tip inserted inside. Participants were instructed to dip the pipette tips into soiled toilet tissue, then place them back into the tubes and label the tubes with the date and time of collection. Samples were kept at 4° C. for short-term storage until sample pickup, at which point they were immediately placed onto dry ice, then transferred to a −80° C. freezer for long-term storage. Stool samples were thawed on ice and resuspended in 2-5 mL of PBS, 250 μL of which was used for DNA extraction using the Mo Bio PowerSoil high-throughput DNA extraction kit, following the manufacturer's protocol. For genus/OTU level identification, 16S ribosomal DNA (rDNA) analysis was conducted utilizing 1-5 μL of purified DNA for PCR amplification of the V4 variable region. 16S rDNA amplicon sequencing was followed by bioinformatic analysis, utilizing either open access software tools such as DADA2 and Qiime or custom scripts. For species/strain level identification, and gene functional annotation, whole genome shotgun analysis was conducted using 1 ng of purified DNA for Illumina's Nextera XT Tagmentation kit. Shotgun sequencing was followed by bioinformatic analysis, utilizing open access software tools such as Metaphlan, Humann, megahit, and prokka, or custom analysis scripts.

Through analysis of the sequencing data, the following strains were identified as being significantly more prevalent among the elite athletes, as compared to non-athlete controls, as well as enriched after strenuous physical activity: a Lactobacillus plantarum strain comprising a 16S rRNA gene comprising a nucleic acid sequence of SEQ ID NO: 1; a Lactobacillus rhamnosus strain comprising a 16S rRNA gene comprising a nucleic acid of SEQ ID NO: 21; a Lactobacillus acidophilus strain comprising a 16S rRNA gene comprising a nucleic acid of SEQ ID NO: 41; a Lactobacillus paracasei strain comprising a 16S rRNA gene comprising a nucleic acid of SEQ ID NO: 61; a Bifidobacterium longum strain comprising a 16S rRNA gene comprising a nucleic acid of SEQ ID NO: 81, SEQ ID NO: 201, or SEQ ID NO: 221; a Bifidobacterium animalis lactis strain comprising a 16S rRNA gene comprising a nucleic acid of SEQ ID NO: 101; a Veillonella dispar strain comprising a 16S rRNA gene comprising a nucleic acid sequence of SEQ ID NO: 121; a Veillonella parvula strain comprising a 16S rRNA gene comprising a nucleic acid of SEQ ID NO: 141; and a Veillonella atypica strain comprising a 16S rRNA gene comprising a nucleic acid of SEQ ID NO: 161. Lactobacillus species (L. plantarum, L. rhamnosus, L. acidophilus and L. paracasei), Bifidobacterium species (B. longum and B. animalis lactis), and Veillonella sp. (V. dispar, V. parvula, and V. atypica) were isolated and purified from several athletes by plating stool, resuspended in PBS, onto lactate agar petri dishes (per liter: 5 g bacto yeast extract, 0.75 g sodium thioglycolate, 25 mL basic fuchsin and 21 ml 60% sodium lactate, 15 g bacto agar, 7.5 ug/mL vancomycin (pH 7.5)) and grown under anaerobic conditions. Individuals colonies were selected and identified initially using MALDI-TOF mass spectrometry, followed by 16S rRNA and whole genome shotgun sequencing methodologies as described above. Lactobacillus, Bifidobacterium, and Veillonella species were then propagated and maintained in three different media compositions: (1) BHI supplemented with lactate(10 ml of 60% sodium lactate per liter); (2) MRS broth supplemented with lactate (10 ml of 60% sodium lactate per liter); and (3) lactate medium). Lactobacillus, Bifidobacterium, and Veillonella species were inoculated into each medium and grown under anaerobic conditions. These strains were deposited with the American Type Culture Collection (ATCC).

### Example 2

### Characterization of Athlete-Associated Veillonella atypica PTA-126860

The ability of Veillonella atypica PTA-126860 to convert lactate to propionate and acetate was observed by Mass Spectrometry. Veillonella atypica PTA-126860 was cultured in MRS lactate medium, which contains sodium lactate, a nutrient source for Veillonella strains, for 48 hours at 37° C. anaerobically. MRS lactate medium alone was used as a negative or baseline control. FIG. 4 shows that Veillonella atypica PTA-126860 produces both acetate and propionate from lactate, as compared to the baseline control. This result verifies that the newly identified Veillonella atypica PTA-126860 strain is capable of producing performance enhancing molecules, such as acetate and propionate. Therefore, this supports that the administration of Veillonella atypica PTA-126860 to a subject might improve athletic performance in the subject by increasing the levels of performance-enhancing molecules, such as acetate and propionate, in the subject.

### Example 3

### Characterization of Compositions Comprising Veillonella sp. and Lactate Producing Bacteria

Compositions comprising Veillonella atypica PTA-126860 and any one of the following lactate-producing microbes were generated: FB00015 Lactobacillus plantarum, FB00012 Lactobacillus acidophilus, FB00047 Lactobacillus rhamnosus, FB00032 Bifidobacterium longum, and FB00034 Bifidobacterium lactis. In addition, compositions comprising Veillonella atypica PTA-126860 and Mix 1 (comprising FB00015 L. plantarum, FB00012 L. acidophilus, FB00047 L. rhamnosus) or Mix 2 (comprising FB00015 L. plantarum, FB00012 L. acidophilus, FB00047 L. rhamnosus, FB00032 B. longum and FB00034 B. lactis) were also generated. Table 2 lists the 16S rRNA sequences of the additional strains in these compositions. For each of the compositions, equal amounts of each of the constituent strains were used. In other words, a ratio of 1:1 was used for a composition with 2 microbes, a ratio of 1:1:1:1 was used for a composition with 4 microbes and so on. The compositions were cultured in MRS lactate medium for 48 hours at 37° C. anaerobically. MRS lactate medium alone was used as a negative or baseline control. The ability of the compositions to convert lactate to propionate and acetate was observed by Mass Spectrometry.

TABLE 2

| | Sequence of |
|---|---|
| Strain | 16S rRNA |
| FB00015 L. plantarum | SEQ ID NO: 1 |
| FB00047 L. rhamnosus | SEQ ID NO: 21 |
| FB00012 L. acidophilus | SEQ ID NO: 41 |
| FB00032 B. longum | SEQ ID NO: 81 |
| FB00034 B. animalis lactis | SEQ ID NO: 101 |

Surprisingly, there is a striking increase in the amount of acetate produced by each of the tested compositions, as compared to Veillonella atypica PTA-126860 alone. For instance, the composition comprising Veillonella atypica PTA-126860 and Bifidobacterium longum produces >3 fold more acetate than Veillonella atypica PTA-126860 alone. Also, each of the compositions tested produced less propionate than Veillonella atypica PTA-126860 alone (FIG. 4). Overall, these results demonstrate that compositions comprising an Veillonella sp., such as, Veillonella atypica PTA-126860, and a lactate producing bacteria produce more acetate, as compared to the Veillonella atypica PTA-126860 strain on its own.

### Example 4

### Human Performance Testing of Athlete-Associated Lactobacillus sp.

The overall objective of the study was to assess consumer response and impact from consuming athlete-derived probiotics, as well as determine optimal dosing and formulations.

Subjects were required to live in the United States and ideally lead a healthy and active lifestyle. The subjects could not take prescription medications or have a medical condition that would impact or limit the ability to take the probiotic supplement. The subjects also could not take prescription medications or have a medical condition that would result in a compromised immune system. The subjects could not be pregnant.

Subjects received either a low or high dose of an oral composition comprising L. plantarum (FB00015; SEQ ID NO: 1), L. rhamnosus (FB00047; SEQ ID NO: 21), and L. acidophilus (FB00012; SEQ ID NO: 41) once per day over a 14 day period. The low dose comprised: 4×10⁹ CFU L. plantarum, 4×10⁹ CFU L. rhamnosus, and 2×10⁹ CFU L. acidophilus for a 10×10⁹ CFU total dose. The high dose comprised: 15×10⁹ CFU L. plantarum, 15×10⁹ CFU L. rhamnosus, and 5×10⁹ CFU L. acidophilus for a 35×10⁹ CFU total dose.

Subjects completed an intake form answering background questions on overall health and physical activity. Subjects were instructed to orally administer the probiotics daily for 14 days and maintain a normal exercise regimen and diet during the 14 day period. During the 14 day period, the subjects completed three surveys: (1) baseline, day 0; (2) week 1 assessment; and (3) week 2 assessment. The assessments were designed to determine how the subjects responded to probiotic supplementation. They also were used to help determine how often the subjects would use the probiotic supplement and in what formats and benefits they preferred. The assessments also gauged whether the subjects would be interested in such a product and would recommend the product to others.

Initial Beta Test Results (n=˜70 Subjects)

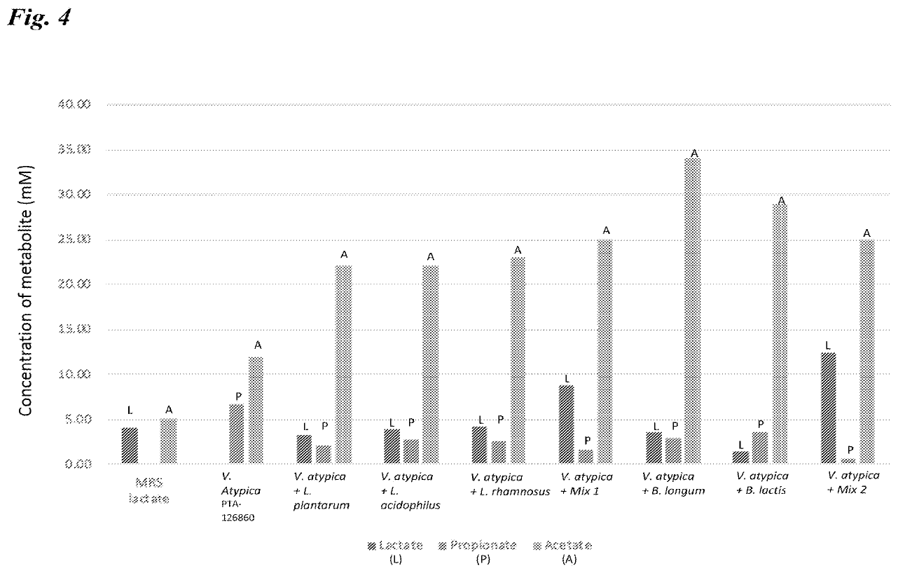

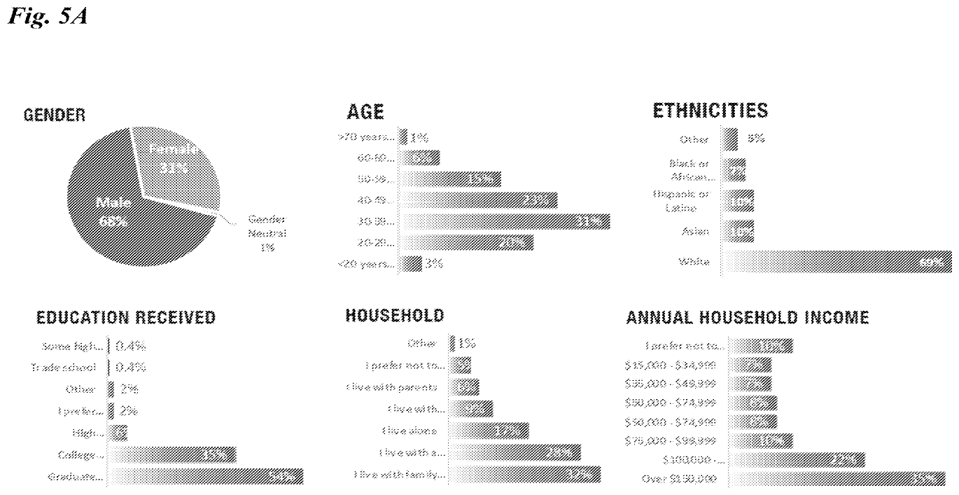

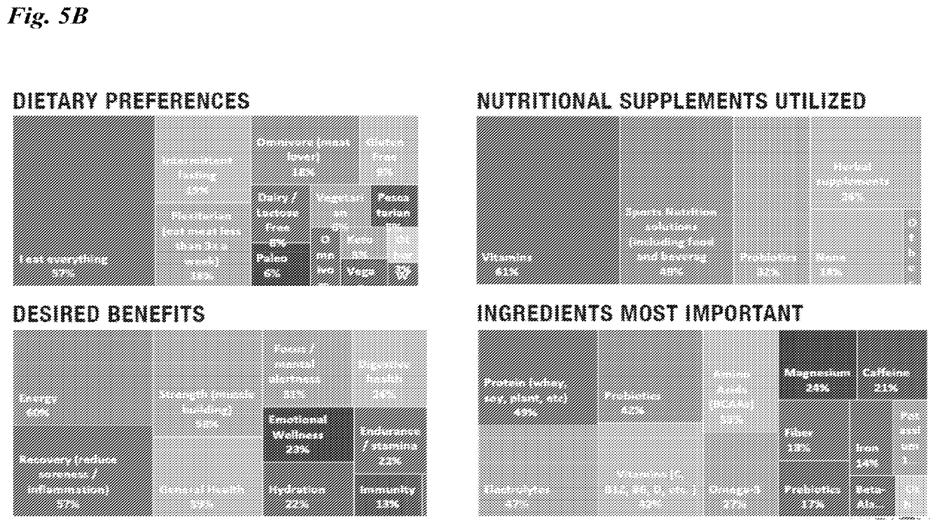

FIG. 5A provides a summary of the subject demographics. FIG. 5B shows the dietary preferences, nutritional supplements, and desired benefits and ingredients most important to the subjects.

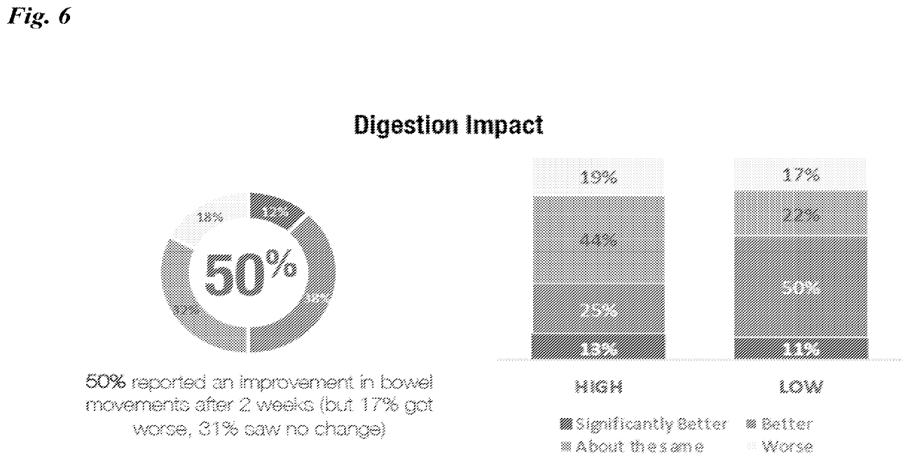

FIG. 6 shows the impact of low and high dose probiotic supplementation on digestion. Fifty percent of the subjects reported better or significantly better bowel movements after two weeks of supplementation compared to subjects at baseline.

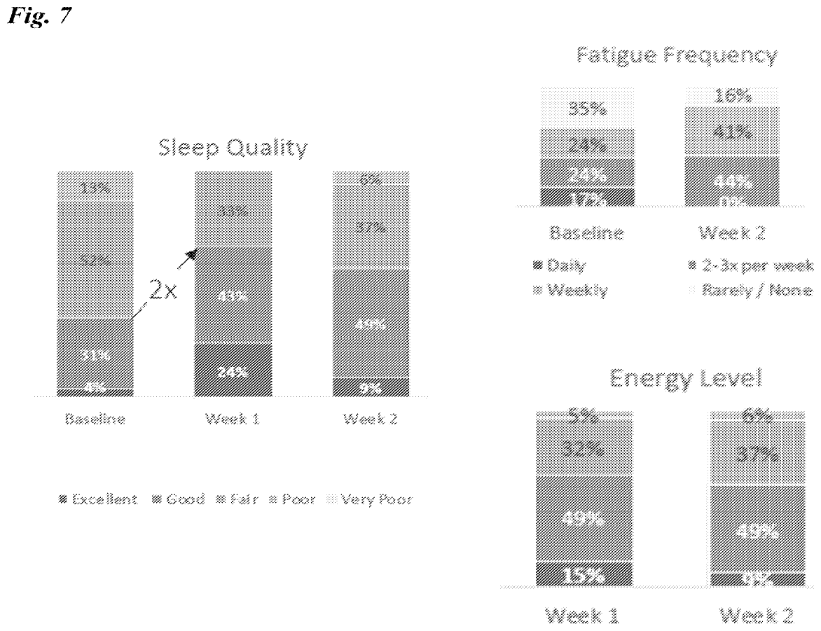

FIG. 7 shows the impact of probiotic supplementation on sleep quality, fatigue, and energy. Sleep quality improved nearly two-fold with 67% of subjects reporting excellent or good sleep after one week of supplementation compared to 35% of subjects at baseline. Daily fatigue decreased from 17% of subjects at baseline to 0% of subjects following two weeks of supplementation. Over 55% percent of subjects also reported excellent or good levels of energy after one or two weeks of supplementation. About half the respondents reported that these weeks were more stressful than normal (data not shown).

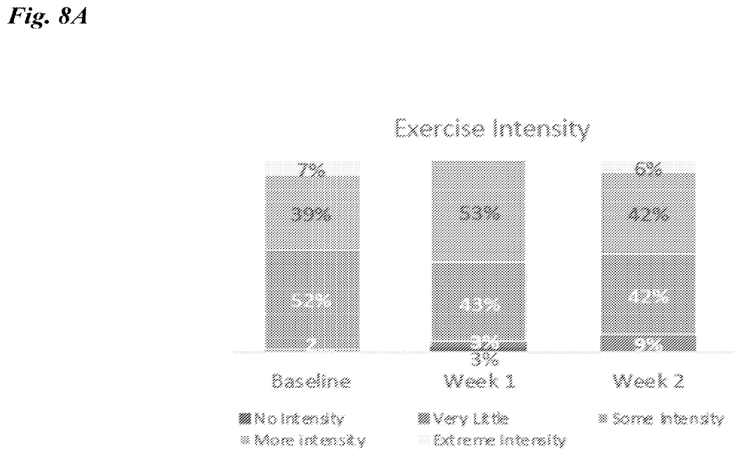

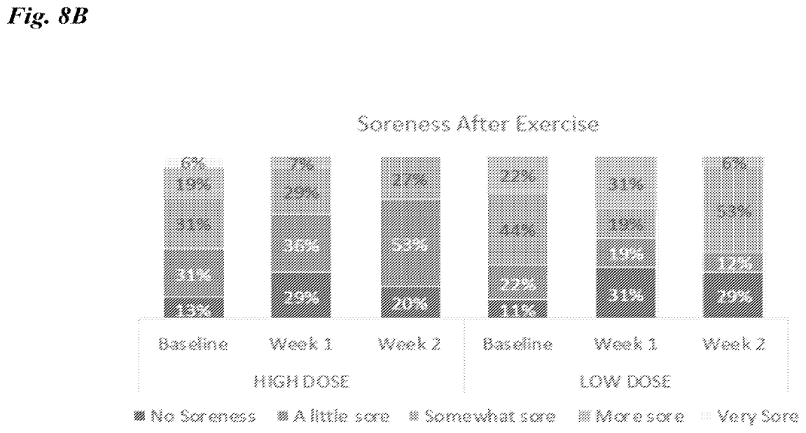

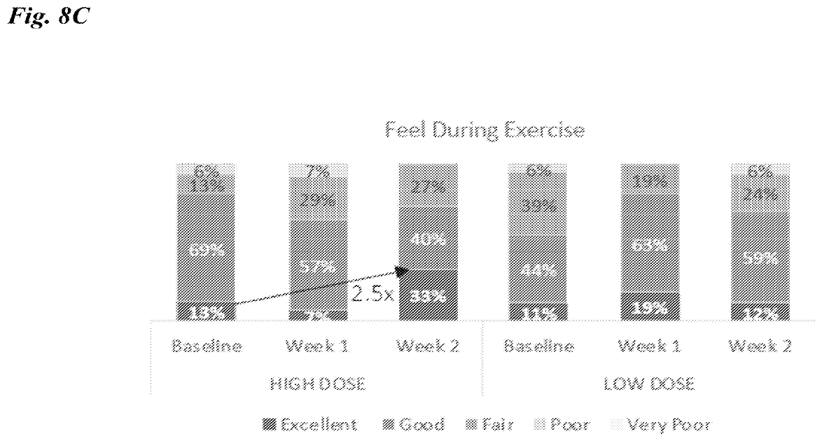

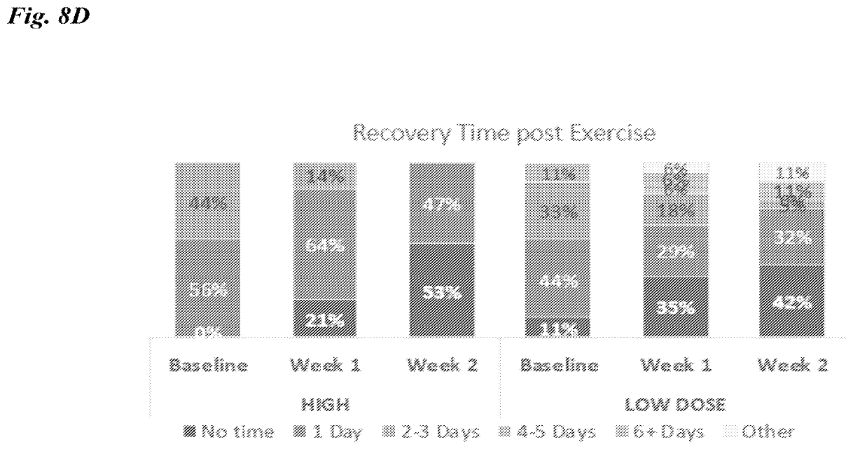

FIG. 8A shows exercise intensity of the subjects at baseline, one week, and two weeks following probiotic supplementation. Exercise intensity remained fairly consistent throughout the study with the majority of subjects reporting some or more exercise intensity.

FIG. 8B shows soreness after exercise of subjects at baseline, one week, and two weeks following low or high dose probiotic supplementation. Overall, over half of the subjects reported little to no soreness after two weeks of probiotic supplementation compared to subjects at baseline.

FIG. 8C shows the feeling during exercise of subjects at baseline, one week, and two weeks following low or high dose probiotic supplementation. Thirty-three percent of subjects reported feeling excellent with two weeks of high dose probiotic supplementation compared to only 13% of subjects at baseline.

FIG. 8D shows the recovery time after exercise of subjects at baseline, one week, and two weeks following low or high dose probiotic supplementation. There was a dramatic improvement in recovery after exercise. For example, 53% of subjects reported needing no time to recover from exercise following two weeks of high dose probiotic supplementation compared to 0% of subjects at baseline.

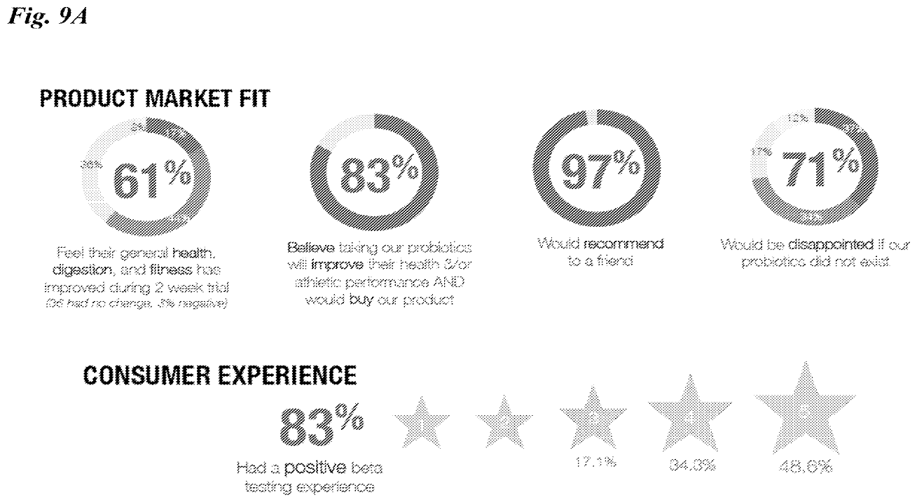

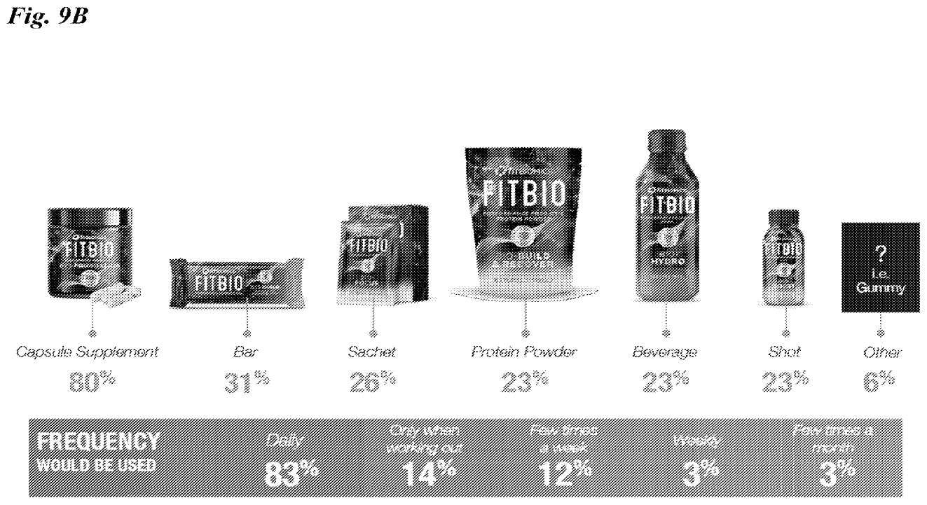

FIG. 9A provides an overall summary of the results from the human performance testing of probiotic supplementation. FIG. 9B shows the preferred format and frequency of use of the probiotic supplement for subjects.

Updated Beta Test Results (n=257 Subjects)

Two hundred and fifty seven subjects participated in the study with 129 subjects receiving a high dose of probiotics and 124 subjects receiving a low dose of probiotics daily for 14 days. Doses of microorganisms described above.

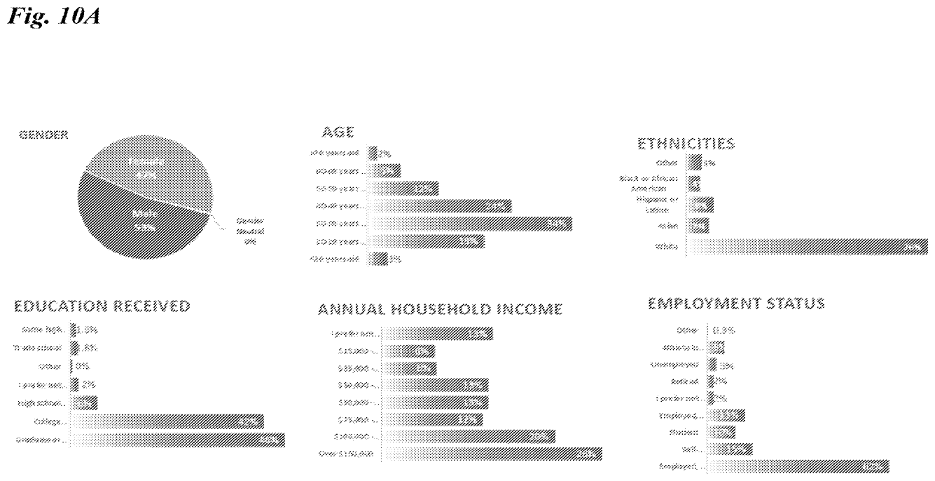

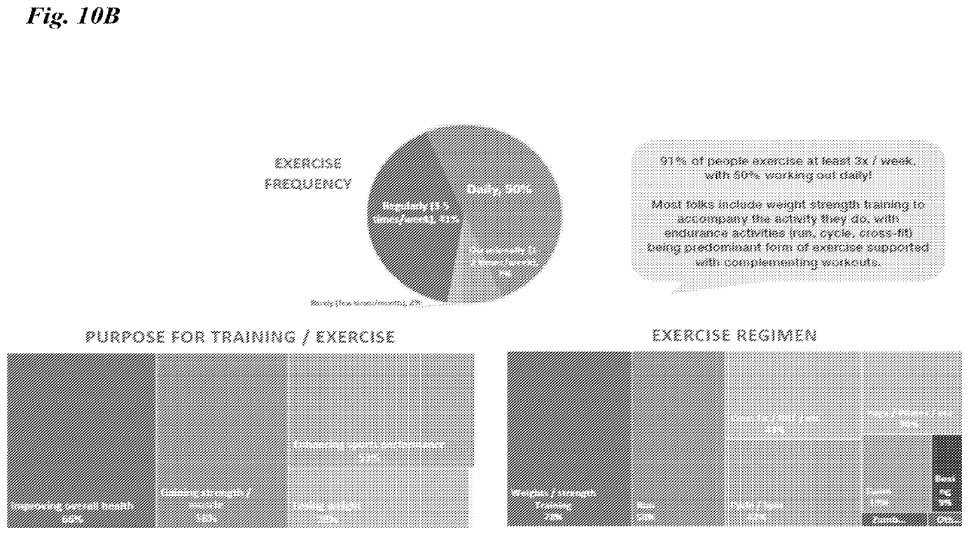

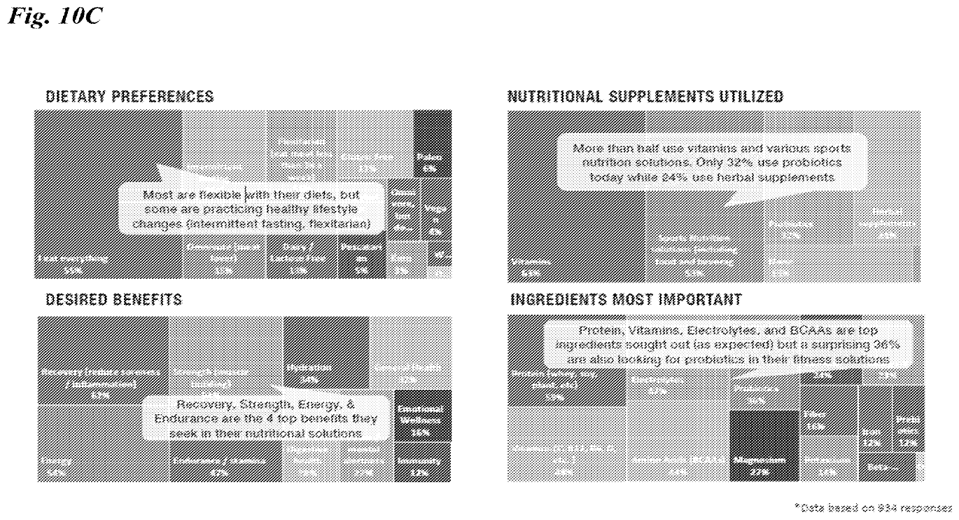

FIG. 10A provides a summary of the subject demographics. FIG. 10B shows exercise frequency, exercise regimen, and purpose of training and exercise of the subjects. FIG. 10C shows the dietary preferences, nutritional supplements, and desired benefits and ingredients most important to the subjects.

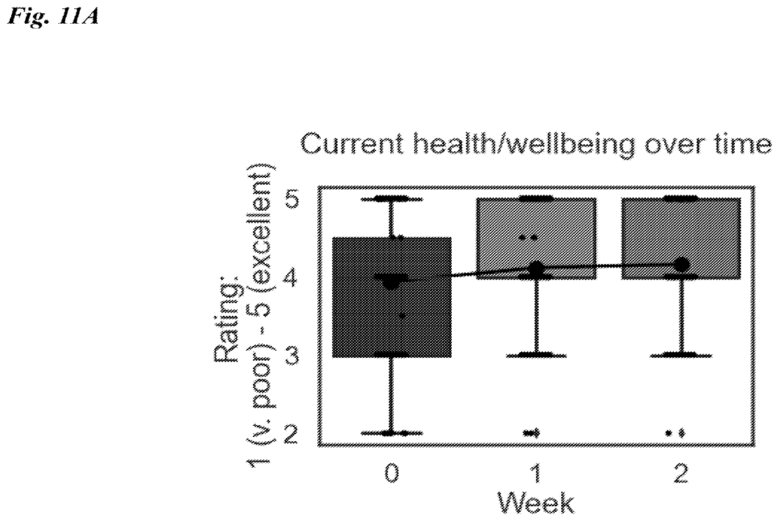

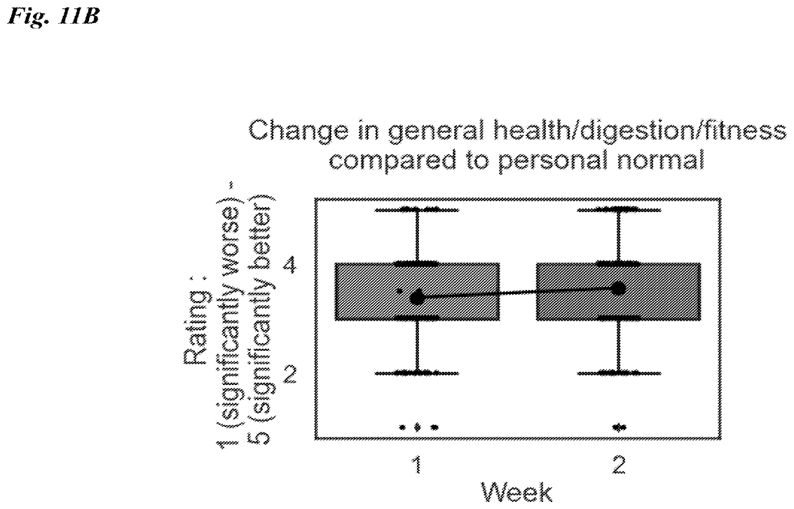

FIG. 11A shows the impact of probiotic supplementation on current health and wellbeing overtime. FIG. 11B shows the impact of probiotic supplementation on general health, digestion, and fitness compared to personal normal.

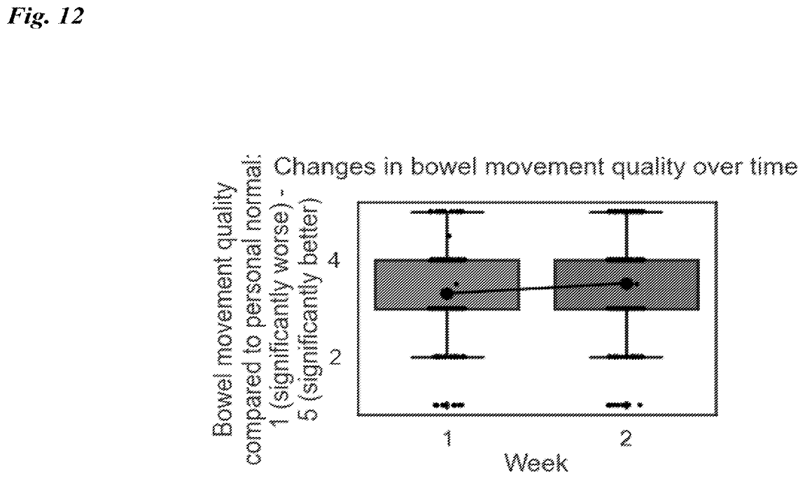

FIG. 12 shows the impact of probiotic supplementation on the change in bowel movement quality over time.

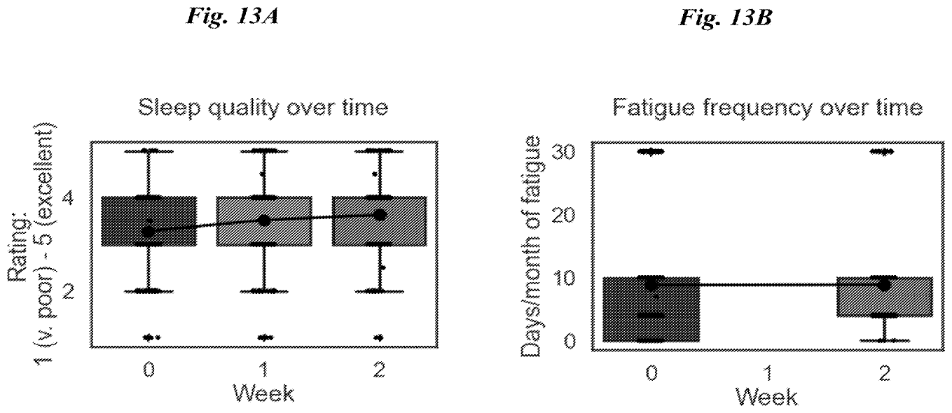

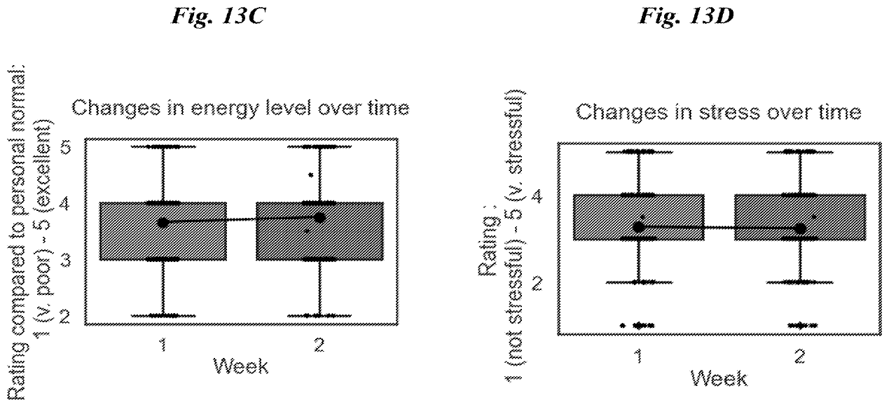

FIG. 13A shows the impact of probiotic supplementation on sleep quality overtime. FIG. 13B shows the impact of probiotic supplementation on fatigue frequency overtime. FIG. 13C shows the impact of probiotic supplementation on energy level overtime. FIG. 13D shows the impact of probiotic supplementation on stress level overtime.

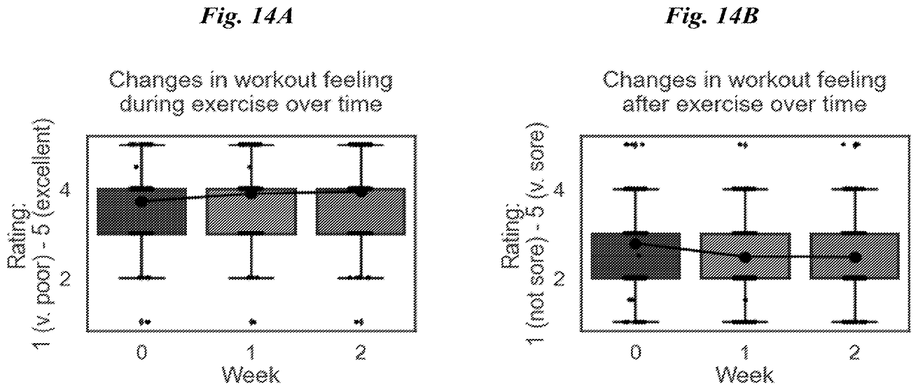

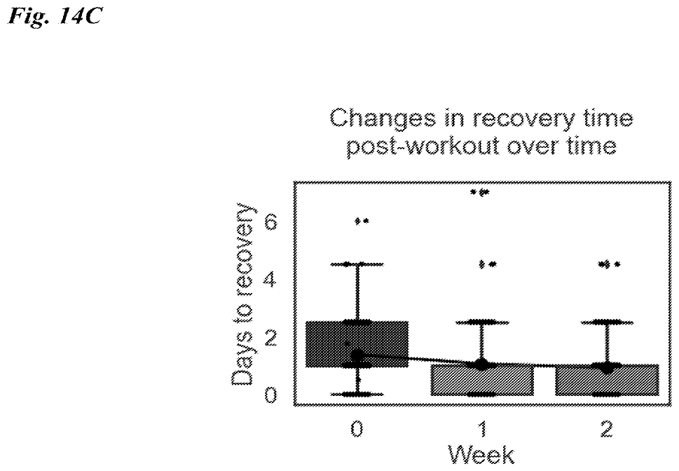

FIG. 14A shows the feeling during exercise of subjects at baseline, one week, and two weeks following probiotic supplementation. FIG. 14B shows the feeling after exercise of subjects at baseline, one week, and two weeks following probiotic supplementation. FIG. 14C shows the recovery time after exercise of subjects at baseline, one week, and two weeks following probiotic supplementation.

A summary of the effect of probiotic supplementation on the different functional categories described above are provided in Table 3 below.

TABLE 3

| | | | | Fold | |
|---|---|---|---|---|---|
| | | | Difference | change | |
| | Week 0 - | Week 0 - | mean | mean | % change |
| | Week 1 | Week 2 | Week 2 - | Week 2 - | Week 2 - |
| Feature | p-value | p-value | Week 0 | Week 0 | Week 0 |
| Change in | 0.011 | 0.002 | 0.220 | 1.056 | 5.6% |
| health/ | | | | | |
| wellbeing | | | | | |
| Sleep | 0.001 | <0.001 | 0.347 | 1.106 | 10.7% |
| quality | | | | | |
| Fatigue | NA | <0.001 | 0.041 | 1.004 | 0.5% |
| frequency | | | | | |
| Workout | 0.006 | <0.001 | 0.223 | 1.060 | 5.9% |
| feeling | | | | | |
| during | | | | | |
| Workout | <0.001 | <0.001 | −0.305 | 0.890 | 10.8% |
| feeling | | | | | |
| after | | | | | |
| Recovery | <0.001 | <0.001 | −0.349 | 0.727 | 33.6% |
| time (dys) | | | | | |
| Change in | Week | NA | 0.212 | 1.064 | Week |
| bowel | 1-2: 0.001 | | | | 1-2: 6.3% |
| movements | | | | | |
| Change in | Week | NA | 0.093 | 1.025 | Week |
| energy | 1-2: >0.05 | | | | 1-2: 2.5% |
| level | | | | | |
| Change in | Week | NA | −0.035 | 0.989 | Week |
| stress | 1-2: >0.05 | | | | 1-2: 1.2% |
| Change in | Week | NA | 0.171 | 1.051 | Week |
| health/ | 1-2: 0.017 | | | | 1-2: 5.0% |
| digestion/ | | | | | |
| fitness | | | | | |

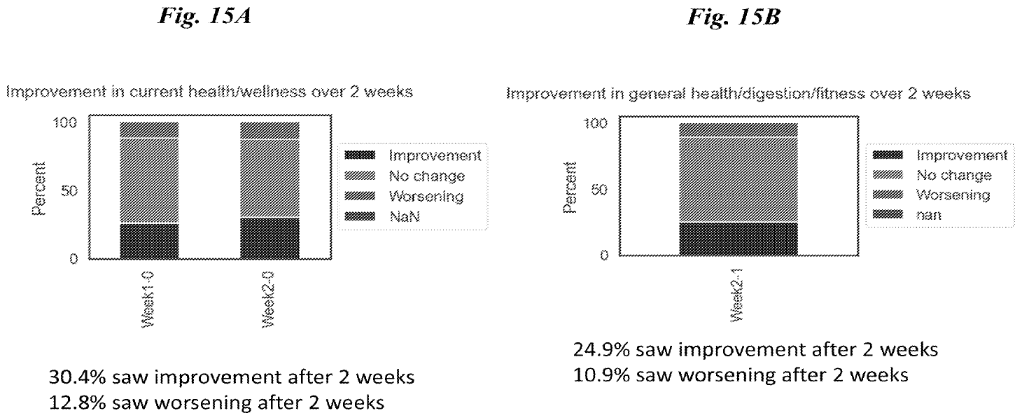

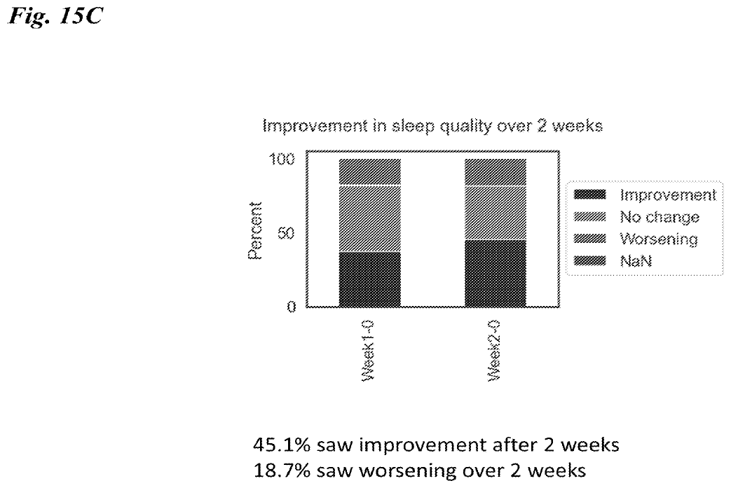

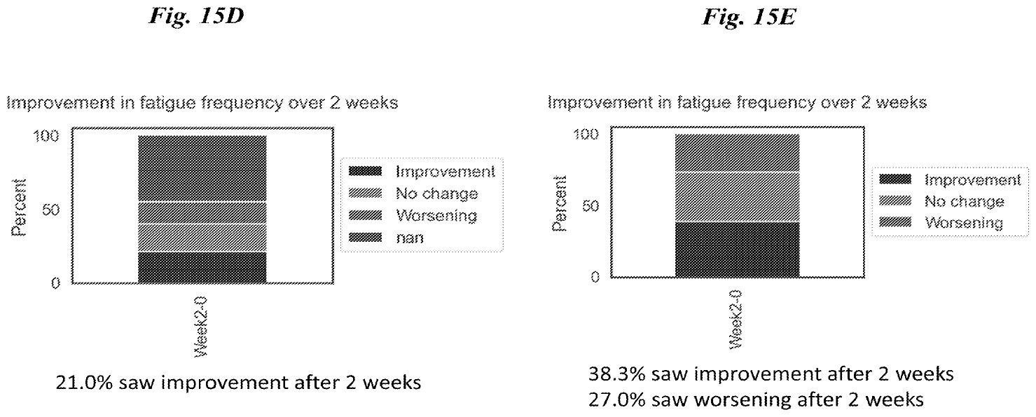

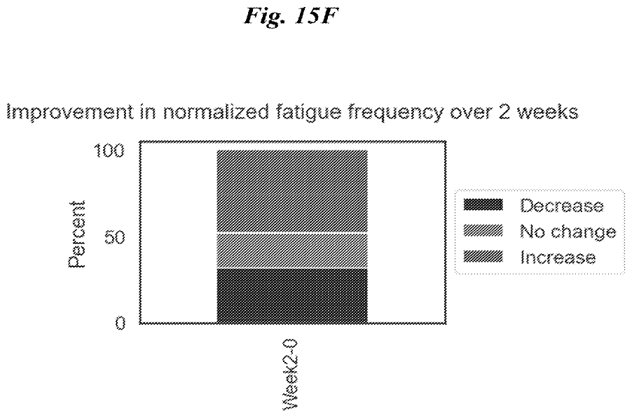

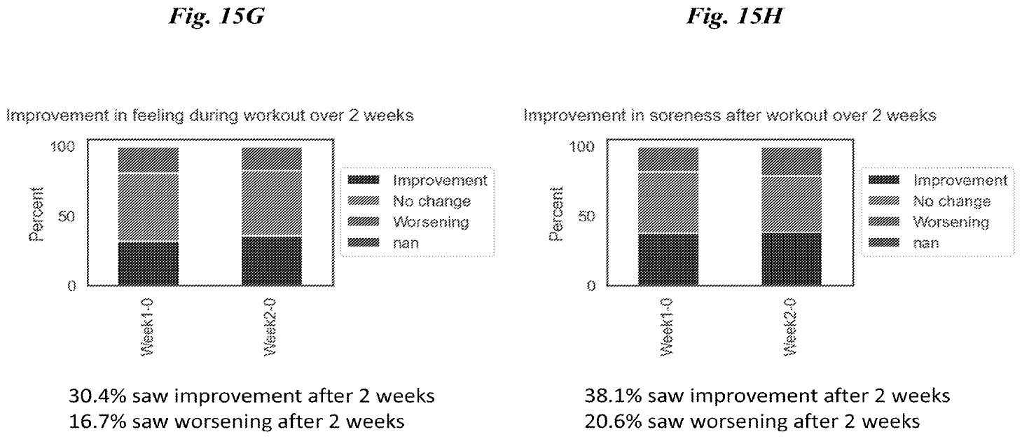

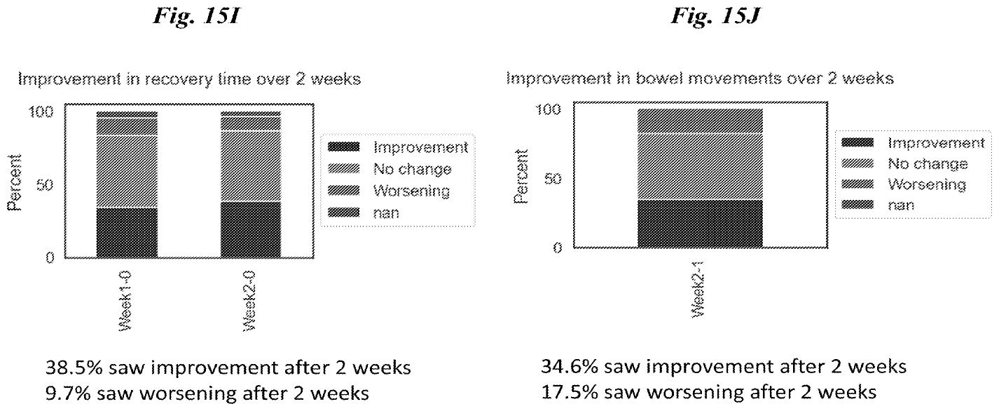

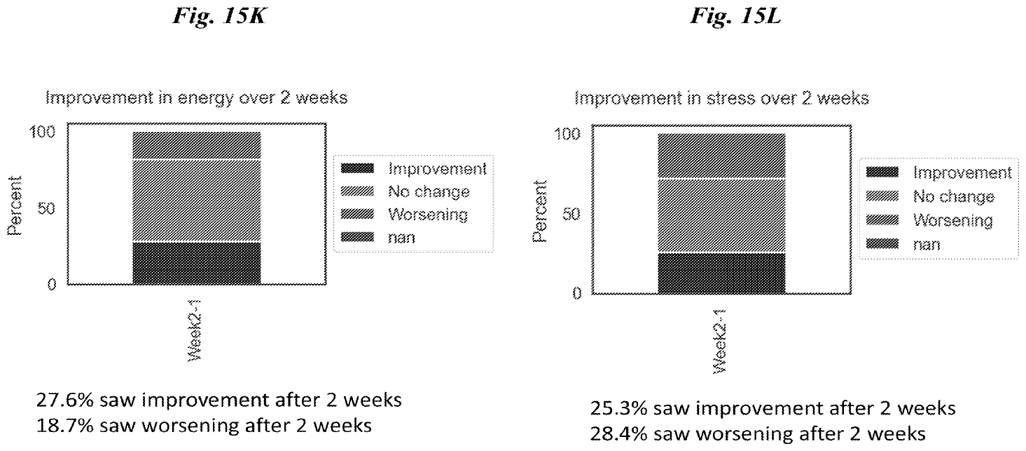

FIG. 15A shows the improvement in current health and wellness in subjects following one or two weeks of probiotic supplementation. FIG. 15B shows the improvement in general health, digestion, and fitness in subjects following two weeks of probiotic supplementation.

FIG. 15C shows the improvement in sleep quality in subjects following one or two weeks of probiotic supplementation. FIG. 15D shows the improvement in fatigue frequency in subjects following two weeks of probiotic supplementation. FIG. 15E shows the improvement in fatigue frequency in subjects following two weeks of probiotic supplementation. FIG. 15F and Table 4 shows the improvement in normalized fatigue frequency in subjects following two weeks of probiotic supplementation. Fatigue frequency was normalized to workout intensity.

TABLE 4

| | Percent change | Participants | |
|---|---|---|---|
| | in mean value | who saw fatigue | |
| | from week 0 | decrease over | |
| | to week 2 | two weeks (%) | P value |
| Fatigue Frequency | 0.5% increase | 26.95% | 1.39E−15 |
| Workout intensity | 2.4% decrease | NA | 0.3 (not |
| | | | significant) |
| Fatigue frequency | 5.3% increase | 31.65% | 4.64E−16 |
| normalized to | | | |
| workout intensity | | | |

FIG. 15G shows the improvement in feeling during a workout in subjects following one or two weeks of probiotic supplementation. FIG. 15H shows the improvement in soreness in subjects following one or two weeks of probiotic supplementation. FIG. 15I shows the improvement in recovery time after a workout in subjects following one or two weeks of probiotic supplementation. FIG. 15J shows the improvement in bowel movements in subjects following two weeks of probiotic supplementation. FIG. 15K shows the improvement in energy in subjects following two weeks of probiotic supplementation. FIG. 15L shows the improvement in stress in subjects following two weeks of probiotic supplementation.

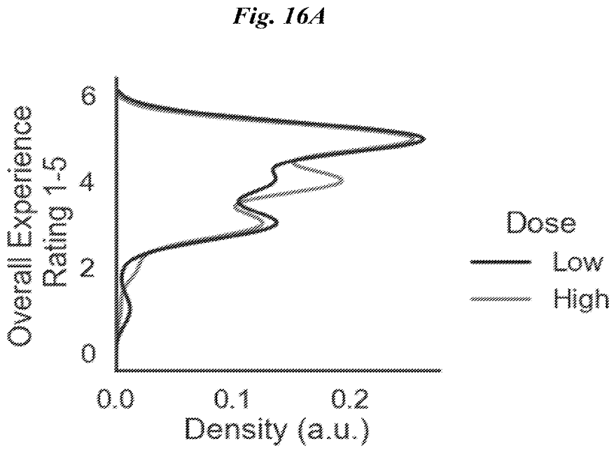

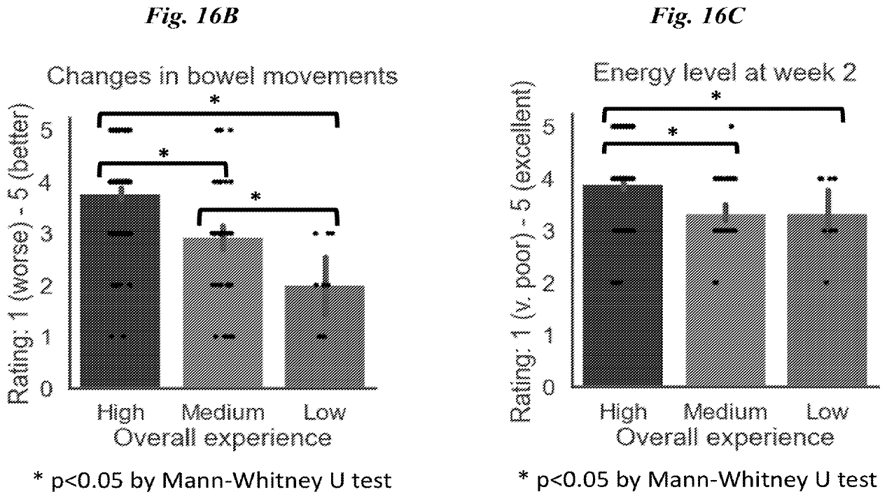

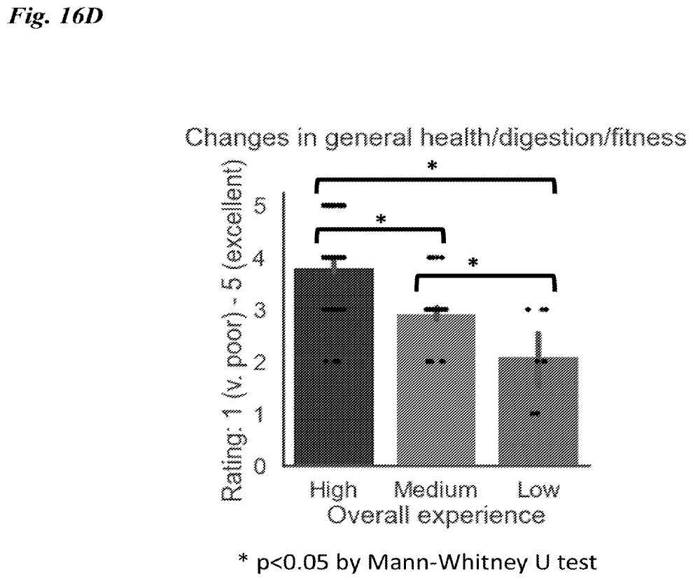

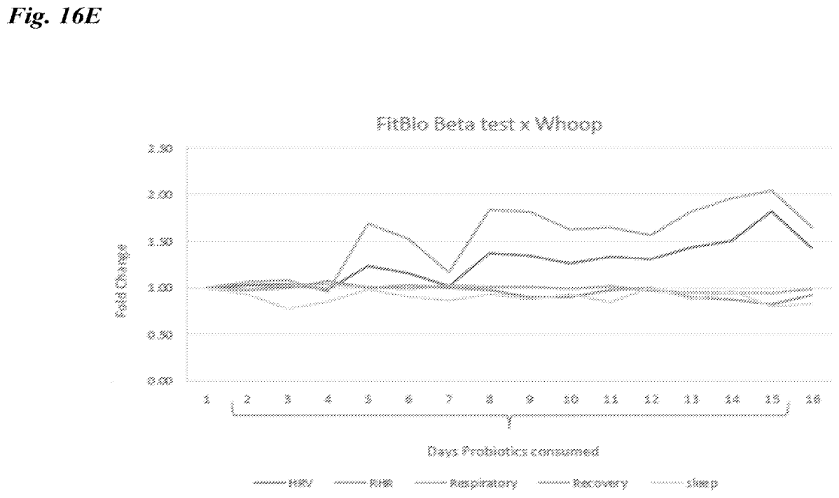

FIG. 16A shows the overall experience of subjects in the study that received low or high dose probiotic supplementation. Overall experience was measured on a scale of 1 to 5 with 1 as very poor and 5 as excellent. Most subjects reported their overall experience as a 4 or 5 with no significant difference between low and high dose probiotic supplementation. FIG. 16B shows the changes in bowel movements following probiotic supplementation in subjects with high, medium, or low overall experience. FIG. 16C shows the energy level at week 2 of probiotic supplementation in subjects with high, medium, or low overall experience. FIG. 16D shows the changes in general health, digestion, and fitness following probiotic supplementation in subjects with high, medium, or low overall experience. FIG. 16E shows heart rate variability (HRV), resting heart rate (RHR), respiratory, recovery, and sleep scores in subjects receiving probiotic supplementation overtime. Results were measured using the WHOOP fitness tracking devise.

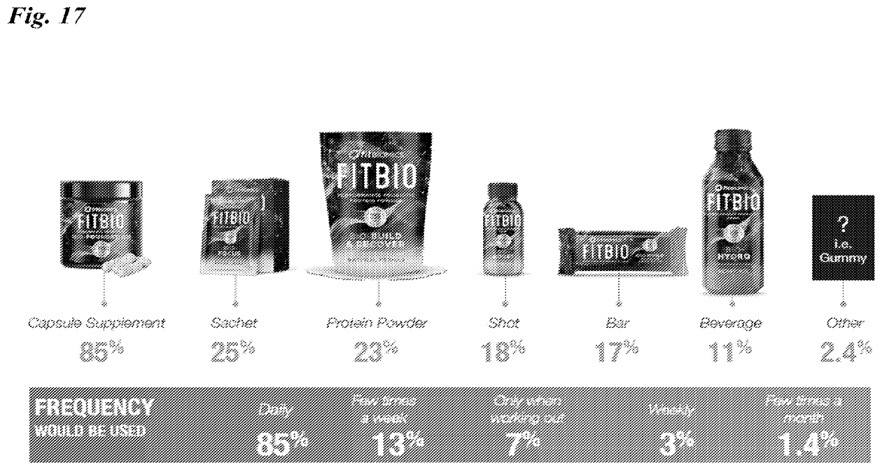

FIG. 17 shows the preferred format and frequency of use of the probiotic supplement for subjects.

### Example 5

### Characterization of Athlete-Associated Gut Microbes In Vivo

Human feasibility studies will be performed to determine Lactobacillus, Bilidobacterium, and/or Veillonella species GI transit time and viability, as well as lactate metabolism functionality via stool metagenomic and metabolomic analysis. This will be followed by human performance testing using double blind placebo controlled cross over studies to determine the effect of Lactobacillus, Bilidobacterium, and/or Veillonella species consumption on VO2 max, exercise capacity, endurance, inflammation, recovery, and strength applications. Lactobacillus, Bifidobacterium, and/or Veillonella species will be tested in different doses as well as in combination with potentially synergistic compounds (vitamins, electrolytes, prebiotics, lactate, prebiotics, etc.). Pre-clinical studies will be performed in mice to explore additional functional benefits of Lactobacillus, Bifidobacterium, and/or Veillonella species, including: 1) strength and protein metabolism/absorption; 2) neurological and gut-brain access applications (sleep, stress, anxiety social interactions, cognition); 3) inflammation (gut and distal location in the body); 4) immunity; and 5) nutrition and digestion.

### (1) Human Performance Testing of Veillonella Strains

A cohort of human subjects (for example, ˜50 subjects) will be administered about 5-10 billion CFU of either one, or any combination of, the following bacteria: Veillonella atypica having deposit accession number PTA-126860, Veillonella dispar having deposit accession number PTA-126861, and Veillonella parvula having deposit accession number PTA-126859.

Health indicators (such as, the diversity of the microbiome, the blood levels of lactates, acetates and propionates, exercise endurance, athletic performance, levels of inflammatory cytokines and other markers of inflammation, ability to recover from physical exercise, muscle mass or muscular strength, sleep quality, sleep length, and mental health indicators) will be measured for these subjects before and after administration of the Veillonella strains for a period of time (e.g., 2 weeks). A comparison of these health indicators will also be done with human subjects who were administered a placebo over this time period.

After administration of the aforementioned Veillonella strains, human subjects will show any one of: an altered microbiome, improved diversity of microbiome, reduced blood levels of lactates, increased blood levels of propionates and/or acetates, enhanced exercise endurance, improved athletic performance, reduced inflammation, reduced levels of inflammatory cytokines and/or other markers of inflammation, enhanced ability to recover from physical exercise, increased muscle mass or muscular strength, decreased muscle loss, better quality of sleep, longer sleep, and/or improved mental health indicators, and/or any combination thereof, as compared to before administration, or human subjects on placebo.

(2) Human Performance Testing of Veillonella Strains in Combination with Lactate Producing Bacteria

A cohort of human subjects (for example, ˜50 subjects) will be administered about 5-10 billion CFU of: (a) Veillonella atypica having deposit accession number PTA-126860, Veillonella dispar having deposit accession number PTA-126861, and/or Veillonella parvula having deposit accession number PTA-126859; and (b) Lactobacillus plantarum comprising a 16S rRNA nucleic acid sequence that is at least 97% identical to SEQ ID NO: 1; Lactobacillus acidophilus comprising a 16S rRNA nucleic acid sequence that is at least 97% identical to SEQ ID NO: 41; Lactobacillus rhamnosus comprising a 16S rRNA nucleic acid sequence that is at least 97% identical to SEQ ID NO: 21; Lactobacillus paracasei comprising a 16S rRNA nucleic acid sequence that is at least 97% identical to SEQ ID NO: 61 or SEQ ID NO: 181; Bifidobacterium longum comprising a 16S rRNA nucleic acid sequence that is at least 97% identical to SEQ ID NO: 81, SEQ ID NO: 201, or SEQ ID NO: 221; and/or Bifidobacterium lactis comprising a 16S rRNA nucleic acid sequence that is at least 97% identical to SEQ ID NO: 101.

For example, one cohort of human subjects will be administered Veillonella atypica having deposit accession number PTA-126860, in combination with aforementioned Lactobacillus and Bifidobacterium strains. As another example, another cohort of human subjects will be administered Veillonella atypica having deposit accession number PTA-126860, in combination with just the aforementioned Lactobacillus strains.

Health indicators (such as, the diversity of the microbiome, the blood levels of lactates, acetates and propionates, exercise endurance, athletic performance, levels of inflammatory cytokines and other markers of inflammation, ability to recover from physical exercise, muscle mass or muscular strength, sleep quality, sleep length, and mental health indicators) will be measured for these subjects before and after administration of the microbes or compositions described herein for a period of time (e.g., 2 weeks). A comparison of these health indicators will also be done with human subjects who were administered a placebo, and with human subjects who were administered just Veillonella strains over this time period.

After administration of the aforementioned combinations, human subjects will show any one of: an altered microbiome, improved diversity of microbiome, reduced blood levels of lactates, increased blood levels of propionates and/or acetates, enhanced exercise endurance, improved athletic performance, reduced inflammation, reduced levels of inflammatory cytokines and/or other markers of inflammation, enhanced ability to recover from physical exercise, increased muscle mass or muscular strength, decreased muscle loss, better quality of sleep, longer sleep, and/or improved mental health indicators, and/or any combination thereof, as compared to before administration, or human subjects on placebo, or human subjects who were administered just Veillonella strains.

### Example 6

### Characterization of Metabolic Pathways in Athlete-Associated Gut Microbes

Dozens of additional athlete-associated Lactobacillus, Bifodobacterium, and Veillonella species and strains (variations of L. plantarum, L. rhamnosus, L. acidophilus, L. paracasei, B. longum, B. animalis lactis, V. dispar, V. parvula, and V. atypica) will be identified and isolated for comprehensive metagenomic functional annotation/analysis to identify genes and metabolic pathways contributing to beneficial growth, SCFA synthesis, energy and protein metabolism, immunological and digestive health, as well as other beneficial physiological processes. Annotated genes will be used to develop probiotic, nutritional, fitness, health and wellness, as well as synthetic biology markers and tools. Lactobacillus, Bifidobacterium, and Veillonella species will be grown in various culture media (BHI, MRS, lactate, etc.) to assess beneficial functions in terms of metabolite, neurotransmitter, lipid, and proteomic production as well as metabolic, enzymatic, fermentation, and growth capabilities. Lactobacillus, Bilidobacterium, and Veillonella species will be grown in conjunction with additional athlete associated gut microbes, substrates, nutrients, and prebiotics to look for synergistic affects. Mass spectrometry (MS) will be performed on the cultured media to determine the identity of the compounds that are produced by the athlete-associated gut microbes. It is expected that the athlete-associated gut microbes identified here will metabolize lactates in the media to produce products, such as acetate and propionates, which can be identified by MS.

### Further Numbered Embodiments

Embodiment 1. An isolated and purified Lactobacillus plantarum, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 2; (ii) VR2 comprises a nucleic acid sequence with at least 95% sequence identity to SEQ ID NO: 3; (iii) VR3 comprises a nucleic acid sequence with at least 95% sequence identity to SEQ ID NO: 4; (iv) VR4 comprises a nucleic acid sequence with at least 95% sequence identity to SEQ ID NO: 5; (v) VR5 comprises a nucleic acid sequence with at least 95% sequence identity to SEQ ID NO: 6; (vi) VR6 comprises a nucleic acid sequence with at least 95% sequence identity to SEQ ID NO: 7; (vii) VR7 comprises a nucleic acid sequence with at least 95% sequence identity to SEQ ID NO: 8; (viii) VR8 comprises a nucleic acid sequence with at least 95% sequence identity to SEQ ID NO: 9; and (ix) VR9 comprises a nucleic acid sequence with at least 95% sequence identity to SEQ ID NO: 10.

Embodiment 2. The Lactobacillus plantarum of embodiment 1, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 11; (ii) CR2 comprises a nucleic acid sequence with at least 80% sequence identity to SEQ ID NO: 12; (iii) CR3 comprises a nucleic acid sequence with at least 80% sequence identity to SEQ ID NO: 13; (iv) CR4 comprises a nucleic acid sequence with at least 80% sequence identity to SEQ ID NO:14; (v) CR5 comprises a nucleic acid sequence with at least 80% sequence identity to SEQ ID NO: 15; (vi) CR6 comprises a nucleic acid sequence with at least 80% sequence identity to SEQ ID NO: 16; (vii) CR7 comprises a nucleic acid sequence with at least 80% sequence identity to SEQ ID NO: 17; (viii) CR8 comprises a nucleic acid sequence with at least 80% sequence identity to SEQ ID NO: 18; (ix) CR9 comprises a nucleic acid sequence with at least 80% sequence identity to SEQ ID NO: 19; and (x) CR10 comprises a nucleic acid sequence with at least 80% sequence identity to SEQ ID NO: 20.

Embodiment 3. An isolated and purified Lactobacillus plantarum, comprising: a 16S rRNA gene comprising a nucleic acid sequence with at least 97% sequence identity to SEQ ID NO: 1.

Embodiment 4. An isolated and purified Lactobacillus plantarum having deposit accession number 202103009, or a strain having all of the identifying characteristics of Lactobacillus plantarum 202103009, or a mutant thereof.

Embodiment 5. An isolated and purified Lactobacillus rhamnosus, comprising: a 16S rRNA gene comprising a nucleic acid sequence comprising at least one variable region (VR) selected from the group consisting of VR1, VR2, VR3, VR4, VR5, VR6, VR7, VR8, VR9, and any combination thereof, wherein (i) VR1 comprises a nucleic acid sequence with at least 95% sequence identity to SEQ ID NO: 22; (ii) VR2 comprises a nucleic acid sequence with at least 95% sequence identity to SEQ ID NO: 23; (iii) VR3 comprises a nucleic acid sequence with at least 95% sequence identity to SEQ ID NO: 24; (iv) VR4 comprises a nucleic acid sequence with at least 95% sequence identity to SEQ ID NO: 25; (v) VR5 comprises a nucleic acid sequence with at least 95% sequence identity to SEQ ID NO: 26; (vi) VR6 comprises a nucleic acid sequence with at least 95% sequence identity to SEQ ID NO: 27; (vii) VR7 comprises a nucleic acid sequence with at least 95% sequence identity to SEQ ID NO: 28; (viii) VR8 comprises a nucleic acid sequence with at least 95% sequence identity to SEQ ID NO: 29; and (ix) VR9 comprises a nucleic acid sequence with at least 95% sequence identity to SEQ ID NO: 30.

Embodiment 6. The Lactobacillus rhamnosus of embodiment 5, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 31; (ii) CR2 comprises a nucleic acid sequence with at least 80% sequence identity to SEQ ID NO: 32; (iii) CR3 comprises a nucleic acid sequence with at least 80% sequence identity to SEQ ID NO: 33; (iv) CR4 comprises a nucleic acid sequence with at least 80% sequence identity to SEQ ID NO: 34; (v) CR5 comprises a nucleic acid sequence with at least 80% sequence identity to SEQ ID NO: 35; (vi) CR6 comprises a nucleic acid sequence with at least 80% sequence identity to SEQ ID NO: 36; (vii) CR7 comprises a nucleic acid sequence with at least 80% sequence identity to SEQ ID NO: 37; (viii) CR8 comprises a nucleic acid sequence with at least 80% sequence identity to SEQ ID NO: 38; (ix) CR9 comprises a nucleic acid sequence with at least 80% sequence identity to SEQ ID NO: 39; and (x) CR10 comprises a nucleic acid sequence with at least 80% sequence identity to SEQ ID NO: 40.

Embodiment 7. An isolated and purified Lactobacillus rhamnosus, comprising: a 16S rRNA gene comprising a nucleic acid sequence with at least 97% sequence identity to SEQ ID NO: 21.

Embodiment 8. An isolated and purified Lactobacillus rhamnosus having deposit accession number 202103010, or a strain having all of the identifying characteristics of Lactobacillus rhamnosus 202103010, or a mutant thereof.

Embodiment 9. An isolated and purified Lactobacillus acidophilus, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 42; (ii) VR2 comprises a nucleic acid sequence with at least 95% sequence identity to SEQ ID NO: 43; (iii) VR3 comprises a nucleic acid sequence with at least 95% sequence identity to SEQ ID NO: 44; (iv) VR4 comprises a nucleic acid sequence with at least 95% sequence identity to SEQ ID NO: 45; (v) VR5 comprises a nucleic acid sequence with at least 95% sequence identity to SEQ ID NO: 46; (vi) VR6 comprises a nucleic acid sequence with at least 95% sequence identity to SEQ ID NO: 47; (vii) VR7 comprises a nucleic acid sequence with at least 95% sequence identity to SEQ ID NO: 48; (viii) VR8 comprises a nucleic acid sequence with at least 95% sequence identity to SEQ ID NO: 49; and (ix) VR9 comprises a nucleic acid sequence with at least 95% sequence identity to SEQ ID NO: 50.

Embodiment 10. The Lactobacillus acidophilus of embodiment 9, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 51; (ii) CR2 comprises a nucleic acid sequence with at least 80% sequence identity to SEQ ID NO: 52; (iii) CR3 comprises a nucleic acid sequence with at least 80% sequence identity to SEQ ID NO: 53; (iv) CR4 comprises a nucleic acid sequence with at least 80% sequence identity to SEQ ID NO: 54; (v) CR5 comprises a nucleic acid sequence with at least 80% sequence identity to SEQ ID NO: 55; (vi) CR6 comprises a nucleic acid sequence with at least 80% sequence identity to SEQ ID NO: 56; (vii) CR7 comprises a nucleic acid sequence with at least 80% sequence identity to SEQ ID NO: 57; (viii) CR8 comprises a nucleic acid sequence with at least 80% sequence identity to SEQ ID NO: 58; (ix) CR9 comprises a nucleic acid sequence with at least 80% sequence identity to SEQ ID NO: 59; and (x) CR10 comprises a nucleic acid sequence with at least 80% sequence identity to SEQ ID NO: 60.

Embodiment 11. An isolated and purified Lactobacillus acidophilus, comprising: a 16S rRNA gene comprising a nucleic acid sequence with at least 97% sequence identity to SEQ ID NO: 41.

Embodiment 12. An isolated and purified Lactobacillus acidophilus having deposit accession number 202103008, or a strain having all of the identifying characteristics of Lactobacillus acidophilus 202103008, or a mutant thereof.

Embodiment 13. An isolated and purified Lactobacillus paracasei, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 62 or SEQ ID NO: 182; (ii) VR2 comprises a nucleic acid sequence with at least 95% sequence identity to SEQ ID NO: 63 or SEQ ID NO: 183; (iii) VR3 comprises a nucleic acid sequence with at least 95% sequence identity to SEQ ID NO: 64 or SEQ ID NO: 184; (iv) VR4 comprises a nucleic acid sequence with at least 95% sequence identity to SEQ ID NO: 65 or SEQ ID NO: 185; (v) VR5 comprises a nucleic acid sequence with at least 95% sequence identity to SEQ ID NO: 66 or SEQ ID NO: 186; (vi) VR6 comprises a nucleic acid sequence with at least 95% sequence identity to SEQ ID NO: 67 or SEQ ID NO: 187; (vii) VR7 comprises a nucleic acid sequence with at least 95% sequence identity to SEQ ID NO: 68 or SEQ ID NO: 188; (viii) VR8 comprises a nucleic acid sequence with at least 95% sequence identity to SEQ ID NO: 69 or SEQ ID NO: 189; and (ix) VR9 comprises a nucleic acid sequence with at least 95% sequence identity to SEQ ID NO: 70 or SEQ ID NO: 190.

Embodiment 14. The Lactobacillus paracasei of embodiment 13, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 71 or SEQ ID NO: 191; (ii) CR2 comprises a nucleic acid sequence with at least 80% sequence identity to SEQ ID NO: 72 or SEQ ID NO: 192; (iii) CR3 comprises a nucleic acid sequence with at least 80% sequence identity to SEQ ID NO: 73 or SEQ ID NO: 193; (iv) CR4 comprises a nucleic acid sequence with at least 80% sequence identity to SEQ ID NO: 74 or SEQ ID NO: 194; (v) CR5 comprises a nucleic acid sequence with at least 80% sequence identity to SEQ ID NO: 75 or SEQ ID NO: 195; (vi) CR6 comprises a nucleic acid sequence with at least 80% sequence identity to SEQ ID NO: 76 or SEQ ID NO: 196; (vii) CR7 comprises a nucleic acid sequence with at least 80% sequence identity to SEQ ID NO: 77 or SEQ ID NO: 197; (viii) CR8 comprises a nucleic acid sequence with at least 80% sequence identity to SEQ ID NO: 78 or SEQ ID NO: 198; (ix) CR9 comprises a nucleic acid sequence with at least 80% sequence identity to SEQ ID NO: 79 or SEQ ID NO: 199; and (x) CR10 comprises a nucleic acid sequence with at least 80% sequence identity to SEQ ID NO: 80 or SEQ ID NO: 200.

Embodiment 15. An isolated and purified Lactobacillus paracasei, comprising: a 16S rRNA gene comprising a nucleic acid sequence with at least 97% sequence identity to SEQ ID NO: 61 or SEQ ID NO: 181.

Embodiment 16. An isolated and purified Lactobacillus paracasei having deposit accession number 202103012 or 202103018, or a strain having all of the identifying characteristics of Lactobacillus paracasei 202103012 or 202103018, or a mutant thereof.

Embodiment 17. An isolated and purified Bifidobacterium longum, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 82, SEQ ID NO: 202, or SEQ ID NO: 222; (ii) VR2 comprises a nucleic acid sequence with at least 95% sequence identity to SEQ ID NO: 83, SEQ ID NO: 203, or SEQ ID NO: 223; (iii) VR3 comprises a nucleic acid sequence with at least 95% sequence identity to SEQ ID NO: 84, SEQ ID NO: 204, or SEQ ID NO: 224; (iv) VR4 comprises a nucleic acid sequence with at least 95% sequence identity to SEQ ID NO: 85, SEQ ID NO: 205, or SEQ ID NO: 225; (v) VR5 comprises a nucleic acid sequence with at least 95% sequence identity to SEQ ID NO: 86, SEQ ID NO: 206, or SEQ ID NO: 226; (vi) VR6 comprises a nucleic acid sequence with at least 95% sequence identity to SEQ ID NO: 87, SEQ ID NO: 207, or SEQ ID NO: 227; (vii) VR7 comprises a nucleic acid sequence with at least 95% sequence identity to SEQ ID NO: 88, SEQ ID NO: 208, or SEQ ID NO: 228; (viii) VR8 comprises a nucleic acid sequence with at least 95% sequence identity to SEQ ID NO: 89, SEQ ID NO: 209, or SEQ ID NO: 229; and (ix) VR9 comprises a nucleic acid sequence with at least 95% sequence identity to SEQ ID NO: 90, SEQ ID NO: 210, or SEQ ID NO: 230.

Embodiment 18. The Bifidobacterium longum of embodiment 17, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 91, SEQ ID NO: 211, or SEQ ID NO: 231; (ii) CR2 comprises a nucleic acid sequence with at least 80% sequence identity to SEQ ID NO: 92, SEQ ID NO: 212, or SEQ ID NO: 232; (iii) CR3 comprises a nucleic acid sequence with at least 80% sequence identity to SEQ ID NO: 93, SEQ ID NO: 213, or SEQ ID NO: 233; (iv) CR4 comprises a nucleic acid sequence with at least 80% sequence identity to SEQ ID NO: 94, SEQ ID NO: 214, or SEQ ID NO: 234; (v) CR5 comprises a nucleic acid sequence with at least 80% sequence identity to SEQ ID NO: 95, SEQ ID NO: 215, or SEQ ID NO: 235; (vi) CR6 comprises a nucleic acid sequence with at least 80% sequence identity to SEQ ID NO: 96, SEQ ID NO: 216, or SEQ ID NO: 236; (vii) CR7 comprises a nucleic acid sequence with at least 80% sequence identity to SEQ ID NO: 97, SEQ ID NO: 217, or SEQ ID NO: 237; (viii) CR8 comprises a nucleic acid sequence with at least 80% sequence identity to SEQ ID NO: 98, SEQ ID NO: 218, or SEQ ID NO: 238; (ix) CR9 comprises a nucleic acid sequence with at least 80% sequence identity to SEQ ID NO: 99, SEQ ID NO: 219, or SEQ ID NO: 239; and (x) CR10 comprises a nucleic acid sequence with at least 80% sequence identity to SEQ ID NO: 100, SEQ ID NO: 220, or SEQ ID NO: 240.

Embodiment 19. An isolated and purified Bifidobacterium longum, comprising: a 16S rRNA gene comprising a nucleic acid sequence with at least 97% sequence identity to SEQ ID NO: 81, SEQ ID NO: 201, or SEQ ID NO: 221.

Embodiment 20. An isolated and purified Bifidobacterium longum having deposit accession number 202103014, 202103013, or 202103015, or a strain having all of the identifying characteristics of Bifidobacterium longum 202103014, 202103013, or 202103015, or a mutant thereof.

Embodiment 21. An isolated and purified Bifidobacterium animalis lactis, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 102; (ii) VR2 comprises a nucleic acid sequence with at least 95% sequence identity to SEQ ID NO: 103; (iii) VR3 comprises a nucleic acid sequence with at least 95% sequence identity to SEQ ID NO: 104; (iv) VR4 comprises a nucleic acid sequence with at least 95% sequence identity to SEQ ID NO: 105; (v) VR5 comprises a nucleic acid sequence with at least 95% sequence identity to SEQ ID NO: 106; (vi) VR6 comprises a nucleic acid sequence with at least 95% sequence identity to SEQ ID NO: 107; (vii) VR7 comprises a nucleic acid sequence with at least 95% sequence identity to SEQ ID NO: 108; (viii) VR8 comprises a nucleic acid sequence with at least 95% sequence identity to SEQ ID NO: 109; and (ix) VR9 comprises a nucleic acid sequence with at least 95% sequence identity to SEQ ID NO: 110.

Embodiment 22. The Bifidobacterium animalis lactis of embodiment 21, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 111; (ii) CR2 comprises a nucleic acid sequence with at least 80% sequence identity to SEQ ID NO: 112; (iii) CR3 comprises a nucleic acid sequence with at least 80% sequence identity to SEQ ID NO: 113; (iv) CR4 comprises a nucleic acid sequence with at least 80% sequence identity to SEQ ID NO: 114; (v) CR5 comprises a nucleic acid sequence with at least 80% sequence identity to SEQ ID NO: 115; (vi) CR6 comprises a nucleic acid sequence with at least 80% sequence identity to SEQ ID NO: 116; (vii) CR7 comprises a nucleic acid sequence with at least 80% sequence identity to SEQ ID NO: 117; (viii) CR8 comprises a nucleic acid sequence with at least 80% sequence identity to SEQ ID NO: 118; (ix) CR9 comprises a nucleic acid sequence with at least 80% sequence identity to SEQ ID NO: 119; and (x) CR10 comprises a nucleic acid sequence with at least 80% sequence identity to SEQ ID NO: 120.

Embodiment 23. An isolated and purified Bifidobacterium animalis lactis, comprising: a 16S rRNA gene comprising a nucleic acid sequence with at least 97% sequence identity to SEQ ID NO: 101.

Embodiment 24. An isolated and purified Bifidobacterium animalis lactis having deposit accession number 202103016, 202103017, or 202103019, or a strain having all of the identifying characteristics of Bifidobacterium animalis lactis 202103016, 202103017, or 202103019, or a mutant thereof.

Embodiment 25. A composition comprising the Lactobacillus plantarum of any one of embodiments 1-4.

Embodiment 26. A composition comprising the Lactobacillus rhamnosus of any one of embodiments 5-8.

Embodiment 27. A composition comprising the Lactobacillus acidophilus of any one of embodiments 9-12.

Embodiment 28. A composition comprising the Lactobacillus paracasei of any one of embodiments 13-16.

Embodiment 29. A composition comprising the Bifidobacterium longum of any one of embodiments 17-20.

Embodiment 30. A composition comprising the Bifidobacterium animalis lactis of any one of embodiments 21-24.

Embodiment 31. A composition comprising the Lactobacillus sp. of any one of embodiments 1-16.

Embodiment 32. A composition comprising the Bifidobacterium sp. of any one of embodiments 17-24.

Embodiment 33. A composition comprising:
(a) a purified Lactobacillus plantarum comprising a 16S rRNA nucleic acid sequence that is at least 97% identical to SEQ ID NO: 1;
(b) a purified Lactobacillus rhamnosus comprising a 16S rRNA nucleic acid sequence that is at least 97% identical to SEQ ID NO: 21;
(c) a purified Lactobacillus acidophilus comprising a 16S rRNA nucleic acid sequence that is at least 97% identical to SEQ ID NO: 41;
(d) a purified Lactobacillus paracasei comprising a 16S rRNA nucleic acid sequence that is at least 97% identical to SEQ ID NO: 61 or SEQ ID NO: 181;
(e) a purified Bifidobacterium longum comprising a 16S rRNA nucleic acid sequence that is at least 97% identical to SEQ ID NO: 81, SEQ ID NO: 201, or SEQ ID NO: 221;
(f) a purified Bifidobacterium animalis lactis comprising a 16S rRNA nucleic acid sequence that is at least 97% identical to SEQ ID NO: 101;
(g) a purified Veillonella dispar comprising a 16S rRNA nucleic acid sequence that is at least 97% identical to SEQ ID NO: 121;
(h) a purified Veillonella parvula comprising a 16S rRNA nucleic acid sequence that is at least 97% identical to SEQ ID NO: 141; and/or
(i) a purified Veillonella atypica comprising a 16S rRNA nucleic acid sequence that is at least 97% identical to SEQ ID NO: 161.

Embodiment 34. A composition comprising:
(a) a purified Lactobacillus plantarum comprising a 16S rRNA nucleic acid sequence that is at least 97% identical to SEQ ID NO: 1;
(b) a purified Lactobacillus rhamnosus comprising a 16S rRNA nucleic acid sequence that is at least 97% identical to SEQ ID NO: 21; and/or
(c) a purified Lactobacillus acidophilus comprising a 16S rRNA nucleic acid sequence that is at least 97% identical to SEQ ID NO: 41.

Embodiment 35. A composition comprising:
(a) a purified Lactobacillus plantarum comprising a 16S rRNA nucleic acid sequence that is at least 97% identical to SEQ ID NO: 1;
(b) a purified Lactobacillus rhamnosus comprising a 16S rRNA nucleic acid sequence that is at least 97% identical to SEQ ID NO: 21;
(c) a purified Lactobacillus acidophilus comprising a 16S rRNA nucleic acid sequence that is at least 97% identical to SEQ ID NO: 41;
(d) a purified Bifidobacterium longum comprising a 16S rRNA nucleic acid sequence that is at least 97% identical to SEQ ID NO: 81, SEQ ID NO: 201, or SEQ ID NO: 221; and/or
(e) a purified Bifidobacterium animalis lactis comprising a 16S rRNA nucleic acid sequence that is at least 97% identical to SEQ ID NO: 101.

Embodiment 36. A composition comprising:
(a) a purified Lactobacillus plantarum having the deposit accession number 202103009;
(b) a purified Lactobacillus rhamnosus having the deposit accession number 202103010;
(c) a purified Lactobacillus acidophilus having the deposit accession number 202103008;
(d) a purified Lactobacillus paracasei having the deposit accession number 202103012 or 202103018;
(e) a purified Bifidobacterium longum having the deposit accession number 202103013, 202103014, or 202103015;
(f) a purified Bifidobacterium animalis lactis having the deposit accession number 202103016, 202103017, or 202103019;
(g) a purified Veillonella dispar having the deposit accession number PTA-126861;
(h) a purified Veillonella parvula having the deposit accession number PTA-126859; and/or
(i) a purified Veillonella atypica having the deposit accession number PTA-126860.

Embodiment 37. The composition of any one of embodiments 25-36, wherein the composition is administered at a dose that alters the microbiome, enhances exercise endurance, improves athletic performance, increases muscle mass, increases muscular strength, prevents the loss of muscle mass, enhances physical recovery, improves digestion, improves metabolism, increases immunity, reduces inflammation, and/or improves sleep quality in a subject in need thereof.

Embodiment 38. The composition of any one of embodiments 25-36, wherein the composition is a food composition, a beverage composition or a dietary supplement composition.

Embodiment 39. A method of altering the microbiome of a subject comprising administering an effective dose of the composition of any one of embodiments 25-36 to the subject.

Embodiment 40. A method of reducing the level of lactic acid and/or lactates in blood of a subject comprising administering an effective dose of the composition of any one of embodiments 25-36 to the subject.

Embodiment 41. A method of increasing the level of propionic acid and/or propionates in blood of a subject comprising administering an effective dose of the composition of any one of embodiments 25-36 to the subject.

Embodiment 42. A method of increasing the level of acetic acid and/or acetates in blood of a subject comprising administering an effective dose of the composition of any one of embodiments 25-36 to the subject.

Embodiment 43. A method of enhancing exercise endurance of a subject comprising administering an effective dose of the composition of any one of embodiments 25-36 to the subject.

Embodiment 44. A method of improving athletic performance of a subject in need thereof comprising administering an effective dose of the composition of any one of embodiments 25-36 to the subject.

Embodiment 45. A method of enhancing recovery from physical exercise in a subject comprising administering an effective dose of the composition of any one of embodiments 25-36 to the subject.

Embodiment 46. A method of reducing inflammation in a subject in need thereof comprising administering an effective dose of the composition of any one of embodiments 25-36 to the subject.

Embodiment 47. A method of increasing muscle mass and/or muscular strength of a subject in need thereof comprising administering an effective dose of the composition of any one of embodiments 25-36 to the subject.

Embodiment 48. A method of preventing the loss of muscle mass of a subject in need thereof comprising administering an effective dose of the composition of any one of embodiments 25-36 to the subject.

Embodiment 49. A method of improving sleepy quality in a subject in need thereof comprising administering an effective dose of the composition of any one of embodiments 25-36 to the subject.

Embodiment 50. The method of any one of embodiments 39-49, wherein the administration is via oral, enteric, gastrointestinal, or rectal route.

Embodiment 51. The method of any one of embodiments 39-49, wherein the subject is a human subject.

Embodiment 52. The method of any one of embodiments 39-49, wherein the dose is in the range of about 10⁴ CFU to about 10¹⁶ CFU.

Embodiment 53. The method of any one of embodiments 39-49, wherein the dose is in the range of about 10⁹ CFU to about 10¹¹ CFU.

Embodiment 54. The method of any one of embodiments 39-49, wherein the dose is in the range of about 5×10⁹ CFU to about 10¹⁰ CFU.

Embodiment 55. The composition of any one of embodiments 39-49, wherein the dose is about 3.5×10¹⁰ CFU.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. An isolated and purified Lactobacillus plantarum, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 2; (ii) VR2 comprises a nucleic acid sequence with at least 95% sequence identity to SEQ ID NO: 3; (iii) VR3 comprises a nucleic acid sequence with at least 95% sequence identity to SEQ ID NO: 4; (iv) VR4 comprises a nucleic acid sequence with at least 95% sequence identity to SEQ ID NO: 5; (v) VR5 comprises a nucleic acid sequence with at least 95% sequence identity to SEQ ID NO: 6; (vi) VR6 comprises a nucleic acid sequence with at least 95% sequence identity to SEQ ID NO: 7; (vii) VR7 comprises a nucleic acid sequence with at least 95% sequence identity to SEQ ID NO: 8; (viii) VR8 comprises a nucleic acid sequence with at least 95% sequence identity to SEQ ID NO: 9; and (ix) VR9 comprises a nucleic acid sequence with at least 95% sequence identity to SEQ ID NO: 10.

2. The Lactobacillus plantarum of claim 1, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 11; (ii) CR2 comprises a nucleic acid sequence with at least 80% sequence identity to SEQ ID NO: 12; (iii) CR3 comprises a nucleic acid sequence with at least 80% sequence identity to SEQ ID NO: 13; (iv) CR4 comprises a nucleic acid sequence with at least 80% sequence identity to SEQ ID NO:14; (v) CR5 comprises a nucleic acid sequence with at least 80% sequence identity to SEQ ID NO: 15; (vi) CR6 comprises a nucleic acid sequence with at least 80% sequence identity to SEQ ID NO: 16; (vii) CR7 comprises a nucleic acid sequence with at least 80% sequence identity to SEQ ID NO: 17; (viii) CR8 comprises a nucleic acid sequence with at least 80% sequence identity to SEQ ID NO: 18; (ix) CR9 comprises a nucleic acid sequence with at least 80% sequence identity to SEQ ID NO: 19; and (x) CR10 comprises a nucleic acid sequence with at least 80% sequence identity to SEQ ID NO: 20.

3. An isolated and purified Lactobacillus plantarum, comprising: a 16S rRNA gene comprising a nucleic acid sequence with at least 97% sequence identity to SEQ ID NO:

1.

4. An isolated and purified Lactobacillus plantarum having deposit accession number 202103009, or a strain having all of the identifying characteristics of Lactobacillus plantarum 202103009, or a mutant thereof.

5. An isolated and purified Lactobacillus rhamnosus, comprising: a 16S rRNA gene comprising a nucleic acid sequence comprising at least one variable region (VR) selected from the group consisting of VR1, VR2, VR3, VR4, VR5, VR6, VR7, VR8, VR9, and any combination thereof, wherein (i) VR1 comprises a nucleic acid sequence with at least 95% sequence identity to SEQ ID NO: 22; (ii) VR2 comprises a nucleic acid sequence with at least 95% sequence identity to SEQ ID NO: 23; (iii) VR3 comprises a nucleic acid sequence with at least 95% sequence identity to SEQ ID NO: 24; (iv) VR4 comprises a nucleic acid sequence with at least 95% sequence identity to SEQ ID NO: 25; (v) VR5 comprises a nucleic acid sequence with at least 95% sequence identity to SEQ ID NO: 26; (vi) VR6 comprises a nucleic acid sequence with at least 95% sequence identity to SEQ ID NO: 27; (vii) VR7 comprises a nucleic acid sequence with at least 95% sequence identity to SEQ ID NO: 28; (viii) VR8 comprises a nucleic acid sequence with at least 95% sequence identity to SEQ ID NO: 29; and (ix) VR9 comprises a nucleic acid sequence with at least 95% sequence identity to SEQ ID NO: 30.

6. The Lactobacillus rhamnosus of claim 5, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 31; (ii) CR2 comprises a nucleic acid sequence with at least 80% sequence identity to SEQ ID NO: 32; (iii) CR3 comprises a nucleic acid sequence with at least 80% sequence identity to SEQ ID NO: 33; (iv) CR4 comprises a nucleic acid sequence with at least 80% sequence identity to SEQ ID NO: 34; (v) CR5 comprises a nucleic acid sequence with at least 80% sequence identity to SEQ ID NO: 35; (vi) CR6 comprises a nucleic acid sequence with at least 80% sequence identity to SEQ ID NO: 36; (vii) CR7 comprises a nucleic acid sequence with at least 80% sequence identity to SEQ ID NO: 37; (viii) CR8 comprises a nucleic acid sequence with at least 80% sequence identity to SEQ ID NO: 38; (ix) CR9 comprises a nucleic acid sequence with at least 80% sequence identity to SEQ ID NO: 39; and (x) CR10 comprises a nucleic acid sequence with at least 80% sequence identity to SEQ ID NO: 40.

7. An isolated and purified Lactobacillus rhamnosus, comprising: a 16S rRNA gene comprising a nucleic acid sequence with at least 97% sequence identity to SEQ ID NO: 21.

8. An isolated and purified Lactobacillus rhamnosus having deposit accession number 202103010, or a strain having all of the identifying characteristics of Lactobacillus rhamnosus 202103010, or a mutant thereof.

9. An isolated and purified Lactobacillus acidophilus, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 42; (ii) VR2 comprises a nucleic acid sequence with at least 95% sequence identity to SEQ ID NO: 43; (iii) VR3 comprises a nucleic acid sequence with at least 95% sequence identity to SEQ ID NO: 44; (iv) VR4 comprises a nucleic acid sequence with at least 95% sequence identity to SEQ ID NO: 45; (v) VR5 comprises a nucleic acid sequence with at least 95% sequence identity to SEQ ID NO: 46; (vi) VR6 comprises a nucleic acid sequence with at least 95% sequence identity to SEQ ID NO: 47; (vii) VR7 comprises a nucleic acid sequence with at least 95% sequence identity to SEQ ID NO: 48; (viii) VR8 comprises a nucleic acid sequence with at least 95% sequence identity to SEQ ID NO: 49; and (ix) VR9 comprises a nucleic acid sequence with at least 95% sequence identity to SEQ ID NO: 50.

10. The Lactobacillus acidophilus of claim 9, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 51; (ii) CR2 comprises a nucleic acid sequence with at least 80% sequence identity to SEQ ID NO: 52; (iii) CR3 comprises a nucleic acid sequence with at least 80% sequence identity to SEQ ID NO: 53; (iv) CR4 comprises a nucleic acid sequence with at least 80% sequence identity to SEQ ID NO: 54; (v) CR5 comprises a nucleic acid sequence with at least 80% sequence identity to SEQ ID NO: 55; (vi) CR6 comprises a nucleic acid sequence with at least 80% sequence identity to SEQ ID NO: 56; (vii) CR7 comprises a nucleic acid sequence with at least 80% sequence identity to SEQ ID NO: 57; (viii) CR8 comprises a nucleic acid sequence with at least 80% sequence identity to SEQ ID NO: 58; (ix) CR9 comprises a nucleic acid sequence with at least 80% sequence identity to SEQ ID NO: 59; and (x) CR10 comprises a nucleic acid sequence with at least 80% sequence identity to SEQ ID NO: 60.

11. An isolated and purified Lactobacillus acidophilus, comprising: a 16S rRNA gene comprising a nucleic acid sequence with at least 97% sequence identity to SEQ ID NO: 41.

12. An isolated and purified Lactobacillus acidophilus having deposit accession number 202103008, or a strain having all of the identifying characteristics of Lactobacillus acidophilus 202103008, or a mutant thereof.

13. An isolated and purified Lactobacillus paracasei, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 62 or SEQ ID NO: 182; (ii) VR2 comprises a nucleic acid sequence with at least 95% sequence identity to SEQ ID NO: 63 or SEQ ID NO: 183; (iii) VR3 comprises a nucleic acid sequence with at least 95% sequence identity to SEQ ID NO: 64 or SEQ ID NO: 184; (iv) VR4 comprises a nucleic acid sequence with at least 95% sequence identity to SEQ ID NO: 65 or SEQ ID NO: 185; (v) VR5 comprises a nucleic acid sequence with at least 95% sequence identity to SEQ ID NO: 66 or SEQ ID NO: 186; (vi) VR6 comprises a nucleic acid sequence with at least 95% sequence identity to SEQ ID NO: 67 or SEQ ID NO: 187; (vii) VR7 comprises a nucleic acid sequence with at least 95% sequence identity to SEQ ID NO: 68 or SEQ ID NO: 188; (viii) VR8 comprises a nucleic acid sequence with at least 95% sequence identity to SEQ ID NO: 69 or SEQ ID NO: 189; and (ix) VR9 comprises a nucleic acid sequence with at least 95% sequence identity to SEQ ID NO: 70 or SEQ ID NO: 190.

14. The Lactobacillus paracasei of claim 13, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 71 or SEQ ID NO: 191; (ii) CR2 comprises a nucleic acid sequence with at least 80% sequence identity to SEQ ID NO: 72 or SEQ ID NO: 192; (iii) CR3 comprises a nucleic acid sequence with at least 80% sequence identity to SEQ ID NO: 73 or SEQ ID NO: 193; (iv) CR4 comprises a nucleic acid sequence with at least 80% sequence identity to SEQ ID NO: 74 or SEQ ID NO: 194; (v) CR5 comprises a nucleic acid sequence with at least 80% sequence identity to SEQ ID NO: 75 or SEQ ID NO: 195; (vi) CR6 comprises a nucleic acid sequence with at least 80% sequence identity to SEQ ID NO: 76 or SEQ ID NO: 196; (vii) CR7 comprises a nucleic acid sequence with at least 80% sequence identity to SEQ ID NO: 77 or SEQ ID NO: 197; (viii) CR8 comprises a nucleic acid sequence with at least 80% sequence identity to SEQ ID NO: 78 or SEQ ID NO: 198; (ix) CR9 comprises a nucleic acid sequence with at least 80% sequence identity to SEQ ID NO: 79 or SEQ ID NO: 199; and (x) CR10 comprises a nucleic acid sequence with at least 80% sequence identity to SEQ ID NO: 80 or SEQ ID NO: 200.

15. An isolated and purified Lactobacillus paracasei, comprising: a 16S rRNA gene comprising a nucleic acid sequence with at least 97% sequence identity to SEQ ID NO: 61 or SEQ ID NO: 181.

16. An isolated and purified Lactobacillus paracasei having deposit accession number 202103012 or 202103018, or a strain having all of the identifying characteristics of Lactobacillus paracasei 202103012 or 202103018, or a mutant thereof.

17. An isolated and purified Bifidobacterium longum, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 82, SEQ ID NO: 202, or SEQ ID NO: 222; (ii) VR2 comprises a nucleic acid sequence with at least 95% sequence identity to SEQ ID NO: 83, SEQ ID NO: 203, or SEQ ID NO: 223; (iii) VR3 comprises a nucleic acid sequence with at least 95% sequence identity to SEQ ID NO: 84, SEQ ID NO: 204, or SEQ ID NO: 224; (iv) VR4 comprises a nucleic acid sequence with at least 95% sequence identity to SEQ ID NO: 85, SEQ ID NO: 205, or SEQ ID NO: 225; (v) VR5 comprises a nucleic acid sequence with at least 95% sequence identity to SEQ ID NO: 86, SEQ ID NO: 206, or SEQ ID NO: 226; (vi) VR6 comprises a nucleic acid sequence with at least 95% sequence identity to SEQ ID NO: 87, SEQ ID NO: 207, or SEQ ID NO: 227; (vii) VR7 comprises a nucleic acid sequence with at least 95% sequence identity to SEQ ID NO: 88, SEQ ID NO: 208, or SEQ ID NO: 228; (viii) VR8 comprises a nucleic acid sequence with at least 95% sequence identity to SEQ ID NO: 89, SEQ ID NO: 209, or SEQ ID NO: 229; and (ix) VR9 comprises a nucleic acid sequence with at least 95% sequence identity to SEQ ID NO: 90, SEQ ID NO: 210, or SEQ ID NO: 230.

18. The Bifidobacterium longum of claim 17, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 91, SEQ ID NO: 211, or SEQ ID NO: 231; (ii) CR2 comprises a nucleic acid sequence with at least 80% sequence identity to SEQ ID NO: 92, SEQ ID NO: 212, or SEQ ID NO: 232; (iii) CR3 comprises a nucleic acid sequence with at least 80% sequence identity to SEQ ID NO: 93, SEQ ID NO: 213, or SEQ ID NO: 233; (iv) CR4 comprises a nucleic acid sequence with at least 80% sequence identity to SEQ ID NO: 94, SEQ ID NO: 214, or SEQ ID NO: 234; (v) CR5 comprises a nucleic acid sequence with at least 80% sequence identity to SEQ ID NO: 95, SEQ ID NO: 215, or SEQ ID NO: 235; (vi) CR6 comprises a nucleic acid sequence with at least 80% sequence identity to SEQ ID NO: 96, SEQ ID NO: 216, or SEQ ID NO: 236; (vii) CR7 comprises a nucleic acid sequence with at least 80% sequence identity to SEQ ID NO: 97, SEQ ID NO: 217, or SEQ ID NO: 237; (viii) CR8 comprises a nucleic acid sequence with at least 80% sequence identity to SEQ ID NO: 98, SEQ ID NO: 218, or SEQ ID NO: 238; (ix) CR9 comprises a nucleic acid sequence with at least 80% sequence identity to SEQ ID NO: 99, SEQ ID NO: 219, or SEQ ID NO: 239; and (x) CR10 comprises a nucleic acid sequence with at least 80% sequence identity to SEQ ID NO: 100, SEQ ID NO: 220, or SEQ ID NO: 240.

19. An isolated and purified Bifidobacterium longum, comprising: a 16S rRNA gene comprising a nucleic acid sequence with at least 97% sequence identity to SEQ ID NO: 81, SEQ ID NO: 201, or SEQ ID NO: 221.

20. An isolated and purified Bifidobacterium longum having deposit accession number 202103013, 202103014, 202103015, or a strain having all of the identifying characteristics of Bifidobacterium longum 202103013, 202103014, 202103015, or a mutant thereof.

21. An isolated and purified Bifidobacterium animalis lactis, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 95% sequence identity to SEQ ID NO: 102; (ii) VR2 comprises a nucleic acid sequence with at least 95% sequence identity to SEQ ID NO: 103; (iii) VR3 comprises a nucleic acid sequence with at least 95% sequence identity to SEQ ID NO: 104; (iv) VR4 comprises a nucleic acid sequence with at least 95% sequence identity to SEQ ID NO: 105; (v) VR5 comprises a nucleic acid sequence with at least 95% sequence identity to SEQ ID NO: 106; (vi) VR6 comprises a nucleic acid sequence with at least 95% sequence identity to SEQ ID NO: 107; (vii) VR7 comprises a nucleic acid sequence with at least 95% sequence identity to SEQ ID NO: 108; (viii) VR8 comprises a nucleic acid sequence with at least 95% sequence identity to SEQ ID NO: 109; and (ix) VR9 comprises a nucleic acid sequence with at least 95% sequence identity to SEQ ID NO: 110.

22. The Bifidobacterium animalis lactis of claim 21, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 111; (ii) CR2 comprises a nucleic acid sequence with at least 80% sequence identity to SEQ ID NO: 112; (iii) CR3 comprises a nucleic acid sequence with at least 80% sequence identity to SEQ ID NO: 113; (iv) CR4 comprises a nucleic acid sequence with at least 80% sequence identity to SEQ ID NO: 114; (v) CR5 comprises a nucleic acid sequence with at least 80% sequence identity to SEQ ID NO: 115; (vi) CR6 comprises a nucleic acid sequence with at least 80% sequence identity to SEQ ID NO: 116; (vii) CR7 comprises a nucleic acid sequence with at least 80% sequence identity to SEQ ID NO: 117; (viii) CR8 comprises a nucleic acid sequence with at least 80% sequence identity to SEQ ID NO: 118; (ix) CR9 comprises a nucleic acid sequence with at least 80% sequence identity to SEQ ID NO: 119; and (x) CR10 comprises a nucleic acid sequence with at least 80% sequence identity to SEQ ID NO: 120.

23. An isolated and purified Bifidobacterium animalis lactis, comprising: a 16S rRNA gene comprising a nucleic acid sequence with at least 97% sequence identity to SEQ ID NO: 101.

24. An isolated and purified Bifidobacterium animalis lactis having deposit accession number 202103016, 202103017, or 202103019, or a strain having all of the identifying characteristics of Bifidobacterium animalis lactis 202103016, 202103017, or 202103019, or a mutant thereof.

25. A composition comprising the Lactobacillus plantarum of claim 3.

26. A composition comprising the Lactobacillus rhamnosus of claim 7.

27. A composition comprising the Lactobacillus acidophilus of claim 11.

28. A composition comprising the Lactobacillus paracasei of claim 15.

29. A composition comprising the Bifidobacterium longum of claim 19.

30. A composition comprising the Bifidobacterium animalis lactis of claim 23.

31. A composition comprising the Lactobacillus sp. of any one of claims 1-16.

32. A composition comprising the Bifidobacterium sp. of any one of claims 17-24.

33. A composition comprising:
(a) a purified Lactobacillus plantarum comprising a 16S rRNA nucleic acid sequence that is at least 97% identical to SEQ ID NO: 1;
(b) a purified Lactobacillus rhamnosus comprising a 16S rRNA nucleic acid sequence that is at least 97% identical to SEQ ID NO: 21;
(c) a purified Lactobacillus acidophilus comprising a 16S rRNA nucleic acid sequence that is at least 97% identical to SEQ ID NO: 41;
(d) a purified Lactobacillus paracasei comprising a 16S rRNA nucleic acid sequence that is at least 97% identical to SEQ ID NO: 61 or SEQ ID NO: 181;
(e) a purified Bifidobacterium longum comprising a 16S rRNA nucleic acid sequence that is at least 97% identical to SEQ ID NO: 81, SEQ ID NO: 201, or SEQ ID NO: 221;
a purified Bifidobacterium animalis lactis comprising a 16S rRNA nucleic acid sequence that is at least 97% identical to SEQ ID NO: 101;
(g) a purified Veillonella dispar comprising a 16S rRNA nucleic acid sequence that is at least 97% identical to SEQ ID NO: 121;
(h) a purified Veillonella parvula comprising a 16S rRNA nucleic acid sequence that is at least 97% identical to SEQ ID NO: 141; and/or
(i) a purified Veillonella atypica comprising a 16S rRNA nucleic acid sequence that is at least 97% identical to SEQ ID NO: 161.

34. A composition comprising:
(a) a purified Lactobacillus plantarum comprising a 16S rRNA nucleic acid sequence that is at least 97% identical to SEQ ID NO: 1;
(b) a purified Lactobacillus rhamnosus comprising a 16S rRNA nucleic acid sequence that is at least 97% identical to SEQ ID NO: 21; and/or
(c) a purified Lactobacillus acidophilus comprising a 16S rRNA nucleic acid sequence that is at least 97% identical to SEQ ID NO: 41.

35. A composition comprising:
(a) a purified Lactobacillus plantarum comprising a 16S rRNA nucleic acid sequence that is at least 97% identical to SEQ ID NO: 1;
(b) a purified Lactobacillus rhamnosus comprising a 16S rRNA nucleic acid sequence that is at least 97% identical to SEQ ID NO: 21;
(c) a purified Lactobacillus acidophilus comprising a 16S rRNA nucleic acid sequence that is at least 97% identical to SEQ ID NO: 41;
(d) a purified Bifidobacterium longum comprising a 16S rRNA nucleic acid sequence that is at least 97% identical to SEQ ID NO: 81, SEQ ID NO: 201, or SEQ ID NO: 221; and/or
(e) a purified Bifidobacterium animalis lactis comprising a 16S rRNA nucleic acid sequence that is at least 97% identical to SEQ ID NO: 101.

36. A composition comprising:
(a) a purified Lactobacillus plantarum having the deposit accession number 202103009;
(b) a purified Lactobacillus rhamnosus having the deposit accession number 202103010;
(c) a purified Lactobacillus acidophilus having the deposit accession number 202103008;
(d) a purified Lactobacillus paracasei having the deposit accession number 202103012 or 202103018;
(e) a purified Bifidobacterium longum having the deposit accession number 202103013, 202103014, or 202103015;
(f) a purified Bifidobacterium animalis lactis having the deposit accession number 202103016, 202103017, or 202103019;
(g) a purified Veillonella dispar having the deposit accession number PTA-126861;
(h) a purified Veillonella parvula having the deposit accession number PTA-126859; and/or
a purified Veillonella atypica having the deposit accession number PTA-126860.

37. The composition of any one of claims 33-36, wherein the composition is administered at a dose that alters the microbiome, enhances exercise endurance, improves athletic performance, increases muscle mass, increases muscular strength, prevents the loss of muscle mass, enhances physical recovery, improves digestion, improves metabolism, increases immunity, reduces inflammation, and/or improves sleep quality in a subject in need thereof.

38. The composition of claim 33, wherein the composition is a food composition, a beverage composition or a dietary supplement composition.

39. A method of altering the microbiome of a subject comprising administering an effective dose of the composition of claim 33 to the subject.

40. A method of reducing the level of lactic acid and/or lactates in blood of a subject comprising administering an effective dose of the composition of claim 33 to the subject.

41. A method of increasing the level of propionic acid and/or propionates in blood of a subject comprising administering an effective dose of the composition of claim 33 to the subject.

42. A method of increasing the level of acetic acid and/or acetates in blood of a subject comprising administering an effective dose of the composition of claim 33 to the subject.

43. A method of enhancing exercise endurance of a subject comprising administering an effective dose of the composition of claim 33 to the subject.

44. A method of improving athletic performance of a subject in need thereof comprising administering an effective dose of the composition of claim 33 to the subject.

45. A method of enhancing recovery from physical exercise in a subject comprising administering an effective dose of the composition of claim 33 to the subject.

46. A method of reducing inflammation in a subject in need thereof comprising administering an effective dose of the composition of claim 33 to the subject.

47. A method of increasing muscle mass and/or muscular strength of a subject in need thereof comprising administering an effective dose of the composition of claim 33 to the subject.

48. A method of preventing the loss of muscle mass of a subject in need thereof comprising administering an effective dose of the composition of claim 33 to the subject.

49. A method of improving sleepy quality in a subject in need thereof comprising administering an effective dose of the composition of claim 33 to the subject.

50. The method of claim 39, wherein the administration is via oral, enteric, gastrointestinal, or rectal route.

51. The method of claim 39, wherein the subject is a human subject.

52. The method of claim 39, wherein the dose is in the range of about 10⁴ CFU to about 10¹⁶ CFU.

53. The method of claim 39, wherein the dose is in the range of about 10⁹ CFU to about 10¹¹ CFU.

54. The method of claim 39, wherein the dose is in the range of about 5×10⁹ CFU to about 10¹⁰ CFU.

55. The method of claim 39, wherein the dose is about 3.5×10¹⁰ CFU.
